SEQUENCE LISTING (11 sequences)

SEQ ID NO: 1 - protein, 495 aa, Dengue virus
MRCVGIGSRDFVEGLSGATWVDVVLEHGSCVTTMAKDKPTLDIELLKTEVTNPAVLRKLCIEAKISNTTTDSRCPTQGEATLVEEQDANFVCRRTFVDRGWGNGCGLFGKGSLITCAKFKCVTKLEGKIVQYENLKYSVIVTVHTGDQHQVGNESTEHGTTATITPQAPTSEIQLTDYGALTLDCSPRTGLDFNEMVLLTMKEKSWLVHKQWFLDLPLPWTSGASTSQETWNRQDLLVTFKTAHAKKQEVVVLGSQEGAMHTALTGATEIQTSGTTTIFAGHLKCRLKMDKLTLKGMSYVMCTGSFKLEKEVAETQHGTVLVQIKYEGTDAPCKIPFSTQDEKGVTQNGRLITANPIVTDKEKPVNIEAEPPFGESYIVIGAGEKALKLSWFKKGSSIGKMFEATARGARRMAILGDTAWDFGSIGGVFTSVGKLVHQIFGTAYGVLFSGVSWTMKIGIGVLLTWLGLNSRSTSLSMTCIAVGLVTLYLGVMVQA

SEQ ID NO: 2 - protein, 495 aa, Dengue virus
MRCVGIGNRDFVEGLSGATWVDVVLEHGSCVTTMAKNKPTLDIELLKTEVTNPAILRKLCIEAKISNTTTDSRCPTQGEATLVEEQDANFVCRRTFVDRGWGNGCGLFGKGSLLTCAKFKCVTKLEGKIVQYENLKYSVIVTVHTGDQHQVGNETTEHGTIATITPQAPTSEIQLTDYGALTLDCSPRTGLDFNEMVLLTMKEKSWLVHKQWFLDLPLPWTSGASTSQETWNRQDLLVTFKTAHAKKQEVVVLGSQEGAMHTALTGATEIQTSGTTTIFAGHLKCRLKMDKLTLKGTSYVMCTGSFKLEKEVAETQHGTVLVQVKYEGTDAPCKIPFSTQDEKGVTQNGRLITANPIVTDKEKPVNIETEPPFGESYIVVGAGEKALKLSWFKKGSSIGKMFEATARGARRMAILGDTAWDFGSIGGVFTSVGKLVHQVFGTAYGVLFSGVSWTMKIGIGILLTWLGLNSRSTSLSMTCIAVGMVTLYLGVMVQA

SEQ ID NO: 3 - protein, 495 aa, Dengue virus
MRCIGISNRDFVEGVSGGSWVDIVLEHGSCVTTMAKNKPTLDFELIKTEAKQPATLRKYCIEAKLTNTTTESRCPTQGEPSLNEEQDKRFVCKHSMVDRGWGNGCGLFGKGGIVTCAMFTCKKNMEGKIVQPENLEYTIVITPHSGEEHAVGNDTGKHGKEIKITPQSSITEAELTGYGTVTMECSPRTGLDFNEMVLLQMEDKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKIVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMFETTMRGAKRMAILGDTAWDFGSLGGVFTSIGKALHQVFGAIYGAAFSGVSWTMKILIGVIITWIGMNSRSTSLSVSLVLVGVVTLYLGVMVQA

SEQ ID NO: 4 - protein, 493 aa, Dengue virus
MRCVGVGNRDFVEGLSGATWVDVVLEHGGCVTTMAKNKPTLDIELQKTEATQLATLRKLCIEGKITNITTDSRCPTQGEATLPEEQDQNYVCKHTYVDRGWGNGCGLFGKGSLVTCAKFQCLEPIEGKVVQYENLKYTVIITVHTGDQHQVGNDTQGVTVEITPQASTVEAILPEYGTLGLECSPRTGLDFNEMILLTMKNKAWMVHRQWFFDLPLPWTSGATTETPTWNRKELLVTFKNAHAKKQEVVVLGSQEGAMHTALTGATEIQNSGGTSIFAGHLKCRLKMDKLELKGMSYAMCLNTFVLKKEVSETQHGTILIKVEYKGEDAPCKIPFSTEDGQGKAHNGRLITANPVVTKKEEPVNIEAEPPFGESNIVIGIGDKALKINWYKKGSSIGKMFEATARGARRMAILGDTAWDFGSVGGVLNSLGKMVHQIFGSAYTALFSGVSWIMKIGIGVLLTWIGLNSKNTSMSFSCIAIGIITLYLGAVVQA

SEQ ID NO: 5 - protein, 493 aa, Dengue virus
MRCVGVGNRDFVEGLSGATWVDVVLEHGGCVTTMAKNKPTLDIELQKTEATQLATLRKLCIEGKITNITTDSRCPTQGEAVLPEEQDQNYVCKHTYVDRGWGNGCGLFGKGSLVTCAKFQCLEPIEGKVVQYENLKYTVIITVHTGDQHQVGNETQGVTAEITPQASTTEAILPEYGTLGLECSPRTGLDFNEMILLTMKNKAWMVHRQWFFDLPLPWTSGATTETPTWNRKELLVTFKNAHAKKQEVVVLGSQEGAMHTALTGATEIQNSGGTSIFAGHLKCRLKMDKLELKGMSYAMCTNTFVLKKEVSETQHGTILIKVEYKGEDVPCKIPFSTEDGQGKAHNGRLITANPVVTKKEEPVNIEAEPPFGESNIVIGIGDNALKINWYKKGSSIGKMFEATARGARRMAILGDTAWDFGSVGGVLNSLGKMVHQIFGSAYTALFSGVSWVMKIGIGVLLTWIGLNSKNTSMSFSCIAIGIITLYLGAVVQA

SEQ ID NO: 6 - protein, 495 aa, Dengue virus
MRCVGVGNRDFVEGVSGGAWVDLVLEHGGCVTTMAQGKPTLDFELTKTTAKEVALLRTYCIEASISNITTATRCPTQGEPYLKEEQDQQYICRRDVVDRGWGNGCGLFGKGGVVTCAKFSCSGKITGNLVQIENLEYTVVVTVHNGDTHAVGNDTSNHGVTATITPRSPSVEVKLPDYGELTLDCEPRSGIDFNEMILMKMKKKTWLVHKQWFLDLPLPWTAGADTSEVHWNYKERMVTFKVPHAKRQDVTVLGSQEGAMHSALAGATEVDSGDGNHMFAGHLKCKVRMEKLRIKGMSYTMCSGKFSIDKEMAETQHGTTVVKVKYEGAGAPCKVPIEIRDVNKEKVVGRIISSTPFAENTNSVTNIELEPPFGDSYIVIGVGDSALTLHWFRKGSSIGKMFESTYRGAKRMAILGETAWDFGSVGGLFTSLGKAVHQVFGSVYTTMFGGVSWMIRILIGFLVLWIGTNSRNTSMAMTCIAVGGITLFLGFTVQA

SEQ ID NO: 7 - protein, 495 aa, Dengue virus
MRCVGVGNRDFVEGVSGGAWVDLVLEHGGCVTTMAQGKPTLDFELIKTTAKEVALLRTYCIEASISNITTATRCPTQGEPYLKEEQDQQYICRRDMVDRGWGNGCGLFGKGGVVTCAKFSCSGKITGNLVQIENLEYTVVVTVHNGDTHAVGNDTSNHGVTATITPRSPSVEVKLPDYGELTLDCEPRSGIDFNEMILMKMKTKTWLVHKQWFLDLPLPWTAGADTSEVHWNHKERMVTFKVPHAKRQDVTVLGSQEGAMHSALAGATEVDSGDGNHMFAGHLKCKVRMEKLRIKGMSYTMCSGKFSIDKEMAETQHGTTVVKVKYEGTGAPCKVPIEIRDVNKEKVVGRIISSTPFAENTNSVTNIELEPPFGDSYIVIGVGDSALTLHWFRKGSSIGKMFESTYRGAKRMAILGETAWDFGSVGGLLTSLGKAVHQVFGSVYTTMFGGVSWMVRILIGLLVLWIGTNSRNTSMAMSCIAVGGITLFLGFTVHA

SEQ ID NO: 8 - protein, 662 aa, Dengue virus
AFHLTTRGGEPHMIVSKQERGKSLLFKTSAGVNMCTLIAMDLGELCEDTMTYKCPRITEAEPDDVDCWCNATDTWVTYGTCSQTGEHRRDKRSVALAPHVGLGLETRAETWMSSEGAWKQIQKVETWALRHPGFTVIALFLAHAIGTSITQKGIIFILLMLVTPSMAMRCVGIGNRDFVEGLSGATWVDVVLEHGSCVTTMAKNKPTLDIELLKTEVTNPAVLRKLCIEAKISNTTTDSRCPTQGEATLVEEQDANFVCRRTFVDRGWGNGCGLFGKGSLITCAKFKCVTKLEGKIVQYENLKYSVIVTVHTGDQHQVGNETTEHGTTATITPQAPTSEIQLTDYGALTLDCSPRTGLDFNEMVLLTMKEKSWLVHKQWFLDLPLPWTSGASTSQETWNRQDLLVTFKTAHAKKQEVVVLGSQEGAMHTALTGATEIQTSGTTTIFAGHLKCRLKMDKLTLKGMSYVMCTGSFKLEKEVAETQHGTVLVQVKYEGTDAPCKIPFSTQDEKGVTQNGRLITANPIVTDKEKPVNIEAEPPFGESYIVVGAGEKALKLSWFKKGSSIGKMFEATARGARRMAILGDTAWDFGSIGGVFTSVGKLVHQIFGTAYGVLFSGVSWTMKIGIGILLTWLGLNSRSTSLSMTCIAVGMVTLYLGVMVQA

SEQ ID NO: 9 - protein, 662 aa, Dengue virus
MFHLTTRNGEPHMIVSRQEKGKSLLFKTEDGVNMCTLMAMDLGELCEDTITYKCPFLKQNEPEDIDCWCNSTSTWVTYGTCTTTGEHRREKRSVALVPHVGMGLETRTETWMSSEGAWKHAQRIETWILRHPGFTIMAAILAYTIGTTHFQRALIFILLTAVAPSMTMRCIGISNRDFVEGVSGGSWVDIVLEHGSCVTTMAKNKPTLDFELIKTEAKQPATLRKYCIEAKLTNTTTESRCPTQGEPSLNEEQDKRFVCKHSMVDRGWGNGCGLFGKGGIVTCAMFTCKKNMEGKIVQPENLEYTIVITPHSGEEHAVGNDTGKHGKEIKITPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMFETTMRGAKRMAILGDTAWDFGSLGGVFTSIGKALHQVFGAIYGAAFSGVSWTMKILIGVIITWIGMNSRSTSLSVSLVLVGIVTLYLGVMVQA

SEQ ID NO: 10 - protein, 660 aa, Dengue virus
MFHLTSRDGEPRMIVGKNERGKSLLFKTASGINMCTLIAMDLGEMCDDTVTYKCPHITEVEPEDIDCWCNLTSTWVTYGTCNQAGEHRRDKRSVALAPHVGMGLDTRTQTWMSAEGAWRQVEKVETWALRHPGFTILALFLAHYIGTSLTQKVVIFILLMLVTPSMTMRCVGVGNRDFVEGLSGATWVDVVLEHGGCVTTMAKNKPTLDIELQKTEATQLATLRKLCIEGKITNITTDSRCPTQGEAILPEEQDQNYVCKHTYVDRGWGNGCGLFGKGSLVTCAKFQCLEPIEGKVVQHENLKYTVIITVHTGDQHQVGNETQGVTAEITPQASTAEAILPEYGTLGLECSPRTGLDFNEMILLTMKNKAWMVHRQWFFDLPLPWTSGATTETPTWNRKELLVTFKNAHAKKQEVVVLGSQEGAMHTALTGATEIQNSGGTSIFAGHLKCRLKMDKLELKGMSYAMCSNTFVLKKEVSETQHGTILIKVEYKGEDAPCKIPFSTEDGQGKAHNGRLITANPVVTKKEEPVNIEAEPPFGESNIVIGIGDKALKINWYKKGSSIGKMFEATARGARRMAILGDTAWDFGSVGGVLNSLGKMVHQIFGSAYTALFSGVSWIMKIGIGVLLTWIGLNSKNTSMSFSCIAIGIITLYLGAVVQA

SEQ ID NO: 11 - protein, 661 aa, Dengue virus
MFHLSTRDGEPLMIVAKHERGRPLLFKTTEGINKCTLIAMDLGEMCEDTVTYKCPLLVNTEPEDIDCWCNLTSAWVMYGTCTQSGERRREKRSVALTPHSGMGLETRAETWMSSEGAWKHAQRVESWILRNPGFALLAGFMAYMIGQTGIQRTVFFVLMMLVAPSYMRCVGVGNRDFVEGVSGGAWVDLVLEHGGCVTTMAQGKPTLDFELIKTTAKEVALLRTYCIEASISNITTATRCPTQGEPYLKEEQDQQYICRRDVVDRGWGNGCGLFGKGGVVTCAKFSCSGKITGNLVQIENLEYTVVVTVHNGDTHAVGNDTSNHGVTATITPRSPSVEVKLPDYGELTLDCEPRSGIDFNEMILMKMKKKTWLVHKQWFLDLPLPWTAGADTSEVHWNHKERMVTFKVPHAKRQDVTVLGSQEGAMHSALTGATEVDSGDGNHMFAGHLKCKVRMEKLRIKGMSYTMCSGKFSIDKEMAETQHGTTVVKVKYEGTGAPCKVPIEIRDVNKEKVVGRIISSTPFAENTNSVTNIELEPPFGDSYIVIGVGDSALTLHWFRKGSSIGKMFESTYRGAKRMAILGETAWDFGSVGGLFTSLGKAVHQVFGSVYTTMFGGVSWMIRILIGFLVLWIGTNSRNTSMAMTCIAVGGITLFLGFTVQA

(19) United States
(12) Patent Application Publication
(10) Pub. No.: US 2024/0148851 A1
(43) Pub. Date: May 9, 2024

(54) BROADLY REACTIVE IMMUNOGENS OF DENGUE VIRUS, COMPOSITIONS, AND METHODS OF USE THEREOF

(71) Applicant: University of Georgia Research Foundation, Inc., Athens, GA (US)

(72) Inventors: James Daniel ALLEN, Athens, GA (US); Maria Teresa AREVALO, Athens, GA (US); Ted Milburn ROSS, Athens, GA (US); Naoko UNO, Athens, GA (US); Terianne Maiko WONG, Athens, GA (US)

(73) Assignee: University of Georgia Research Foundation, Inc., Athens, GA (US)

(21) Appl. No.: 18/330,194

(22) Filed: Jun. 6, 2023

Related U.S. Application Data
(62) Division of application No. 17/260,032, filed on Jan. 13, 2021, now abandoned.
(60) Provisional application No. 62/697,851, filed on Jul. 13, 2018.

(51) Int. Cl. A61K 39/12; A61P 31/14; A61P 37/04; C07K 14/005

(52) U.S. Cl. CPC A61K 39/12; A61P 31/14; A61P 37/04; C07K 14/005; C12N 2770/24122; C12N 2770/24123; C12N 2770/24134

(57) ABSTRACT

Provided herein are non-naturally occurring, broadly reactive, pan-epitopic antigens derived from Dengue virus that are immunogenic and elicit a broadly reactive immune response, such as a broadly reactive neutralizing antibody response, against Dengue virus subtypes following introduction into a subject. Also provided are non-naturally, broadly reactive occurring immunogens, immunogenic compositions, vaccines, subviral particles (SVPs), and virus-like particles (VLPs) comprising Dengue virus proteins or encoding polynucleotides. Methods of generating an immune response in a subject by administering the immunogens, vaccines, SVPs, VLPs, or immunogenic compositions thereof are provided. In particular, the immunogens comprise a Dengue virus protein, or an antibody binding portion thereof.

### CROSS REFERENCE TO RELATED APPLICATIONS

This application is a division of U.S. application Ser. No. 17/260,032, filed Jan. 13, 2021, which is the U.S. National Stage application, pursuant to 35 U.S.C. § 371, of PCT International Application No. PCT/US2019/041683, filed Jul. 12, 2019, designating the United States and published in English, which claims priority to and benefit of U.S. Provisional Application No. 62/697,851, filed Jul. 13, 2018, the entire contents of each of which are incorporated by reference herein.

### SEQUENCE LISTING

The present application contains a Sequence Listing which has been submitted electronically in XML format following conversion from the originally filed TXT format.

The contents of the electronic XML Sequence Listing, (Date of creation: Jun. 6, 2023; Size: 16,388 bytes; Name: 173093-010603USDIV-Sequence_Listing.xml), and the original TXT format, are herein incorporated by reference in its entirety.

### BACKGROUND

Dengue is an acute febrile disease that is caused by mosquito-borne viruses that has spread rapidly in all regions of the World Health Organization (WHO) in recent years. Dengue virus (DENV) is transmitted by female mosquitoes mainly of the species Aedes aegypti and, to a lesser extent, Aedes albopictus. Dengue is widespread throughout the tropics, with risk of infection varying locally and influenced by rainfall, temperature, and unplanned rapid urbanization.

The classical form of dengue fever (DF) is characterized by high fever, headache, stomachache, rash, myalgia, and arthralgia. Severe dengue, also known as Dengue Hemorrhagic Fever (DHF), and Dengue Shock Syndrome (DSS) are accompanied by thrombocytopenia, vascular leakage, and hypotension. DSS, which can be fatal, is characterized by systemic shock.

Severe dengue or DHF was first recognized in the 1950s during dengue epidemics in the Philippines and Thailand. Before 1970, only nine countries had experienced severe dengue epidemics. Currently, the disease is endemic in more than 100 countries in the WHO regions of Africa, the Americas, the Eastern Mediterranean, Southeast Asia, and the Western Pacific, with the Americas, Southeast Asia and Western Pacific regions most seriously affected. In these regions, dengue has become a leading cause of hospitalization and death among children and adults.

Cases of dengue across the Americas, Southeast Asia and the Western Pacific exceeded 1.2 million in 2008 and over 3.2 million in 2015 (based on official data submitted by Member States). The number of reported cases of dengue has continued to increase. For example, in 2015, 2.35 million cases of dengue were reported in the Americas alone, of which 10,200 cases were diagnosed as severe dengue causing 1,181 deaths.

Not only is the number of cases increasing as the disease spreads to new areas, but explosive outbreaks are occurring. The threat of a possible outbreak of dengue fever now exists in Europe as local transmission was reported for the first time in France and Croatia in 2010 and imported cases were detected in three other European countries. Among travelers returning from low- and middle-income countries, dengue is the second most diagnosed cause of fever after malaria. As estimated by the WHO, 500,000 people with severe dengue require hospitalization each year; with a mortality rate of 2.5% among those who contract the disease.

Dengue virus (DENV) has four distinct, but closely related, serotypes of the virus that cause dengue known as DENV1, DENV2, DENV3 and DENV4. They are genetically and antigenically different. All four of the DENY serotypes are of the Flavivirus genus in the Flaviviridae family. Among the serotypes, there is a 40% difference in amino acids of the Envelope (E) glycoprotein, and a 9% difference within one serotype, resulting in different genotypes. In endemic countries, more than one serotype is in circulation. Recovery from infection by one serotype (or subtype) provides lifelong immunity against that particular serotype (or subtype). However, cross-protective immunity to the other serotypes after recovery is only partial and temporary. Subsequent infections by other serotypes increase the risk of developing severe dengue, while infection with more than one serotype can lead to severe disease and mortality.

Despite intensive research, the underlying mechanisms causing severe dengue are still not well understood, due to the lack of appropriate animal models of infection and disease and to the lack of effective immunogens and vaccines that can increase an individual's defenses against infection by the virus. Developing a vaccine for Dengue virus has been difficult to achieve in view of the phenomenon of antibody dependent enhancement (ADE), in which pre-existing antibodies to one DENV serotype do not neutralize, but instead enhance infection by another DENV serotype. Any potential effective vaccine should protect against all four serotypes of the virus to avoid ADE.

Thus, the development and production of effective immunogens and vaccines directed against all serotypes of Dengue viruses are urgently needed.

### SUMMARY OF THE DISCLOSURE

As described below, non-naturally occurring, broadly reactive antigens and antigen sequences derived from Dengue virus and its subtypes (also called serotypes) are provided. These Dengue virus antigens are potent immunogens that can elicit a broadly reactive immune response against Dengue virus proteins of different serotypes (subtypes), e.g., DENV1-4), and, ultimately, against present and future Dengue virus strains in a subject. As referred to herein, the Dengue virus antigens or antigen sequences that elicit an immune response in a subject are immunogenic antigens or immunogens. These Dengue immunogens are termed broadly reactive and pan-epitopic, because they elicit the production of broadly reactive antibodies that are directed against different subtypes of Dengue viruses having both sequence similarity and variability, and a diversity of epitopes (antigenic determinants) in their antigens and sequences thereof.

In an aspect of the disclosure, the non-naturally occurring Dengue virus antigen amino acid sequences and the antigens comprising the sequences described herein contain broadly reactive epitopes that reflect sequence similarities and variabilities of Dengue virus serotypes 1-4 (DENV1-DENV4) or future serotypes, etc., and/or of past, present and future Dengue antigens. Such antigen sequences and the antigens comprising the sequences are thus called “non-naturally occurring, broadly reactive, pan-epitopic” antigens. The antigens are immunogenic and, when introduced into or administered to a subject, elicit broadly reactive antibodies, such as neutralizing antibodies, against Dengue virus, in particular, Dengue virus antigens(e.g., DENV membrane (M) polypeptide, DENV precursor membrane (prM) polypeptide, DENV envelope (E) polypeptide, DENV prM/E polypeptide, fragments or combinations thereof, etc.) in the subject. In another aspect, the Dengue antigen sequences are polynucleotide sequences, for example, polynucleotide sequences that encode the amino acid sequences of the antigens and immunogens described herein. For ease of reference, a “non-naturally occurring, broadly reactive, pan-epitopic” antigen or immunogen of Dengue virus described herein is interchangeably referred to as a “broadly reactive antigen or immunogen,” or a “pan epitopic antigen or immunogen.”

The broadly reactive Dengue virus antigens described herein are immunogenic as they elicit a broadly reactive immune response in a subject. The immune response is particularly in the form of a neutralizing antibody response, for example, neutralizing antibodies that are specifically directed against the envelope polypeptide antigen of Dengue virus (and its subtypes DENV1, DENV2, DENV3 and DENV4) and that neutralize the activity of the virus. Accordingly, also provided are immunogens and immunogenic compositions that contain the broadly reactive Dengue virus antigens described herein, including vaccines (e.g., DENV polypeptides, DENV polynucleotides, etc.), that induce an immune response directed against Dengue virus, such as, for example, against the envelope (E) protein, membrane (M) protein, precursor membrane (prM) protein, prM/E protein, or fragments or combinations thereof, of Dengue virus, in a subject. For ease of reference, a “non-naturally occurring, broadly reactive, pan-epitopic” Dengue virus immunogen described herein will be referred to as a “broadly reactive immunogen.”

Another aspect of the disclosure is directed to a non-naturally occurring, broadly reactive, pan-epitopic antigen of Dengue virus capable of generating an immune response against one or more Dengue virus subtypes (also known as serotypes) or against present and future Dengue virus strains in a subject. The Dengue virus antigen may be a Dengue virus immunogen, where the immunogen may generate an immune response against one or more Dengue virus subtypes or against present and future Dengue virus strains in a subject. The one or more Dengue virus (DENV or DV) subtypes may be selected from DENV1, DENV2, DENV3, or DENV4, or any future subtype. A further aspect may be directed to an antigen comprising a Dengue virus protein (e.g., DENV membrane (M) protein, DENV precursor membrane (prM) protein, DENV envelope (E) protein, DENV prM/E protein, fragments or combinations thereof, etc.) or an antibody-binding portion thereof.

Also provided in several aspects of the disclosure are methods of using the antigens or immunogens as described herein to induce an immune response against Dengue virus infection, disease, and/or the symptoms thereof in a subject. In a particular aspect, a method of generating an immune response in a subject comprises administering to the subject an effective amount of the Dengue virus antigen, immunogen, VLP, SLP, composition, or the like, of the disclosure. Another aspect may be directed to methods of administering to a subject, the Dengue virus antigen, immunogen, VLP, SLP, composition, or the like, comprising a Dengue virus protein (e.g., DENV M protein, DENV prM protein, DENV E protein, DENV prM/E protein, etc., fragments or combinations thereof, etc.) and its different subtypes (e.g., DENV1, DENV2, DENV3, DENV4, a Dengue virus strain related thereto, etc.) for generating an immune response. Yet in another aspect of the disclosure, methods of generating an immune response in a subject comprising administering to the subject an effective amount of a composition or pharmaceutical composition comprising a Dengue virus antigen, immunogen, VLP, SLP, vaccine, immunogenic composition, etc. are also provided. Additional aspects provide for methods of generating an immune response, where the immune response comprises the production of neutralizing antibodies and/or T-lymphocytes. Other aspects may be directed to such methods further comprising an adjuvant that is concomitantly, essentially simultaneously, or sequentially administered to the subject. Some aspects of the disclosure may provide a Dengue virus antigen or immunogen that is a protein of Dengue virus (e.g., DENV M protein, DENV prM protein, DENV E protein, DENV prM/E protein, etc., or fragments or combinations thereof) and its different subtypes or serotypes (e.g., DENV1, DENV2, DENV3, DENV4, or a Dengue virus strain related thereto, etc.). Methods of using the immunogens to induce an immune response in a subject are also provided.

In an aspect, non-naturally occurring, broadly reactive antigen sequences derived from Dengue virus and its subtypes are provided. The Dengue virus immunogenic antigen may comprise an amino acid sequence that is at least or equal to 80% (e.g., 85%, 90%, 91%, 92%, 93%, 94%, 95%, 96%, 97%, 98%, 99%, etc.) identical to an amino acid sequence of a DENV antigen, including for example, those disclosed herein, a Dengue virus polypeptide (e.g., a Dengue envelope polypeptide, a Dengue virus prM polypeptide, a Dengue virus prM/E polypeptide, etc.) sequence of one or more of the Dengue serotype proteins (e.g., the DENV1, DENV2, DENV3, DENV4, etc.) as set forth in FIG. 1A, FIG. 1B, FIG. 9A, FIG. 9B, SEQ ID NOs: 1-11, or fragments or combinations thereof. In one aspect, the Dengue virus antigen is the Dengue virus envelope polypeptide, Dengue virus prM polypeptide, Dengue virus prM/E polypeptide, or an antibody-binding portion thereof.

In an aspect, the broadly reactive Dengue antigen sequence that is capable of generating an immune response against one or more Dengue virus subtypes and/or against present and future Dengue virus strains or isolates is generated by a method such as described in co-pending provisional patent application No. 62/697,818, filed Jul. 13, 2018, the contents of which are incorporated herein by reference, which involves a consideration of parameters associated with Dengue virus, such as geography and time (e.g., a season, etc.) of infection, for example, amino acid sequences of Dengue virus subtypes and/or strains present in a geographical area or location, such as the Americas or Asia, during a selected period of time (e.g., a linear time range, etc.), in which the Dengue virus was isolated. In brief, the process designs Dengue virus antigen sequences that represent all four Dengue virus serotypes, i.e., DENV1, DENV2, DENV3, and DENV4, and that are broadly reactive immunogens, as they elicit a broad immune response in an immunized host subject. Such a Dengue virus immunogen captures conserved epitopes on the Dengue virus envelope glycoprotein, Dengue virus prM glycoprotein, Dengue virus prM/E glycoprotein, etc. and produces a cross-reactive immune response against one or more DENV serotypes, two or more DENV serotypes, three or more DENV serotypes, or four or more DENV serotypes (or subtypes).

Provided in another aspect is a non-naturally occurring, broadly reactive, pan-epitopic antigen of Dengue virus (of subtype DENV1, DENV2, DENV3, or DENV4) capable of generating an immune response against one or more Dengue virus subtypes and/or against present and future Dengue virus strains or isolates. In an embodiment, the antigen is the Dengue virus envelope (E) protein. In an embodiment, the Dengue virus antigen comprises an amino acid sequence that is at least 95% identical or at least 98% identical to an amino acid sequence of a Dengue virus antigen as set forth in FIG. 1A, FIG. 1B, FIG. 9A, FIG. 9B, or SEQ ID NOs:1-11. In a particular embodiment, the Dengue virus antigen comprises an amino acid sequence, or fragment or combinations thereof, as set forth in FIG. 1A, FIG. 1B, FIG. 9A, FIG. 9B, or SEQ ID NOs:1-11. In a particular embodiment, the Dengue virus antigen is the Dengue virus envelope polypeptide or an antibody-binding portion thereof.

Another aspect of the disclosure may be directed to a virus-like particle (VLP) comprising the Dengue virus antigen of the disclosure. Other aspects may provide a VLP comprising a polynucleotide encoding the Dengue virus antigen disclosed herein. Provided in another aspect is a virus-like particle (VLP) comprising the Dengue virus antigen according to the foregoing aspect. In one aspect, the VLP comprises a polynucleotide encoding the Dengue virus antigen. In another aspect, the VLP Dengue virus antigen is a Dengue virus polypeptide (e.g., DENV membrane (M) polypeptide, DENV precursor membrane (prM) polypeptide, DENV envelope (E) polypeptide, DENV prM/E polypeptide, fragments, or combinations thereof, etc.), or an antibody-binding portion thereof.

A further aspect of the disclosure provides for a subviral particle (SVP) comprising the Dengue virus antigen. In one aspect a SVP comprising a polynucleotide encoding the Dengue virus antigen of the disclosure is provided. Another aspect is directed to a subviral particle (SVP) comprising the Dengue virus antigen according to the foregoing aspect. Subviral particles are produced during infection of cells by the virus. SVPs can be isolated and purified from cells infected with (or transfected with) polynucleotide constructs encoding the Dengue virus antigens described herein. In one aspect, the SVP comprises a polynucleotide encoding the Dengue virus antigen. In yet another aspect, the SVP Dengue virus antigen is a Dengue virus polypeptide (e.g., DENV M polypeptide, DENV prM polypeptide, DENV E polypeptide, DENV prM/E polypeptide, etc., or fragments or combinations thereof), or an antibody-binding portion thereof.

In another aspect, subviral particles (SVPs) and virus-like particles (VLPs) comprising the broadly reactive Dengue immunogen or a sequence thereof are provided. One aspect provides DENV SVPs and VLPs comprising the Dengue virus immunogen or sequence thereof, where the sequence comprises an DENV amino acid sequence.

Another aspect provides for a polynucleotide sequence encoding the Dengue virus antigen of the disclosure is provided. The DENV antigen (e.g., immunogen, SVP, VLP, immunogenic composition, etc.) comprises an amino acid sequence as set forth in FIG. 1A, FIG. 1B, FIG. 9A, FIG. 9B, or SEQ ID NOs:1-11.

One aspect of the disclosure provides for a composition or pharmaceutically acceptable composition comprising the polynucleotide encoding the Dengue virus antigen (e.g., immunogen, SVP, VLP, immunogenic composition, etc.) comprising a polynucleotide sequence encoding an amino acid sequence or an amino acid sequence as set forth in FIG. 1A, FIG. 1B, FIG. 9A, FIG. 9B, or SEQ ID NOs:1-11, and a pharmaceutically acceptable carrier, diluent, or excipient.

Yet a further aspect is directed to a non-naturally occurring, pan-epitopic immunogen capable of generating an immune response against one or more Dengue virus subtypes (e.g., DENV1, DENV2, DENV3, DENV4, etc.) or against present and future Dengue virus strains in a subject. The immunogen may comprise a Dengue virus protein (e.g., DENV membrane (M) protein, DENV precursor membrane (prM) protein, DENV envelope (E) protein, DENV prM/E protein, fragments or combinations thereof, etc.). One aspect may be directed to a Dengue virus immunogen comprises a Dengue virus envelope protein or antibody-binding portion thereof. Another aspect provides a Dengue virus immunogen comprising a Dengue virus prM/E protein or antibody-binding portion thereof. Dengue virus immunogens in a further aspect of the disclosure generate an immune response against one or more Dengue virus subtypes or against present and future Dengue virus strains in a subject.

In another aspect, the Dengue virus antigen, immunogen, VLP, or SVP elicits the production of neutralizing antibodies. In one aspect, the Dengue virus antigen, immunogen, VLP, or SVP elicits the production of T-lymphocytes.

Provided in another aspect is a pharmaceutically acceptable composition comprising the Dengue virus antigen, immunogen, VLP, or SVP of any of the foregoing aspects and a pharmaceutically acceptable carrier, diluent, or excipient. In one aspect, the pharmaceutically acceptable composition further comprises an adjuvant.

Provided in another aspect is a vaccine or an immunogenic composition comprising the broadly reactive Dengue virus antigen, immunogen, VLP, SVP, or combinations thereof, of any of the foregoing aspects, or as disclosed herein, and a carrier. The composition may be a pharmaceutical composition, a pharmaceutically acceptable composition, an immunogenic composition, or the like, where the composition comprises the Dengue virus antigen, immunogen, VLP, or SVP of the disclosure. The composition, pharmaceutically-acceptable composition, pharmaceutical composition, or immunogenic composition comprising the Dengue virus antigen, immunogen, VLP, SVP, or combinations thereof, of the disclosure may additionally comprise a pharmaceutically acceptable carrier (e.g., diluent, excipient, etc.). Another aspect may be directed to the composition (e.g., pharmaceutically acceptable composition, pharmaceutical composition, immunogenic composition, etc.) comprising the Dengue virus antigen, immunogen, VLP, SVP, or combinations thereof, of the disclosure, further comprising an adjuvant.

Provided in another aspect is a method of generating an immune response in a subject, in which the method comprises administering to the subject an effective amount of the antigen or immunogen, composition or pharmaceutically acceptable composition, vaccine, VLP, or SVP of any of the foregoing aspects. In one aspect, the immune response elicited comprises the production of neutralizing antibodies. In an embodiment, the immune response elicited comprises the production of neutralizing antibodies and/or T-lymphocytes. As referred to herein, the Dengue virus antigens or antigen sequences that elicit an immune response in a subject are immunogenic antigens or immunogens. These Dengue immunogens are termed broadly reactive and pan-epitopic because they elicit the production of broadly reactive antibodies that are directed against different subtypes of Dengue viruses having both sequence similarity and variability, and a diversity of epitopes (antigenic determinants) in their antigens and sequences thereof.

In an aspect, the broadly reactive Dengue immunogen is isolated and/or purified. One aspect provides the broadly reactive Dengue immunogen that is formulated for administration to a subject in need thereof. In another aspect, the broadly reactive Dengue immunogen or a composition containing the immunogen is administered to a subject in need thereof in an effective amount to elicit an immune response in the subject. embodiment further aspect of the disclosure may be directed to the immune response that elicits neutralizing antibodies. and/or T-lymphocytes. In yet another aspect of the disclosure, the immune response is prophylactic or therapeutic.

Another aspect directed to the broadly reactive Dengue virus antigens described herein provide for immunogenic Dengue virus antigens as they elicit a broadly reactive immune response in a subject. The immune response is particularly in the form of a neutralizing antibody response, for example, neutralizing antibodies that are specifically directed against the Dengue virus polypeptide antigen (e.g., DENV membrane (M) polypeptide antigen, DENV precursor membrane (prM) polypeptide antigen, DENV envelope (E) polypeptide antigen, DENV prM/E polypeptide antigen, fragments or combinations thereof, etc.) and its subtypes (e.g., DENV1, DENV2, DENV3, DENV4, etc.) and that neutralize the activity of the virus.

In another aspect, an immunogenic composition or vaccine comprising the broadly reactive Dengue immunogen as disclosed herein is provided. Another aspect of the disclosure may provide for a composition comprising the Dengue virus antigen, immunogen, VLP, SVP, or combinations thereof, as disclosed herein, and a carrier. The composition may be a pharmaceutical composition, a pharmaceutically acceptable composition, an immunogenic composition, or the like, where the composition comprises the Dengue virus antigen, immunogen, VLP, or SVP of the disclosure. The composition, pharmaceutically-acceptable composition, pharmaceutical composition, or immunogenic composition comprising the Dengue virus antigen, immunogen, VLP, or SVP of the disclosure may additionally comprise a pharmaceutically acceptable carrier (e.g., diluent, excipient, etc.). Another aspect may be directed to the composition (e.g., pharmaceutically acceptable composition, pharmaceutical composition, immunogenic composition, etc.) comprising the Dengue virus antigen, immunogen, VLP, or SVP of the disclosure, further comprising an adjuvant.

In another aspect, subviral particles (SVPs) and virus-like particles (VLPs) comprising the broadly reactive Dengue virus immunogen or a sequence thereof are provided. In one aspect, the sequence is a Dengue virus amino acid sequence. In yet another aspect, the sequence is a Dengue virus polynucleotide sequence which encodes the Dengue virus amino acid sequence.

In another aspect, a method of generating an immune response in a subject is provided, in which the method comprises administering to the subject an effective amount of the Dengue virus broadly reactive immunogen, vaccine, VLP, SVP, or composition of any of the above aspects. In a further aspect of the method, an adjuvant is concomitantly, simultaneously, or subsequently administered to the subject.

### BRIEF DESCRIPTION OF THE DRAWINGS

FIG. 1A and FIG. 1B show the amino acid sequences of seven representative polypeptides (envelope (E) proteins) of Dengue virus (DENV) subtypes, (also called serotypes), viz, DENV1, DENV2, DENV3 and DENV4 herein, which are broadly reactive immunogens that elicit an immune response against Dengue virus protein. The proteins of the DENV1, DENV2 and DENV4 type Dengue viruses are 495 amino acids in length; the proteins of the DENV3 type Dengue virus are 493 amino acids in length. Nucleic acid sequences encoding these polypeptides can be used to generate virus-like particles (VLPs) containing the Dengue protein antigens, which are used as immunogens/vaccines to generate neutralizing antibodies in immunized subjects.

FIG. 2A and FIG. 2B provide drawings of the Dengue virus (DENV) virion molecule and the life cycle of the Dengue virus, including cell infection. FIG. 2A illustrates the enveloped DENV and its structural proteins, capsid (C), membrane (M) and envelope (E) that comprise the outer coat of the virus. The seven nonstructural (NS) proteins are responsible for viral replication and host immune evasion. FIG. 2B depicts the DENV life cycle from receptor binding, intracellular production of virus protein, assembly, and maturation, and exocytosis of mature virions.

FIG. 3 illustrates a protocol used to immunize animals with a broadly reactive Dengue virus immunogen as described herein. In the protocol, female C57BL/6 mice (n=5), aged 6-8 weeks, were immunized three times with purified (i) SVPs containing wild type (WT) Dengue virus antigen sequences (envelope protein sequence); (ii) SVPs containing broadly reactive antigen sequences (envelope protein sequence) of Dengue virus as described herein; or (iii) with PBS (control). Serum samples were collected and assessed in vitro for seroconversion and the ability to neutralize a panel of dengue viruses (3 strains from each serotype), as described in Example 1 herein.

FIG. 4 shows graphs demonstrating the results of enzyme linked immunosorbent assays (ELISA) to detect serum antibodies directed against DENV SVPs of all serotypes (DV1-4). For the ELISA assay, the wells of plates were coated with SVP of wild-type Dengue virus serotypes 1-4 (WT DV1-4 SVP). Absorbance at 405 nm (y-axis) versus the DENV SVP immunogens administered to mice prior to collecting sera (x-axis) is shown. Seroconversion (the elicitation of antibodies directed against the SVPs) was observed in sera obtained from animals immunized with different subtypes of WT DENV SVPs and in sera obtained from animals immunized with a broadly reactive DENV immunogen as described herein versus sera from control animals receiving PBS. “BR Immun” denotes SVPs comprising a broadly reactive DENV immunogenic sequence as described herein and used as an immunogen.

FIG. 5 shows graphs of SVP vaccine concentration of the four Dengue virus serotypes (DV1-4). SVPs expressing either wild-type or broadly reactive, pan-epitopic DENV E proteins were purified from cell culture supernant following transfection with plasmid DNA containing prM-E gene sequences. Each aliquot of purified DENV SVP vaccine was assessed for protein concentration using a spectrophotometer and the absorbance at 405 nm was determined.

FIG. 6 shows a schematic depiction of a focus (plaque) reduction neutralization assay (FRNT), e.g., Examples 1 and 2, in which serial dilutions (starting with a 1:40 dilution) of mouse sera obtained from animals that had been administered SVPs containing DENV antigen sequences, including a broadly reactive DENV immunogen sequence as described herein, were mixed with Dengue virus (or SVPs), and the mixture was incubated for 60 minutes. The mixtures containing complexes of serum antibodies and virus were then added to Vero cells previously plated on tissue culture (TC) plates or in wells of TC plates, and the TC plates or wells were incubated for 90 minutes. Thereafter, the TC plates or wells were washed and incubated in 1% methylcellulose for 2-3 days. Foci (plaques) were observed on the plates following the incubation and quantified.

FIG. 7 presents graphs showing the results (FRNT₅₀) of focus reduction neutralization assays (FRNT). Sera from mice immunized with SVPs containing wild-type sequences from all four DENV serotypes (WT DV1-4 SVP) were pooled and tested for the ability to neutralize DENV in in vitro FRNT assays. The FRNT assays were performed against a panel of 12 different DENV strains (12-panel strains shown on the x-axis). Focal Reduction Neutralization Titer 50 (FRNT₅₀) is shown on the y-axis. The dotted line on the graphs indicates a 1:40 dilution of sera (titer). In the graphs, DENV1 serotype strains are represented by DV1; DENV2 serotype strains are represented by DV2; DENV3 serotype strains are represented by DV3; and DENV4 serotype strains are represented by DV4.

FIGS. 8A-8C present graphs showing the results of focus reduction neutralization assays (FRNT) using sera from mice immunized with different DENV SVPs. Sera from mice immunized with SVPs containing DENV antigen sequences were assayed against the 12-strain panel of Dengue virus strains as described for FIG. 7. Assayed by FRNT were sera from mice immunized with a tetravalent SVP containing wild-type Dengue virus antigen sequence (WT tet SVP), (FIG. 8A) and sera from mice immunized with SVP containing a broadly reactive DENV immunogen sequence as described herein (Broadly Reactive DENV Immunogen SVP), (FIG. 8B). FIG. 8C shows the results of the FRNT assay using sera from control animals that had received PBS. Sera from animals immunized with Broadly Reactive DENV Immunogen SVPs showed neutralization of all four serotypes of DENV.

FIG. 9A and FIG. 9B show the amino acid sequences of four representative polypeptides comprising the precursor membrane (prM)/Envelope (E) proteins of Dengue virus (DENV) subtypes, (also called serotypes), viz, DENV1, DENV2, DENV3 and DENV4 herein, which are broadly reactive immunogens that elicit an immune response against Dengue virus protein. The prM/E proteins of the DENV1 (COBRA DV1; SEQ ID NO:8), DENV2 (COBRA DV2; SEQ ID NO:9), DENV3 (COBRA DV3; SEQ ID NO:10), and DENV4 (COBRA DV4; SEQ ID NO:11) type Dengue viruses are 662 amino acids in length (i.e., 167 amino acids (prM) and 495 amino acids (E) in length); the prM/E protein of the DENV3 (COBRA DV3; SEQ ID NO:3) type Dengue virus is 661 amino acids in length (i.e., 166 amino acids (prM) and 493 amino acids in length), where the prM portion at the N-terminal end is underlined.

FIGS. 10A-10E show a COBRA schematic of the study and constructs. FIG. 10A demonstrates Dengue virus (DENV) E nucleotide sequences isolated from human infections from 1941 to 2006 that were translated into protein sequences (100 from each serotype) and grouped by phylogenetic clustering and geographic location per serotype. Multiple rounds of sequence alignments were performed to retain common epitopes within the sequence. The bar graphs of FIG. 10B and FIG. 10D show the results of an ELISA for detecting surface expression of DENV E using pan-Dengue monoclonal antibody (mAb) on wild-type (WT) Dengue Virus (DV) (FIG. 10B) and COBRA (FIG. 10D) SVPs. FIG. 10D shows the results of an ELISA to detect DENV E expression using pan-Dengue monoclonal antibody (mAb) using plates that were coated with COBRA E SVPs (1 μg/mL) or commercial Dengue antigens (1 μg/mL). Absorbance was measured at 405 nm. Western blot Detection of E glycoprotein using a Pan-flavivirus monoclonal antibody against WT and COBRA E (FIG. 10C) and COBRA1-COBRA 4 (FIG. 10E) SVPs was demonstrated by agarose gel electrophoresis. FIG. 10C: Lane 1: WT DV1; Lane 2: WT DV2; Lane 3: WT DV3; Lane 4: WT DV4; Lane 5: COBRA E; M: Molecular Weight Marker. FIG. 10E: Lane 1: COBRA 1; Lane 2: COBRA 2; Lane 3: COBRA 3; Lane 4: COBRA 4; M: Molecular Weight Marker.

FIGS. 11A-11D demonstrate a seroconversion of mice given WT and COBRA SVP vaccinations. Each graph represents antibody binding to DV SVP (FIG. 11A) or soluble E (FIG. 11B) representing one of the four dengue virus serotypes. FIG. 11C and FIG. 11D show graphs that represent antibody binding to SVP or soluble commercial DENV E representing one of the four dengue serotypes (DV1-DV4). SVP of WT DV1-4 made in-lab produced broad cross reactivity in all groups. Commercial E DV1-DV4 had narrow, homologous reactivity, with DV1 and DV2 coating producing a more robust response than DV3 or DV4. Total Immunoglobulin G (IgG) binding of sera from individual mice given vaccinations were measured by their optical density (OD) values. Absorbance was measured at 405 nm.

FIGS. 12A-12F illustrate the neutralization ability of WT and COBRA SVP vaccine groups. Focus reduction neutralization test (FRNT) to determine neutralization titer against a panel of prototype, American, and Asian strains from each serotype. FRNT50 indicates the titer at which there is 50% reduction of the virus compared to the no sera control and FRNT80 indicates the titer at which there is 80% reduction of the virus compared to no sera control. FRNT50 (FIG. 12A) and FRNT80 (FIG. 12B) of monovalent WT SVP vaccination groups. FRNT50 (FIG. 12C) and FRNT80 (FIG. 12D) of monovalent COBRA SVP vaccination groups. FRNT50 (FIG. 12E) and FRNT80 (FIG. 12F) of tetravalent WT and COBRA vaccination groups and PBS control group.

FIG. 13 shows longevity of COBRA SVP groups. FRNT50 was performed on top COBRA candidates COBRA 1, COBRA 2, and COBRA E SVP vaccination groups, along with the tetravalent COBRA (COBRA tet) SVP vaccination group, to determine longevity of breadth. Sera was collected 5 months after final vaccination and tested against the same panel of Dengue Virus (DV) DENV1-4. The virus strains are as follows: 1) DV1 prototype; 2) DV1 Modern Amer.; 3) DV1 Modern Asian; 4) DV2 prototype; 5) DV2 Modern Amer.; 6) DV2 Modern Asian; 7) DV3 prototype; 8) DV3 Modern Amer.; 9) DV3 Modern Asian; 10) DV4 prototype; 11) DV4 Modern Amer.; 12) DV4 Modern Asian. A statistical significance was determined by Student's t-test between FRNT done 4 weeks and 20 weeks post last vaccination as indicated by asterisks. ** for P≤0.01, **** for P≤0.0001.

FIG. 14 illustrates IgG subclasses elicited by WT and COBRA SVP vaccines. Each table shows IgG subclass binding of homologous monovalent WT DV SVP, WT DV tetravalent SVP, COBRA1-4, COBRA E SVP, and COBRA tetravalent SVP to commercial E antigen representing DENV1, DENV2, DENV3, and DENV4. Asterisks represent statistical significance measured against PBS control group, measured with unpaired Student's t-test with Welch's correction, with the color intensity increasing with the significance. Not significant (ns) for P>0.05, * for P≤0.05, ** for P≤0.01, *** for P≤0.001, **** for P≤0.0001.

### DETAILED DESCRIPTION OF THE DISCLOSURE

Dengue viruses (DENV) are a significant cause of morbidity and mortality in humans, particularly in tropical, subtropical, wetlands, or other mosquito-ridden regions, where mosquitoes transmit the virus. In order to overcome the antibody dependent enhancement (ADE) phenomenon, in which pre-existing antibodies to one DENV serotype do not neutralize, but instead enhance a heterotypic infection by another DENV serotype, a DENV vaccine that is broadly reactive to Dengue virus is desirable. Embodiments of the disclosure may be directed to antigens, immunogens, subviral particles (SVPs), virus-like particles (VLPs), vaccines or compositions comprising, or polynucleotides encoding, the antigen, immunogen, SVP, or VLP that generate an immune response against one or more Dengue virus serotypes, and methods of use thereof. In one embodiment, a DENV subviral particle (SVP) vaccine targeting the Dengue virus envelope (E) glycoprotein was designed using computationally-optimized broadly reactive antigen (COBRA) methodology (See, e.g., Crevar C J, et al. Hum Vaccin Immunother 11:572-583, 2015; Giles B M, et al. Clin Vaccine Immunol. 19:128-139, 2012; Giles B M, et al. J Infect Dis. 205:1562-1570, 2012; Giles B M and Ross T M. 2011. Vaccine 29:3043-3054, 2016; Carter D M, et al. J. Virol. 90(9):4720-4734, 2016).

### Definitions

Unless defined otherwise, all technical and scientific terms used herein have the meaning commonly understood by a person skilled in the art to which this invention pertains or relates. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); Benjamin Lewin, Genes V, published by Oxford University Press, 1994 (ISBN 0-19-854287-9); Kendrew et al. (eds.); The Encyclopedia of Molecular Biology, published by Blackwell Science Ltd., 1994 (ISBN 0-632-02182-9); Molecular Biology and Biotechnology: a Comprehensive Desk Reference, Robert A. Meyers (ed.), published by VCH Publishers, Inc., 1995 (ISBN 1-56081-569-8); and Hale & Marham, The Harper Collins Dictionary of Biology (1991). As used herein, the following terms have the meanings ascribed to them below, unless specified otherwise.

As used herein, “a” or “an” shall mean one or more. As used herein when used in conjunction with the word “comprising,” the words “a” or “an” mean one or more than one. As used herein “another” means at least a second or more.

By “adjuvant” is meant a substance or vehicle that non-specifically enhances the immune response to an antigen. Adjuvants may include a suspension of minerals (e.g., alum, aluminum hydroxide, phosphate, etc.) on which antigen is adsorbed; or water-in-oil emulsion in which antigen solution is emulsified in mineral oil (e.g., Freund“s incomplete adjuvant, etc.), sometimes with the inclusion of killed mycobacteria (e.g., Freund” s complete adjuvant, etc.) to further enhance antigenicity. Immunostimulatory oligonucleotides (such as those including, for example, a CpG motif, etc.) can also be used as adjuvants (see, e.g., U.S. Pat. Nos. 6,194,388; 6,207,646; 6,214,806; 6,218,371; 6,239,116; 6,339,068; 6,406,705; 6,429,199, etc.). Adjuvants also include biological molecules, such as costimulatory molecules. Exemplary biological adjuvants include, without limitation, interleukin-1 (IL-2), the protein memory T-cell attractant “Regulated on Activation, Normal T Expressed and Secreted” (RANTES), granulocyte-macrophage-colony stimulating factor (GM-CSF), tumor necrosis factor-alpha (TNF-α), interferon-gamma (IFN-γ), granulocyte-colony stimulation factor (G-CSF), lymphocyte function-associated antigen 3 (LFA-3, also called CD58), cluster of differentiation antigen 72 (CD72), (a negative regulator of B cell responsiveness), peripheral membrane protein, B7-1 (B7-1, also called CD80), peripheral membrane protein, B7-2 (B7-2, also called CD86), the TNF ligand superfamily member 4 ligand (OX40L), or the type 2 transmembrane glycoprotein receptor belonging to the TNF superfamily (4-1BBL), or the like.

By “administer” is meant giving, supplying, or dispensing a composition, agent, therapeutic, or the like, or combinations thereof, to a subject, or applying or bringing the composition, agent, therapeutic, or the like, or combinations thereof, into contact with a subject. Administering or administration may be accomplished by any of a number of routes, such as, for example, without limitation, topical, oral, subcutaneous, intramuscular, intraperitoneal, intravenous (IV), (injection), intrathecal, intramuscular, dermal, intradermal, intracranial, inhalation, rectal, intravaginal, intraocular, and/or the like.

By “agent” is meant any substance (e.g., small molecule chemical compound, an antibody, a nucleic acid molecule, a peptide, a polypeptide, fragments thereof, etc.).

By “alteration” is meant a change (e.g., increase, decrease, etc.) in the expression levels or activity of a gene or polypeptide as detected by standard art known methods such as those described herein. As used herein, an alteration includes a 5% or greater percent change in expression levels (e.g., a 10%, 15%, 20%, a 25%, 30%, 35%, 40%, 45%, 50%, etc.). “

By “ameliorate” is meant to improve a condition, which, for example, may occur by decreasing (e.g., reducing, diminishing, suppressing, attenuating, arresting, or stabilizing, etc.) the development or progression of a disease or pathological condition.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to the naturally-occurring polypeptide. Such biochemical modifications could, for example, without limitation, increase the analog” s protease resistance, membrane permeability, or half-life, without altering, for example, ligand binding. An analog may include an unnatural amino acid.

By “antibody” is meant an immunoglobulin (Ig) molecule produced by B lymphoid cells and having a specific amino acid sequence. Antibodies are evoked or elicited in subjects (e.g., humans, animals, mammals, etc.) following exposure to a specific antigen (e.g., immunogen, etc.). A subject capable of generating antibodies or immunoglobulin (i.e., an immune response) directed against a specific antigen or immunogen is said to be immunocompetent. Antibodies are characterized by reacting specifically with (e.g., binding to, etc.) an antigen or immunogen in some demonstrable way, antibody and antigen or immunogen each being defined in terms of the other.

“Eliciting an antibody response” refers to the ability of an antigen, immunogen or other molecule to induce the production of antibodies. Antibodies are of different classes (e.g., IgM, IgG, IgA, IgE, IgD, etc.) and subtypes or subclasses (e.g., IgG1, IgG2, IgG2a, IgG2b, IgG3, IgG4, etc.). An antibody/immunoglobulin response elicited in a subject can neutralize a pathogenic (e.g., infectious, disease-causing, etc.) agent by binding to epitopes (e.g., antigenic determinants, etc.) on the agent and blocking or inhibiting the activity of the agent, and/or by forming a binding complex with the agent that is cleared from the system of the subject (e.g., via the liver, etc.).

As used herein, ““broadly reactive”” means that an immune response is elicited against a viral protein (e.g., a virus antigen, virus antigen sequence, virus protein, virus protein sequence, etc.) in a subject that is sufficient to inhibit (e.g., block, impede, neutralize, prevent, etc.) infection of a broad range of related viruses, including for example, without limitation, Dengue viruses (including e.g., Dengue viruses of subtypes DENV1, DENV2, DENV3, DENV4, other Dengue viruses, subtypes, etc.).

By “antigen” is meant a substance (e.g., compound, composition, etc.) that can stimulate the production of antibodies, an immune response, or a T-cell response in a subject, including compositions that are injected or absorbed into animal subject. An antigen may react with the products of specific humoral or cellular immunity, including, for example, those induced by heterologous immunogens. In some embodiments of the disclosure, the antigen is a Dengue viral protein (e.g., DENV 1-DENV4, DENV M protein, DENV prM protein, DENV E protein, DENV prM/E protein, etc., or fragments or combinations thereof, etc. Other embodiments provide an antigen that elicits or stimulates an immune response in a subject is termed an “immunogen.” In yet another embodiment, the immunogen is a Dengue virus protein or fragment thereof sufficient to elicit or stimulate an immune response in a subject.

The term “antigenic drift” refers to a mechanism for variation in organisms or microorganisms such as viruses that involves the accumulation of mutations within the genes that code for antibody-binding sites (also called antigenic determinants or epitopes). This process results in a new strain of virus or virus particles that is not inhibited or blocked as effectively by antibodies that were originally generated against the antigens of virus strains prior to mutation, thus allowing the virus to spread more easily throughout a partially immune population. By way of example, antigenic drift may occur in Dengue virus subtypes, e.g., DENV1, DENV2, DENV3, DENV4, etc.

In the context of a live virus, the term “attenuated” reflects a virus that is attenuated if its ability to infect a cell or subject and/or its ability to produce disease is reduced (for example, diminished, abrogated, eliminated, etc.) compared to the ability of a wild-type virus to produce disease in the cell or subject. Typically, an attenuated virus retains at least some capacity to elicit an immune response following administration to an immunocompetent subject. In some cases, an attenuated virus is capable of eliciting a protective immune response without causing any signs or symptoms of infection. Other embodiments provide the ability of an attenuated virus to cause disease or pathology in a subject that is reduced by at least 5% (e.g., 10%, 25%, 50%, 75%, 80%, 85%, 90%, 95%, etc.) or greater, relative to the ability of a wild-type virus to cause disease or pathology in the subject.

A “codon-optimized” nucleic acid (or polynucleotide) refers to a nucleic acid sequence that has been altered such that the codons are optimal for expression in a particular system (such as, e.g., a particular species, group of species, etc.). For example, a nucleic acid sequence can be optimized for expression in mammalian cells. Codon optimization does not alter the amino acid sequence of the encoded protein.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law, where the term is open-ended, allowing for the presence of more than that which is recited so long as basic or novel characteristics of that which is recited is not changed by the presence of more than that which is recited, and excludes prior art embodiments.

“Detect” refers to identifying the presence, absence, or amount of a substance (e.g., analyte, compound, agent, etc.) to be detected. By “detectable label” is meant an agent that, when linked to a molecule of interest, renders the latter detectable (e.g., via spectroscopic, photochemical, biochemical, immunochemical, chemical means, etc.). Nonlimiting examples of useful detectable labels include radioactive isotopes, magnetic beads, metallic beads, colloidal particles, fluorescent dyes, electron-dense reagents, enzymes (for example, as commonly used in an ELISA), biotin, digoxigenin, haptens, etc.

By “disease” is meant any condition, disorder, pathology, or the like that damages or interferes with the normal function of a cell, tissue, organ, or the like. In this disclosure, examples of diseases include those caused by Dengue virus infection and the symptoms and adverse effects that are caused by, either directly or indirectly, infection of the body with the Dengue virus. Dengue virus may induce a classical form of dengue fever (DF) and the symptoms thereof, characterized by, for example, without limitation, high fever, headache, stomachache, rash, myalgia, arthralgia, etc. Severe dengue, also known as Dengue Hemorrhagic Fever (DHF), and Dengue Shock Syndrome (DSS) may also be caused by dengue virus infection, where severe dengue and DSS are accompanied by thrombocytopenia, vascular leakage, hypotension, etc. DSS, which can be fatal, is characterized by systemic shock. Severe dengue or DHF are known to spread among individuals and populations and cause severe dengue epidemics.

By “effective amount” is meant the amount of an active therapeutic agent (e.g., composition, compound, biologic (e.g., a vaccine, peptide, polypeptide, polynucleotide, etc.), etc.) required to ameliorate, reduce, improve, abrogate, diminish, eliminate, or the like, the symptoms and/or effects of a disease, condition, or pathology in a subject suffering from the symptoms and/or effects of a disease, condition, or pathology relative to an untreated subject. The effective amount of an immunogen or a composition comprising an immunogen, as used to practice the methods of therapeutic treatment of disease, condition, or pathology caused by the Dengue virus, varies depending upon the manner of administration, the age, body weight, and general health of the subject. Ultimately, the attending physician or veterinarian will decide the appropriate amount and dosage regimen. Such amount is referred to as an “effective” amount.

A “therapeutically effective amount” refers to a quantity of a specified agent sufficient to achieve a desired effect in a subject being treated with that agent. For example, this may be the amount of a Dengue virus immunogen or vaccine useful for eliciting an immune response in a subject and/or for preventing infection by Dengue virus. Ideally, in the context of the present disclosure, a therapeutically effective amount of a Dengue virus vaccine or an anti-Dengue virus immunogenic composition is an amount sufficient to increase resistance to, prevent, ameliorate, reduce, and/or treat infection caused by Dengue virus in a subject without causing a substantial cytotoxic effect in the subject, where the benefits outweigh the detriments. The effective amount of an Dengue virus vaccine or Dengue virus immunogenic composition useful for increasing resistance to, preventing, ameliorating, reducing, and/or treating infection in a subject depends on, for example, the subject being treated, the manner of administration of the therapeutic composition and other factors, as noted supra.

By “fragment” is meant a portion of a polypeptide or nucleic acid molecule. This portion contains, preferably, at least 5% (e.g., 10%, 20%, 30%, 40%, 50%, 60%, 70%, 80%, 90%, etc.) of the entire length of the reference polypeptide or nucleic acid molecule. A fragment may contain 10 (e.g., 20, 30, 40, 50, 60, 70, 80, 90, or 100, 200, 300, 400, 500, 600, 700, 800, 900, 1000, etc.) or more nucleotides or amino acids. A portion or fragment of a polypeptide may be a peptide. In the case of an antibody or immunoglobulin fragment, the fragment typically binds to the target antigen. In another embodiment, the fragment may be sufficiently large to elicit an immune response in a subject and/or for preventing infection by Dengue virus.

By “fusion protein” is meant a protein generated by expression of a nucleic acid (polynucleotide) sequence engineered from nucleic acid sequences encoding at least a portion of two different (heterologous) proteins or peptides. To create a fusion protein, the nucleic acid sequences must be in the same reading frame and contain no internal stop codons. For example, a fusion protein includes Dengue virus protein fused to a heterologous protein, or fragments of Dengue virus protein fused to another Dengue virus protein (e.g., DENV prM/E, etc.).

By “genetic vaccine” is meant an immunogenic composition comprising a polynucleotide encoding an antigen,” such as for example, a Dengue virus antigen.

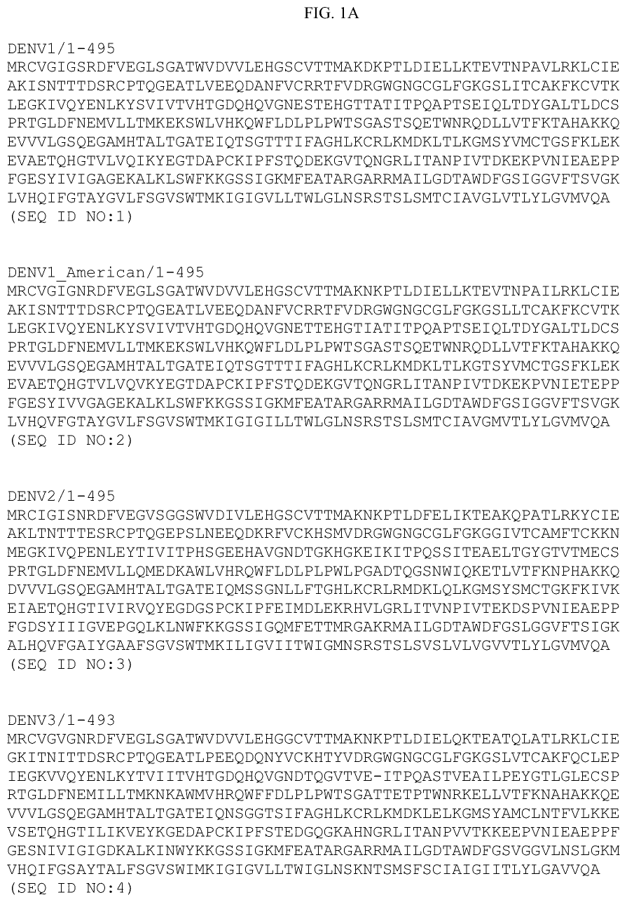

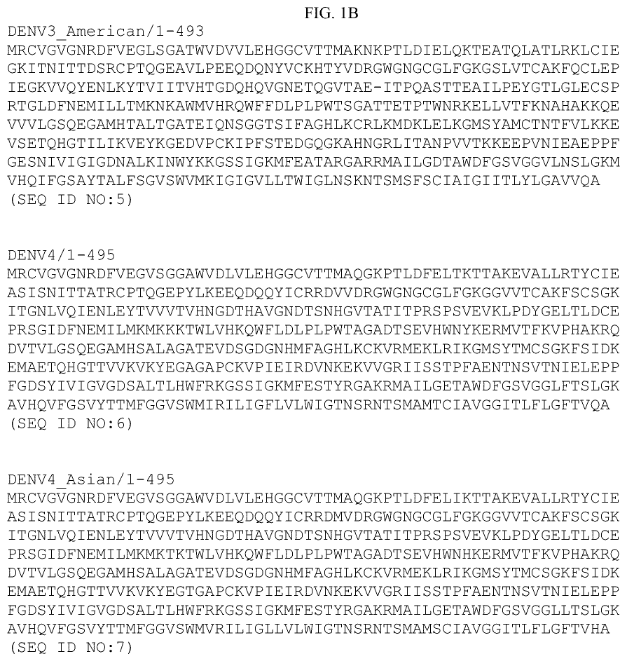

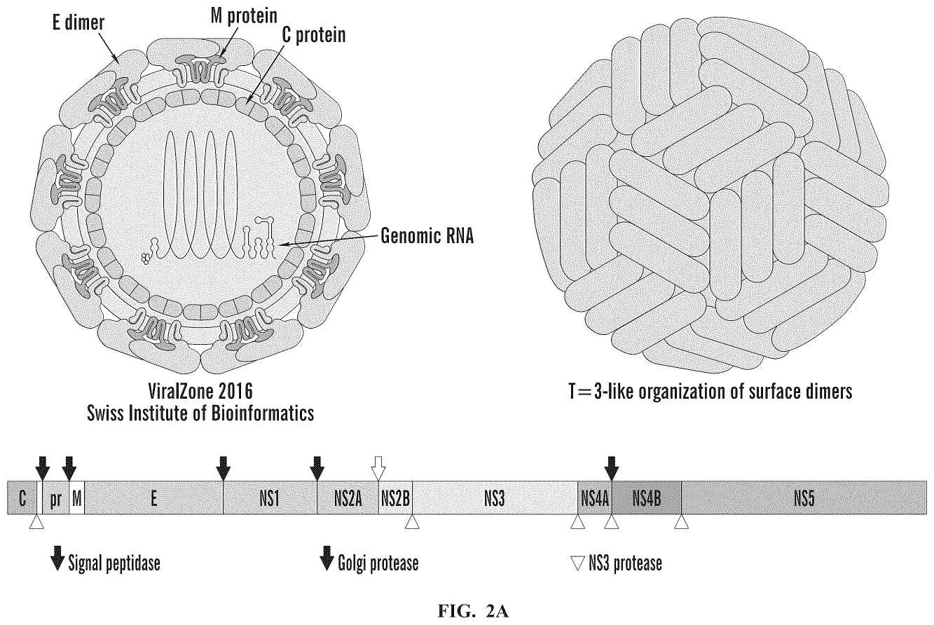

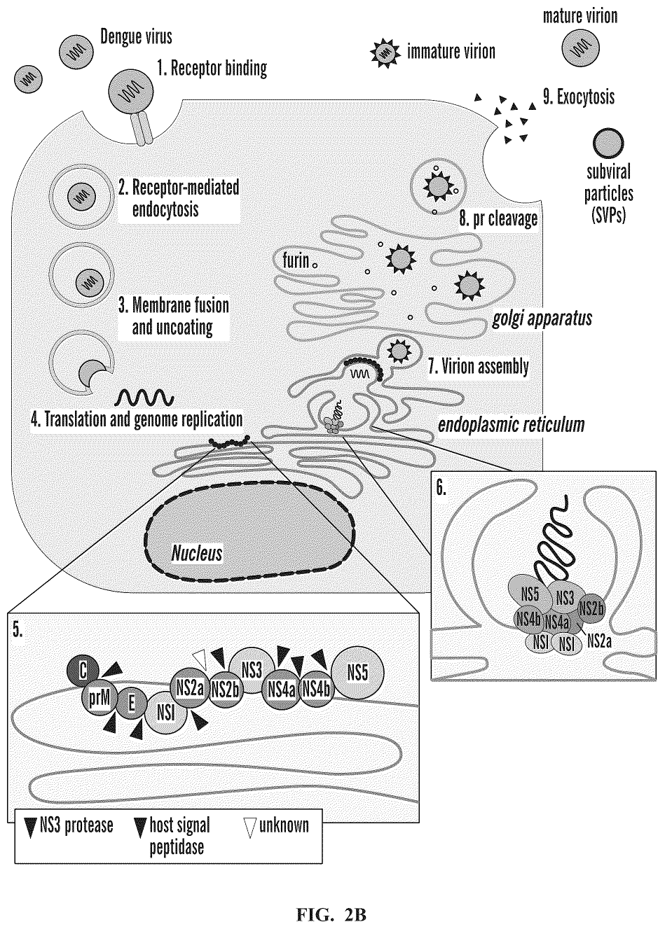

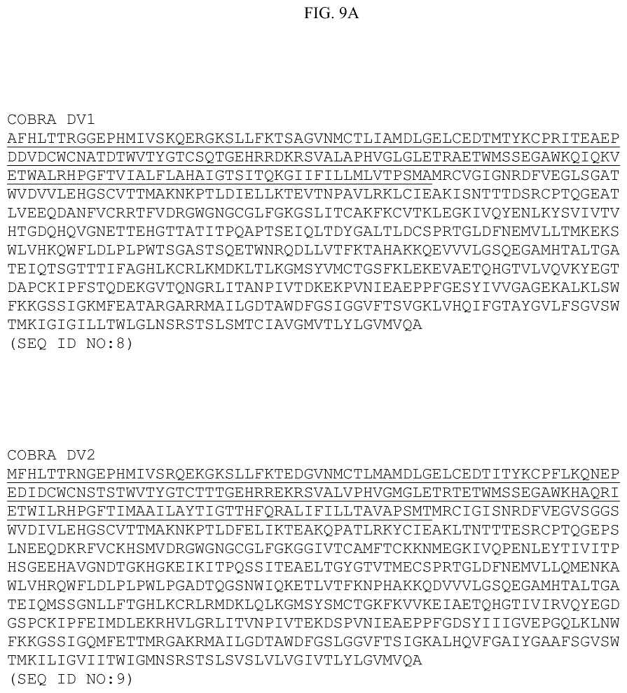

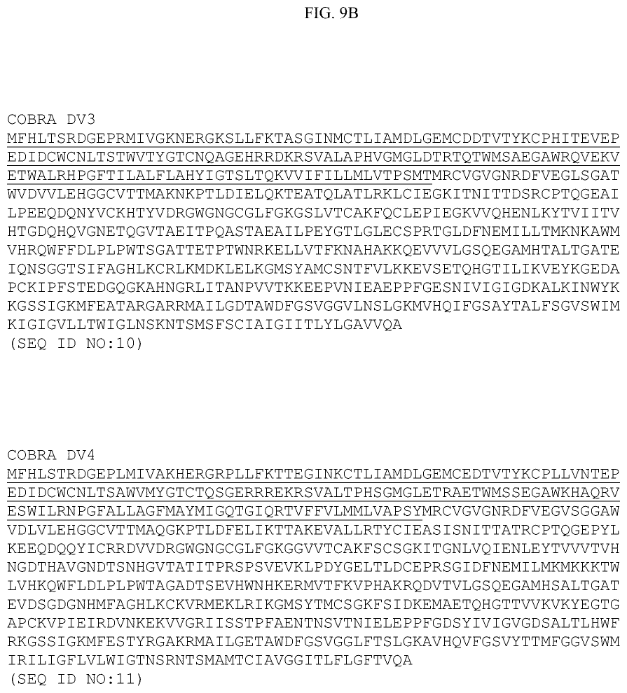

By “Dengue virus polypeptide” is meant an amino acid sequence that is at least 70% (e.g., 75%, 80%, 85%, 90%, 95%, 97%, 99%, etc.) identical to an amino acid sequence of a Dengue virus antigen as set forth in FIG. 1A (SEQ ID NOs:1-4), FIG. 1B (SEQ ID NOs: 5-7), FIG. 9A (SEQ ID NOs:8-9), FIG. 9B (SEQ ID NOs:10-11), any fragment thereof capable of inducing an immune response in an immunized subject. In one embodiment, a Dengue virus polypeptide comprises or consists of a DENV1, DENV2, DENV3, or DENV4 sequence, any fragment thereof, or any combinations of sequences or fragments thereof.

By “Dengue virus polynucleotide” is meant a nucleic acid molecule encoding an Dengue virus polypeptide (e.g., antigen, antigen protein, etc.), any fragment thereof, or any combinations of sequences or fragments thereof. The Dengue virus polynucleotide having a nucleic acid sequence that is at least 80% (e.g., 85%, 90%, 95%, 97%, 99%, etc.) identical to a nucleic acid sequence encoding an amino acid sequence that is at least 70% (e.g., 75%, 80%, 85%, 90%, 95%, 97%, 99%, etc.) identical to an amino acid sequence of a Dengue virus antigen as set forth in FIG. 1A (SEQ ID NOs: 1-4), FIG. 1B (SEQ ID NOs: 5-7), FIG. 9A (SEQ ID NOs: 8-9), FIG. 9B (SEQ ID NOs:10-11), or a fragment thereof capable of inducing an immune response in an immunized subject. Nucleic acid sequences encoding these Dengue virus polypeptides, fragments thereof, or combinations of sequences or fragments thereof, can be used to generate virus-like particles (VLPs) containing the Dengue protein antigens, which are used as immunogens or vaccines to generate an immune response, such as but not limited to, producing neutralizing antibodies and/or T-lymphocytes (or activated T-cells) in immunized subjects.

The terms “geographical location or geographical region” refers to preselected divisions of geographical areas of the earth, for example, by continent or other preselected territory or subdivision (e.g., the Middle East, which spans more than one continent, etc.). Examples of different geographical regions include countries (e.g., Costa Rico, Turkey, Egypt, Iraq, Azerbaijan, China, United States, etc.); continents (e.g., Asia, Europe, North America, South America, Oceania, Africa, etc.); recognized geopolitical subdivisions (such as the Middle East); or hemispheres of the world (e.g., Northern, Southern, Eastern, or Western hemispheres).

“Hybridization” means hydrogen bonding, which may be Watson-Crick, Hoogsteen, or reversed Hoogsteen hydrogen bonding, between complementary nucleobases. For example, in DNA, adenine and thymine, and cytosine and guanine, are, respectively, complementary nucleobases that pair through the formation of hydrogen bonds.

By “immunogen” is meant a substance (e.g., compound, agent, composition, etc.) which is capable, under appropriate conditions, of eliciting or stimulating an immune response, such as, but not limited to, the production of antibodies, a T-cell response, etc. in a subject (e.g., an animal), with such substance (e.g., compound, agent, composition, etc.) that is administered (e.g., delivered, injected, absorbed, etc.) into the subject.

The term “immune response” is meant any response mediated by an immunoresponsive cell. In one example of an immune response, leukocytes are recruited to carry out a variety of different specific functions in response to exposure to an antigen (e.g., a foreign entity, etc.). Immune responses are multifactorial processes that differ depending on the type of cells involved. Immune responses include cell-mediated responses (e.g., T cell responses, etc.), humoral responses (e.g., B cell/antibody responses, etc.), innate responses, and combinations thereof.

By “immunogenic composition” is meant a composition comprising an antigen (e.g., antigen sequence, immunogen, etc.) where the composition elicits an immune response in an immunized subject. As used herein, an “immunogenic composition” may be a composition comprising an immunogen (e.g., Dengue virus polypeptide, fragment thereof, etc.), such as for example, but not limited to, a vaccine comprising a Dengue virus polypeptide or fragment thereof, or medicinal product comprising a Dengue virus polypeptide, etc. In one embodiment, the immunogenic composition is a Dengue virus immunogenic composition comprising a Dengue virus antigen or immunogen (e.g., DENV1 (SEQ ID NOs:1, 2, 8), DENV2 (SEQ ID NOs:3, 9), DENV3 (SEQ ID NOs:4, 5, 10), DENV4 (SEQ ID NOs:6, 7, 11), any fragments thereof, any combinations thereof, etc.).

As will be appreciated by the skilled person in the art, if administered to a subject in need prior to the subject having contracted a disease or experiencing full-blown disease, an immunogenic composition can be prophylactic and result in the subject eliciting an immune response (e.g., a neutralizing antibody, cellular immune response, T-cell response, etc.) to protect against the disease, or to prevent a more severe case of the disease or condition, and/or the symptoms thereof. If administered to a subject in need following the subject having contracted the disease, an immunogenic composition can be therapeutic and result in the subject eliciting an immune response (e.g., a neutralizing antibody, cellular immune response, T-cell response, etc.) to treat the disease (e.g., by reducing, diminishing, abrogating, ameliorating, eliminating, etc. the disease, and/or symptoms thereof). In an embodiment, the immune response is a B cell response, which results in the production of antibodies (e.g., neutralizing antibodies, etc.) directed against the immunogen or immunogenic composition comprising the antigen or antigen sequence (e.g., DENV1 (SEQ ID NOs:1, 2, 8), DENV2 (SEQ ID NOs:3, 9), DENV3 (SEQ ID NOs:4, 5, 10), DENV4 (SEQ ID NOs:6, 7, 11), any fragments thereof, any combinations thereof, etc.). In a manner similar to the foregoing, in some embodiments, an immunogenic composition (e.g., vaccine, medicinal product, etc.) can be prophylactic. In some embodiments, an immunogenic composition (e.g., vaccine, medicinal product, etc.) can be therapeutic. In another embodiment, the immunogenic composition for prophylactic and/or therapeutic use comprises a Dengue virus immunogen (e.g., Dengue virus substance, compound, agent, composition, etc.), where the Dengue virus immunogen comprises any sequence set forth in FIG. 1A, FIG. 1B, FIG. 9A, FIG. 9B, DENV1 (SEQ ID NOs:1, 2, 8), DENV2 (SEQ ID NOs:3, 9), DENV3 (SEQ ID NOs:4, 5, 10), DENV4 (SEQ ID NOs:6, 7, 11), any fragments thereof, or any combinations thereof. In an embodiment, the disease is dengue fever (DF), characterized by high fever, headache, stomachache, rash, myalgia, and arthralgia, severe dengue, also known as Dengue Hemorrhagic Fever (DHF), or Dengue Shock Syndrome (DSS). The aforementioned diseases caused by Dengue virus infection are accompanied by thrombocytopenia, vascular leakage, hypotension, etc.

The term “immunize” (or immunization) refers to rendering a subject protected from a disease, infectious disease, or pathology, or the symptoms thereof, caused by the disease (e.g., Dengue virus), such as by vaccination with an antigen for the disease sufficient to stimulate an immune response (e.g., cell-mediated response, T-cell response, humoral response, B-cell response, antibody response, innate response, etc.).

By “inhibitory nucleic acid” is meant a double-stranded RNA, siRNA, shRNA, or antisense RNA, or a portion thereof, or a mimetic thereof, etc. that when administered to a mammalian cell results in a decrease (e.g., by 5%, 10%, 25%, 50%, 75%, 90-100%, etc.) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” substance (e.g., protein, peptide, nucleic acid, fragment thereof, etc.) is sufficiently free of other materials such that any impurities do not materially affect the biological properties of the substance or cause other adverse consequences. That is, a substance (e.g., nucleic acid, protein, peptide, fragment thereof, etc.) is purified if it is substantially free of cellular material, debris, non-relevant viral material, culture medium, etc. when produced by various techniques (e.g., recombinant DNA techniques, of chemical precursors or other chemicals when chemically synthesized, etc.). Purity and homogeneity are typically determined using standard purification methods and analytical chemistry techniques, for example, polyacrylamide gel electrophoresis or high-performance liquid chromatography. The term “purified” can denote, for example, that a nucleic acid or protein gives rise to essentially one band in an electrophoretic gel. For a protein that can be subjected to modifications, for example, phosphorylation or glycosylation, different modifications may give rise to different isolated proteins, which can be separately purified. The term “isolated” also embraces recombinant nucleic acids, proteins, viruses, etc., as well as chemically synthesized nucleic acids or peptides.

By “isolated polynucleotide” is meant a nucleic acid (e.g., a DNA molecule, etc.) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA a genomic fragment, cDNA fragment, etc. produced by PCR, restriction endonuclease digestion, a similar technique, etc.) independent of other sequences. In addition, the term includes an RNA molecule that is transcribed from a DNA molecule, as well as a recombinant DNA that is part of a hybrid gene encoding additional polypeptide sequence. In some embodiments, the isolated polynucleotide is a polynucleotide that encodes a Dengue virus polypeptide or fragment thereof.

By an “isolated polypeptide” is meant a polypeptide of the disclosure that has been separated from components that naturally accompany it. Typically, the polypeptide is isolated when it is at least 40%, (e.g., 50%, 60%, 70%, etc.), by weight, free from the proteins and naturally-occurring organic molecules with which it is naturally associated. In one embodiment, an isolated polypeptide preparation is at least 75% (e.g., 80%, 90%, 95%, 97%, 99%, etc.), by weight, free from the proteins and naturally-occurring organic molecules with which it is naturally associated. An isolated polypeptide may be obtained, for example, by extraction from a natural source; by expression of a recombinant nucleic acid encoding such a polypeptide; or by chemically synthesizing the protein. Purity can be measured by any standard, appropriate method, for example, column chromatography, polyacrylamide gel electrophoresis, by HPLC analysis, etc. An isolated polypeptide can refer to a broadly active virus immunogen polypeptide generated by the methods described herein.

By “linker” is meant one or more amino acids that serve as a spacer between two polypeptides or peptides of a fusion protein.

By “marker” is meant any protein or polynucleotide having an alteration in expression level or activity that is associated with a disease, condition, pathology, or disorder.

As used herein, “obtaining” as in “obtaining an agent” includes synthesizing, isolating, purchasing, etc., otherwise acquiring, for example, the agent.

The term “operably linked” refers to nucleic acid sequences as used herein. By way of example, a first nucleic acid sequence is operably linked to a second nucleic acid sequence when the first nucleic acid sequence is placed in a functional relationship with the second nucleic acid sequence. For instance, a promoter is operably linked to a coding sequence if the promoter affects (or allows) the transcription or expression of the coding sequence. Generally, operably linked DNA sequences are contiguous and, where necessary to join two protein-coding regions, are in the same reading frame.

In some embodiments, the nucleotide sequence encoding a Dengue virus protein (e.g., antigen, protein fragment, etc.) generated by the described methods can be optimized for expression in mammalian cells via codon-optimization and RNA optimization (such as, for example, to increase RNA stability) using procedures and techniques practiced in the art.

A broadly reactive, pan-epitopic immunogen, such as a Dengue virus protein, for eliciting an immune response in a subject possesses a collective set of strongly immunogenic epitopes (also called antigenic determinants). A Dengue virus protein described herein is a “pan-epitopic” immunogen that is suitable for use as a vaccine, which elicits a broadly reactive immune response, e.g., a neutralizing antibody response, etc. against a plurality of Dengue virus subtypes which express proteins on the viral surface (e.g., envelope (E) protein, etc.), when introduced into a host subject, in particular, a human subject infected with Dengue virus. The immunogenic antigen (e.g., vaccine, etc.) is advantageous for providing an anti-Dengue virus immunogen (e.g., a vaccine, etc.) that elicits a broadly active immune response against Dengue virus antigens with antigenic variability and similarity, and treats or protects against infection and disease caused by more than one Dengue virus type.

By “open reading frame (ORF)” is meant a series of nucleotide triplets (codons) that code for amino acids without any termination codons. These sequences are usually translatable into a peptide or polypeptide. In one embodiment, the amino acid sequence, peptide, or polypeptide is a Dengue virus sequence, peptide, or polypeptide.

As used herein, a Dengue virus “outbreak” refers to a collection of Dengue viruses, subtypes, strains, or isolates from within a geographical location (e.g., within a single country, a region, a locale, etc.) in a given time period (e.g., in a season, week, month, year, span of seasons, weeks, months, years, etc.).

The term “pharmaceutically acceptable vehicle” refers to conventional carriers (e.g., vehicles, diluents, excipients, mediums, etc.) that are physiologically and pharmaceutically acceptable for use, particularly in mammalian (e.g., human, animal, etc.) subjects. Such pharmaceutically acceptable vehicles are known to the skilled practitioner in the pertinent art and can be readily found in Remington's Pharmaceutical Sciences, by E. W. Martin, Mack Publishing Co., Easton, Pa., 15th Edition (1975) and its updated editions, which describes compositions and formulations suitable for pharmaceutical delivery of one or more therapeutic or immunogenic compositions, such as, for example, one or more Dengue virus vaccines, medicinal products, etc., and additional pharmaceutical agents. In general, the nature of a pharmaceutically acceptable carrier depends on the particular mode of administration being employed. For instance, parenteral formulations usually comprise injectable fluids/liquids that include pharmaceutically and physiologically acceptable fluids such as water, physiological saline, balanced salt solutions, aqueous dextrose, glycerol or the like as a vehicle. For solid compositions (for example, powder, pill, tablet, capsule forms, etc.), conventional non-toxic solid carriers may include, for example, pharmaceutical grades of mannitol, lactose, starch, magnesium stearate, etc., which typically stabilize and/or increase the half-life of a composition, drug, immunogen, etc. In addition to biologically-neutral carriers, pharmaceutical compositions to be administered can contain minor amounts of non-toxic auxiliary substances, such as, but not limited to, wetting or emulsifying agents, preservatives, pH buffering agents, and the like, for example sodium acetate or sorbitan monolaurate.

By “plasmid” is meant a circular nucleic acid molecule capable of autonomous replication in a host cell.

By “polypeptide” (or protein) is meant a polymer in which the monomers comprise amino acid residues that are joined together through amide bonds. When the amino acids are alpha-amino acids, either the L-optical isomer or the D-optical isomer can be used. The terms “polypeptide” or “protein” as used herein are intended to encompass any amino acid sequence and include modified sequences such as glycoproteins. The term “polypeptide” is specifically intended to cover naturally occurring proteins, as well as those which are recombinantly or synthetically produced. The term “residue” or “amino acid residue” also refers to an amino acid that is incorporated into a protein, polypeptide, peptide, etc.

Conservative amino acid substitutions are those substitutions that, when made, least interfere with the properties of the original protein, where the structure and especially the function of the protein is conserved and is not significantly changed by such amino acid substitutions. Examples of conservative amino acid substitutions are known in the art, e.g., as set forth in, for example, U.S. Publication No. 2015/0030628. Conservative substitutions generally maintain (a) the structure of the polypeptide backbone in the area of the substitution, for example, as a sheet or helical conformation; (b) the charge or hydrophobicity of the molecule at the target site; and/or (c) the bulk of the side chain. The substitutions that are generally expected to produce the greatest changes in protein properties are non-conservative, for instance, changes in which (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histadyl, is substituted for (or by) an electronegative residue, for example, glutamyl or aspartyl; or (d) a residue having a bulky side chain, for example, phenylalanine, is substituted for (or by) one not having a side chain, for example, glycine.

“Primer set” means a set of oligonucleotides that may be used, for example, for PCR. A primer set would consist of at least 2 (e.g., 4, 6, 8, 10, 12, 14, 16, 18, 20, 30, 40, 50, 60, 80, 100, 200, 250, 300, 400, 500, 600, etc.) or more primers.

By “promoter” is meant an array of nucleic acid control sequences, which direct transcription of a nucleic acid. A promoter includes necessary nucleic acid sequences near the start site of transcription. A promoter also optionally includes distal enhancer or repressor sequence elements. A “constitutive promoter” is a promoter that is continuously active and is not subject to regulation by external signals or molecules. In contrast, the activity of an “inducible promoter” is regulated by an external signal or molecule (for example, a transcription factor, etc.). By way of example, a promoter may be a CMV promoter.

As will be appreciated by the skilled practitioner in the art, the term “purified” does not require absolute purity; rather, it is intended as a relative term. Thus, for example, a purified peptide, protein, virus, or other active compound is one that is isolated in whole or in part from naturally associated proteins and other contaminants. In certain embodiments, the term “substantially purified” refers to a peptide, protein, virus or other active compound that has been isolated from a cell, cell culture medium, or other crude preparation and subjected to routine methods, such as fractionation, chromatography, or electrophoresis, to remove various components of the initial preparation, such as proteins, cellular debris, and other components.

A “recombinant” nucleic acid, protein or virus is one that has a sequence that is not naturally occurring or that has a sequence that is made by an artificial combination of two otherwise separated segments of sequence. Such an artificial combination is often accomplished by chemical synthesis or by the artificial manipulation of isolated segments of nucleic acids, for example, by genetic engineering techniques. A “non-naturally occurring” nucleic acid, protein or virus is one that may be made via recombinant technology, artificial manipulation, or genetic or molecular biological engineering procedures and techniques, such as those commonly practiced in the art.

By “reduces” is meant a negative alteration of at least 5% (e.g., 10%, 25%, 30%, 40%, 50%, 75%, 80%, 85%, 90%, 95%, 98%, 100%, etc.).

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, the complete cDNA or gene sequence, a polypeptide sequence or fragment thereof, such as a peptide, etc. For polypeptides, the length of the reference polypeptide sequence will generally be at least 16 amino acids (e.g., 20 amino acids, 25 amino acids, 35 amino acids, 50 amino acids, 100 amino acids, etc., or any integer therebetween). For nucleic acids, the length of the reference nucleic acid sequence will generally be at least 50 nucleotides (e.g., 60 nucleotides, 75 nucleotides, 100 nucleotides, 300 nucleotides, etc., or any integer therebetween).

By “specifically binds” is meant a compound or antibody that recognizes and binds a polypeptide, such as a virus polypeptide, peptide, or vaccine product, but which does not substantially recognize and bind other molecules in a sample, for example, a biological sample, which naturally includes a polypeptide, such as a virus polypeptide or peptide.

Nucleic acid molecules useful in the methods described in various embodiments herein include any nucleic acid molecule that encodes a polypeptide as described, or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pairing to form a double-stranded molecule between complementary polynucleotide sequences (e.g., a gene), or portions thereof, under various conditions of stringency. (See, e.g., Wahl, G. M. and S. L. Berger, (1987), Methods Enzymol., 152:399; Kimmel, A. R. (1987), Methods Enzymol. 152:507).

By way of example, stringent hybridization conditions include a stringent salt concentration ordinarily less than 750 mM NaCl and 75 mM trisodium citrate (e.g., 500 mM NaCl and 50 mM trisodium citrate, 250 mM NaCl and 25 mM trisodium citrate, etc.). Low stringency hybridization can be obtained in the absence of organic solvent, e.g., formamide, etc., while high stringency hybridization can be obtained in the presence of at least 35% formamide (e.g., 50% formamide, etc.). Stringent temperature conditions will ordinarily include temperatures of at least 30° C. (e.g., 37° C., 42° C., etc.). Varying additional parameters, such as hybridization time, the concentration of detergent, e.g., sodium dodecyl sulfate (SDS), etc., and the inclusion or exclusion of carrier DNA, are well known to those skilled in the art. Various levels of stringency are accomplished by combining these various conditions as needed. In a one embodiment, hybridization will occur at 30° C. in 750 mM NaCl, 75 mM trisodium citrate, and 1% SDS. In another embodiment, hybridization will occur at 37° C. in 500 mM NaCl, 50 mM trisodium citrate, 1% SDS, 35% formamide, and 100 μg/ml denatured salmon sperm DNA (ssDNA). In a further embodiment, hybridization will occur at 42° C. in 250 mM NaCl, 25 mM trisodium citrate, 1% SDS, 50% formamide, and 200 μg/ml ssDNA. Useful variations of these conditions will be apparent to those skilled in the art.

For most applications, washing steps that follow hybridization may also vary in stringency. Wash stringency conditions can be defined by salt concentration and by temperature. As above, wash stringency can be increased by decreasing salt concentration or by increasing temperature. For example, stringent salt concentration for the wash steps may be less than 30 mM NaCl and 3 mM trisodium citrate (e.g., 15 mM NaCl and 1.5 mM trisodium citrate, etc.). Stringent temperature conditions for the wash steps will ordinarily include a temperature of at least 25° C. (e.g., 42° C., 68° C., etc.). In another embodiment, wash steps may occur at 25° C. in 30 mM NaCl, 3 mM trisodium citrate, and 0.1% SDS. In a further embodiment, wash steps may occur at 42 C in 15 mM NaCl, 1.5 mM trisodium citrate, and 0.1% SDS. In yet another embodiment, wash steps may occur at 68° C. in 15 mM NaCl, 1.5 mM trisodium citrate, and 0.1% SDS. Additional variations of these conditions will be apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting 50% or greater identity to a reference amino acid sequence (for example, any one of the amino acid sequences described herein) or nucleic acid sequence (for example, any one of the nucleic acid sequences described herein). In other embodiments, such a sequence has 60% (e.g., 80%, 85%, 90%, 95%, 99%, etc.) or greater identity to the amino acid or nucleic acid sequence used for comparison.

“Sequence identity” refers to the similarity between amino acid or nucleic acid sequences that is expressed in terms of the similarity between the sequences. Sequence identity is frequently measured in terms of percentage identity (or similarity or homology); the higher the percentage, the more similar the sequences are. Homologs or variants of a given gene or protein will possess a relatively high degree of sequence identity when aligned using standard methods. Sequence identity is typically measured using sequence analysis software (e.g., Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs, etc.). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence. In addition, other programs and alignment algorithms are described in, for example, Smith and Waterman, 1981, Adv. Appl. Math. 2:482; Needleman and Wunsch, 1970, J Mol. Biol. 48:443; Pearson and Lipman, 1988, Proc. Natl. Acad. Sci. U.S.A. 85:2444; Higgins and Sharp, 1988, Gene 73:237-244; Higgins and Sharp, 1989, CABIOS 5:151-153; Corpet et al., 1988, Nucleic Acids Research 16:10881-10890; Pearson and Lipman, 1988, Proc. Natl. Acad. Sci. U.S.A. 85:2444; and Altschul et al., 1994, Nature Genet. 6:119-129, etc. The NCBI Basic Local Alignment Search Tool (BLAST™) (Altschul et al. 1990, J Mol. Biol. 215:403-410) is readily available from several sources, including the National Center for Biotechnology Information (NCBI, Bethesda, MD) and on the Internet, for use in connection with the sequence analysis programs blastp, blastn, blastx, tblastn and tblastx.

By “subject” is meant an animal, e.g., a mammal, including, but not limited to, a human, a non-human primate, or a non-human mammal, such as, but not limited to a bovine, equine, canine, ovine, feline, sheep, goat, llama, camel, rodent (e.g., rat, mouse, gerbil, hamster, bats, etc.), monkeys, apes, etc., or any animal which may be located in an area or region susceptible to mosquito-infection, such as but not limited to, those climates which are wet, hot, and humid where mosquitoes would thrive, tropical or subtropical regions, areas where standing water is present, etc. In a non-limiting example, a subject is one who is infected with a Dengue virus, or who is at risk of infection by such virus, or who is susceptible to such infection. In particular aspects as described herein, the subject is a human subject, such as a patient, a traveler of affected areas, a person living in a region susceptible to Dengue virus, etc.

Ranges provided herein are understood to be shorthand for all of the values within the range, inclusive of the first and last stated values. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, etc. In some embodiments, 1 to 50 may consist of any numbers including 1 and 50, numbers therebetween, or greater, consecutively, such as, for example, to 100 or greater.

As used herein, the terms “treat,” treating,” “treatment,” and the like refer to reducing, diminishing, decreasing, abrogating, ameliorating, or eliminating, a disease, condition, disorder, or pathology, and/or symptoms associated therewith. While not intending to be limiting, “treating” typically relates to a therapeutic intervention that occurs after a disease, condition, disorder, or pathology, and/or symptoms associated therewith, have begun to develop to reduce the severity of the disease, etc., and the associated signs and symptoms. It will be appreciated that, although not precluded, treating a disorder or condition does not require that the disease, condition, disorder, pathology, or the symptoms associated therewith, be completely eliminated.

As used herein, the terms “prevent,” “preventing,” “prevention,” “prophylactic treatment” and the like, refer to inhibiting or blocking a disease state, or the full development of a disease in a subject, or reducing the probability of developing a disease, disorder or condition in a subject, who does not have, but is at risk of developing, or is susceptible to developing, a disease, disorder, or condition.

As referred to herein, a “transformed” cell is a cell into which a nucleic acid molecule or polynucleotide sequence has been introduced by molecular biology techniques. As used herein, the term “transformation” encompasses all techniques by which a nucleic acid molecule or polynucleotide may be introduced into such a cell, including transfection with viral vectors, transformation with plasmid vectors, and introduction of naked nucleic acid (e.g., DNA RNA, etc.) by electroporation, lipofection, particle gun acceleration, and the like.

By “vaccine” is meant a preparation of immunogenic material (e.g., protein, nucleic acid, vaccine, etc.) capable of stimulating (or eliciting) an immune response, administered to a subject to treat a disease, condition, or pathology, or to prevent a disease, condition, or pathology, such as an infectious disease (caused by Dengue virus infection, for example). The immunogenic material may include, for example, attenuated or killed microorganisms (such as, for example, attenuated viruses), or antigenic proteins, peptides or DNA derived from such microorganisms. Vaccines may elicit a prophylactic (or preventative) immune response in the subject; they may also elicit a therapeutic response immune response in a subject. As mentioned above, methods of vaccine administration vary according to the vaccine, and can include routes or means, such as inoculation (intravenous or subcutaneous injection), ingestion, inhalation, or other forms of administration. Inoculations can be delivered by any number of routes, including parenteral, such as intravenous, subcutaneous, intramuscular, etc. Vaccines may also be administered with an adjuvant to boost the immune response.

As used herein, a “vector” refers to a nucleic acid (or polynucleotide) molecule into which foreign nucleic acid can be inserted without disrupting the ability of the vector to replicate in and/or integrate into a host cell. A vector can include nucleic acid sequences that permit it to replicate in a host cell, such as an origin of replication. An insertional vector is capable of inserting itself into a host nucleic acid. A vector can also include one or more selectable marker genes and other genetic elements. An expression vector is a vector that contains the necessary regulatory sequences to allow transcription and translation of inserted gene or genes in a host cell. In some embodiments of the present disclosure, the vector encodes Dengue virus protein. In some embodiments, the vector is the pTR600 expression vector (U.S. Patent Application Publication No. 2002/0106798; Ross et al., 2000, Nat Immunol. 1(2):102-103; and Green et al., 2001, Vaccine 20:242-248).

By “virus-like particle (VLP)” is meant virus particles, as disclosed herein, for example, Dengue virus VLPs, made up of one of more viral structural proteins, but lacking the viral genome. Because VLPs lack a viral genome, they are non-infectious and yield safer and potentially more-economical vaccines and vaccine products. In addition, VLPs can often be produced by heterologous expression and can be easily purified. Most VLPs comprise at least a viral core protein that drives budding and release of particles from a host cell. Dengue virus VLPs can be produced by transfection of host cells with plasmids encoding proteins derived from Dengue virus. After incubation of the transfected cells for an appropriate time to allow for protein expression (such as, for example, approximately 72 hours, etc.), VLPs can be isolated from cell culture supernatants. By way of example, a protocol for purifying or isolating Dengue VLPs from cell supernatants can involve low speed centrifugation (to remove cell debris), vacuum filtration and ultracentrifugation of the VLPs through 20% glycerol.

Unless specifically stated or obvious from context, as used herein, the term “or” is understood to be inclusive. Unless specifically stated or obvious from context, as used herein, the terms “a”, “an”, and “the” are understood to be singular or plural. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description.

Unless specifically stated or obvious from context, as used herein, it is understood that numerical values described herein are within a range of normal tolerance in the art, for example within 1 (e.g., 1.5, 2, 2.5, 3, etc.) standard deviations of the mean. For example, the numerical value may be within 10% (e.g., 9%, 8%, 7%, 6%, 5%, 4%, 3%, 2%, 1%, 0.5%, 0.1%, 0.05%, 0.01%, etc.) of the stated value. Unless otherwise clear from context, all numerical values provided herein are within a range of normal tolerance in the art.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Dengue Virus Antigens

Dengue viruses are old viruses that have re-emerged during the last half of the 20th century. Regarded as a tropical fever disease affecting more than two thirds of the world's population, dengue is the main cause, after malaria, of tropical fever among travelers. Dengue disease ranks as the most important mosquito-borne viral disease in the world. The lack of potent antiviral drugs and an effective vaccine results in approximately 500,000 individuals, mainly children, being hospitalized with severe dengue disease every year and causes serious economic losses for households and entire nations.

Featured herein are synthetic (non-naturally occurring), broadly reactive immunogenic antigens, e.g., protein and glycoprotein antigens, derived from Dengue virus (including, for example, different Dengue virus subtypes, different fragments of Dengue virus, combinations thereof, etc.), that elicit a potent, broadly reactive immune response in a subject, particularly, a human subject. Such immunogenic antigens are also referred to as “immunogens” herein.

Provided are immunogens that protect against disease caused by Dengue virus subtypes, spanning several years, including drifted strains not yet in existence. In an embodiment, fully synthetic Dengue protein antigens are featured. Such Dengue virus antigens are synthetic proteins not found in nature, yet they retain all of the functions of a natural Dengue viral protein and are immunogenic, i.e., they can elicit an immune response, in particular, a broadly reactive immune response in the form of neutralizing antibodies and/or reactive T lymphocytes, following administration or delivery to, or introduction into, a subject. Also provided are immunogenic compositions (e.g., vaccines, medicinal products, etc.) comprising the synthetic Dengue virus protein antigens, or nucleic acids encoding the antigens.

A Dengue virus antigen amino acid sequence and a protein antigen having such sequence are particularly for use as an immunogen, or in an immunogenic composition, e.g., a vaccine, that elicits a broadly reactive immune response in a subject, particularly a human subject, to whom the composition, or vaccine, is administered. The Dengue virus immunogens comprise antigenic determinants that represent different “antigenic spaces” that are derived from the sequences of the four Dengue virus subtypes analyzed based on time periods (e.g., seasonal periods of time, either overlapping or non-overlapping time periods (e.g., calendar time periods)) and/or geographical location. Such overlapping or non-overlapping time periods may encompass different intervals of time, for example, 5 months, 6 months, 7 months, eight months, nine months, 10 months, 11 months, 1 year, 2 years, 3 years, 4 years, 5 years, 6 years, 7 years, 8 years, 10 years or more, including time intervals therebetween.

The Dengue virus antigens described herein embrace seasonal, regional, pan-epitopic, broadly reactive antigens of Dengue virus and subtypes thereof, especially antigens containing sequences based on Dengue virus drift variants, wherein the antigens are designed to generate a broadly active immune response, particularly in the form of neutralizing antibodies, in a subject, particularly a human subject. Such antigens are beneficial as immunogens, which elicit an immune response (e.g., production of neutralizing antibodies) against the Dengue virus where multiple subtypes of Dengue co-circulate at one time.

The broadly reactive Dengue immunogenic antigens described herein are derived from sequences of all subtypes of Dengue virus, and, as a consequence, stimulate a competent immune system to generate antibodies against multi-subtype antigenic epitopes (e.g., determinants, etc.) on the Dengue virus antigens (e.g., DENV M polypeptide, DENY prM polypeptide, DENV envelope polypeptides, DENV prM/E polypeptide, etc., or fragments or combinations thereof, etc.) following infection. Thus, the synthetic Dengue virus antigens comprise amino acid (or polynucleotide) sequences that will elicit greater numbers of neutralizing antibodies against all four Dengue virus subtypes (and against drift variants within and across multiple years of Dengue virus infection) compared with wild-type Dengue antigen sequences.

A Dengue immunogenic protein, or immunogen, as described herein can be employed in an immunogenic composition or as a vaccine that may afford protection against many Dengue virus strains over several years. The broadly reactive Dengue virus immunogens and vaccines described herein are advantageous in that they are designed to provide broader and longer-lasting protection against several Dengue strains (or subtypes) prevalent in different geographical locations. Provided by the immunogens and their sequences as described herein is a universal and broad-spectrum Dengue virus vaccine that may alleviate the need for the administration of different immunogenic compositions (e.g., vaccines, medicinal products, etc.) against subtypes of Dengue virus that require repeated administration.

The immunogenic Dengue virus antigens described herein may be used in immunogenic compositions (e.g., Dengue vaccines, etc.) that afford protective immunity against infection and disease caused by one or more Dengue virus serotypes (or subtypes) in a subject. The protective immunity is provided in the subject through the elicitation of potent, broadly reactive, anti-Dengue virus protein specific antibody responses that protect the subject against the four Dengue virus serotypes, drifted, Dengue virus strains and pandemic Dengue virus strains. The immunogenic compositions and vaccines provide an advantage over prior and traditional immunogenic compositions and vaccines directed against Dengue virus, which typically depend on the selection of candidate vaccine viruses by public health authorities following analysis of data collected through active surveillance of Dengue viruses that periodically or episodically circulate in certain time periods.

Dengue Virus Structure

Dengue virus (DENV) is an enveloped, single-stranded, positive-sense RNA arbovirus. Its RNA genome consists of approximately 10,700 nucleotides and encodes a precursor polyprotein of 3,411 amino acids which contains three structural proteins, namely, the capsid (C), membrane (M), and envelope (E) proteins, which comprise the outer coat of the mature virus particle. The replication process of the viral genome does not involve the structural proteins. The Dengue virus envelope and precursor Membrane (prM) proteins are present on the surface of immature virions. When the prM and E proteins are co-expressed, they produce recombinant virus-like particles (VLPs). The prM protein contains 166 amino acids, and upon cleavage with furin or furin-like protease at position 91 of the prM protein, to produce the pr peptide and M protein. DENV also has seven non-structural (NS) proteins (e.g., NS1, NS2A, NS2B, NS3, NS4A, NS4B, and NS5). which are not present in mature virus particles at detectable levels, but are found in the infected cells. The nonstructural proteins are involved in replication of the viral genome and host immune evasion. (For a review of DENV, see, e.g., Back, A. T. et al., 2013, Infect Ecol Epidemiol., Vol. 3, published online 10.3402/iee.v3i0.19839).

The open reading frame of DENV is flanked by two untranslated regions (5′ and 3′ UTR) of approximately 95-135 and 114-650 nucleotides, respectively. The 5′-end contains a type I cap, similar to cellular mRNA, and the viral RNA (vRNA) is translated by a cap-dependent initiation scanning the 5′-UTR. The 3′-end of DENV lacks a poly(A) tail, but terminates in a conserved stem-loop (SL) structure. Both the 5′- and 3′-UTRs are required for efficient translation and replication. The UTRs have characteristic secondary structures that confer distinct functions and show high sequence conservation among different DENV serotypes (subtypes). The 5′-UTR contains a large stem-loop (SLA) that may act as the promoter for the viral RNA-dependent RNA polymerase (RdRp) NSS. Both the 5′- and the 3′-UTRs contain complementary Upstream AUG Regions (UAR) and cyclization sequences (CS) that hybridize in order to mediate genome cyclization and RNA synthesis.

The various steps in the flavivirus life cycle include virions binding to cell-surface attachment molecules and receptors, which are internalized through endocytosis. Due to the low pH of the endosome, viral glycoproteins mediate the fusion of viral and cellular membranes, allowing disassembly of the virion and release of vRNA into the cytoplasm. vRNA is translated into a polyprotein that is processed by viral and cellular proteases, and the viral NS proteins replicate the genome RNA. Virus assembly occurs at the endoplasmic reticulum (ER) membrane, where the C protein and vRNA are enveloped by the ER membrane and glycoproteins to form immature virus particles. Immature virus particles are transported through the Golgi secretory pathway, and in the acidic environment of the trans-Golgi network (TGN), furin-mediated cleavage of membrane preprotein prM drives maturation of the virus. Thereafter, mature virus is released from the cell.

Virus Entry

The entry of DENV into a host cell is mediated by receptor-mediated endocytosis through an as-yet unidentified cell-surface receptor. Candidate cellular receptors required for viral entry are various glycoproteins (i.e. heparin sulfates, etc.), dendritic cell-specific intercellular adhesion molecule-3-grabbing non-integrin (DC-SIGN), or a mannose receptor. The human C-type lectin-like molecule CLEC5A has been suggested to act as a critical macrophage receptor for DENV and has been described as a proinflammatory receptor for DENY that contributes to lethal disease in mice. The viral E glycoprotein (or envelope protein) is generally believed to affect host cell receptor binding, viral entry, and is a major target for humoral immunity. The E protein is composed of three domains: domain I, domain II (harboring the fusion peptide at its distal tip), and domain III (responsible for receptor-binding activity). In the mature state, the E protein exists as a homodimer with the fusion peptide inaccessible. Low pH-induced trimerization exposes the hydrophobic fusion peptide in a manner consistent with membrane fusion mediated by class II fusion proteins. Mutations of residues constituting the ligand pocket at the interface of domain I and II alter the required pH threshold and affect virulence. Of the two potential asparagine (N)-linked glycosylation sites at positions Asn-67 and Asn-153, the former is unique for DENVs and the latter is conserved in most flaviviruses. The E proteins of the DENV serotypes are differentially glycosylated, which may affect the antigenic properties of DENV.

Upon internalization, the acidic pH in the endosome triggers a conformational change in the E protein, which mediates fusion with the membrane. The viral nucleocapsid is released into the cytoplasm where DENV uncoats and releases its genome. The input positive-strand vRNA is translated into a single polyprotein, which is cleaved into the individual structural and NS proteins. The input strand translation is followed by a transition from translation to synthesis of a negative-strand intermediate, which serves as a template for new positive-strand vRNA. Multiple rounds of translation produce high levels of DENV proteins that together with vRNA are assembled into progeny virions.

Dengue Virus Structural Proteins

DENV infection induces intracellular membrane alterations in the cytosol of cells, which form vesicle packets (VPs) or smooth membrane structures (SMS) where the viral replication complex (RC) accumulates. The induction of membrane structures may serve as a scaffold for anchoring the viral RC. The C-terminal regions of the C, prM, and E proteins contain hydrophobic amino acids that serve as signal sequences for insertion of the remaining protein into the ER membrane. An ER signal peptidase together with the viral NS2B-NS3 protease cleaves the structural proteins and NS1 protein into individual membrane-bound proteins. The NS3 protein acts, together with its cofactor NS2B, as the viral serine protease needed for polyprotein-processing through its N-terminal end. This heterodimer protein complex cleaves on the cytoplasmic side of the ER membrane at the junctions between NS2A-NS2B, NS2B-NS3, NS3-NS4A, and NS4B-NSS, as well as on the internal sites within C, NS2A, NS3, and NS4A.

The C-terminal end of the DENY NS3 protein has three enzymatic properties: a 5′ RNA-triphosphatase (RTP), a nucleoside triphosphatase (NTPase), and a helicase. NS3, which forms a complex with NS5 and assists in viral replication through unwinding of RNA and dephosphorylation prior to capping at the 5′-end. The remaining NS proteins are cleaved by the viral serine protease NS3 that requires NS2B as a cofactor for catalytic activity. However, a host cell signal peptidase mediates post-translational modifications on the NS4A-4B proteins.

The NS1 glycoprotein has two glycosylation sites that are conserved among flaviviruses. NS1 is synthesized in the ER as a hydrophilic monomer, but it forms a hydrophobic homodimer. The NS1 dimer is transported to the Golgi apparatus where it undergoes carbohydrate trimming. NS1 is believed to facilitate viral infection and DENV pathogenesis, as it is secreted from infected cells (sNS1) and has been shown to be immunologically important. Antibodies raised against sNS1 proteins have been proposed to cause endothelial dysfunction due to cross-reactivity to host proteins and endothelial cells. sNS1 may be an important modulator of the complement pathway and may protect DENV from complement-dependent neutralization in solution.

NS2A, NS4A and NS4B are small hydrophobic proteins that are less well characterized, but may play an inhibitory role in interferon (IFN)-mediated signal transduction. Because the NS proteins are hydrophobic, they may be involved in proper localization of viral proteins and vRNA during replication and virion assembly. Formation of DENV-induced cytoplasmic membrane structures are believed to be an arrangement of the NS4A protein.

NS5 is the largest NS protein encoded in the DENV genome. The NS5 protein is approximately 103 kDa in length and has three major functional domains: the N-terminal S-adenosyl methionine methyltransferase (MTase), the nuclear localization sequences (NLS), and the RdRp activity in its C-terminal domain. The MTase spans amino acid residues 1 to 239 and is responsible for guanine N-7 and ribose 2′-O-methylations required for the capping of the DENV genome. The cap structure is recognized by the host cell translational machinery. The NLS (residues 320-405) interacts with the NS3 viral helicase and is recognized by cellular factors, allowing protein transport to the nucleus. The NS5 polymerase domain RdRp (residues 273-900) is responsible for synthesizing new vRNA genomes.

Prior to secretion of new viral particles, the third structural protein (pr)M is processed into the mature M protein by furin host protease. Briefly, the Dengue E protein is involved in the attachment of virions binding to cell-surface attachment molecules and receptors, which are internalized through endocytosis. Due to the low pH of the endosome, viral glycoproteins mediate the fusion of viral and cellular membranes, allowing disassembly of the virion and release of viral RNA (vRNA) into the cytoplasm. vRNA is translated into a polyprotein that is processed by viral and cellular proteases, and the viral NS proteins replicate the genome RNA. Virus assembly occurs at the endoplasmic reticulum (ER) membrane, where the C protein and vRNA are enveloped by the ER membrane and glycoproteins to form immature virus particles. Immature virus particles are transported through the Golgi secretory pathway, and in the acidic environment of the trans-Golgi network (TGN), furin- or furin-like-mediated cleavage of membrane preprotein prM drives maturation of the virus. It is believed that prM protects the E proteins from pH-induced reorganization and premature fusion during secretion; therefore, the maturation event is necessary for DENV infectivity. Thereafter, mature virus is released from the cell.

The Humoral Immune Response Against Dengue Virus

In infected subjects, the humoral immune response is hypothesized to be vital for controlling DENV infection and dissemination, and infection with one serotype provides long-lasting protection to that specific serotype (homotypic immunity). Subsequent infection by another DENV serotype results in short-lived protection (heterotypic immunity), and may eventually be harmful and increase the risk of severe dengue disease. The transient nature of heterotypic immunity is believed to be due to cross-reactive viral E protein-specific antibodies which are protective above a certain concentration threshold.

The principal targets of a natural antibody response to DENV infection in humans are the prM, the E structural proteins, and the NS1 protein. Weak antibody responses to other NS proteins, for example, NS3 and NSS, have also been detected. Neutralizing antibodies are directed against the viral E protein and inhibit viral attachment, internalization, and replication within cells. While multiple epitopes reside within each of the three E domains, the dimeric conformation of the E protein on the virion surface and its tightly packed mature form can affect the accessibility of the E protein to antibody binding. Complement activation resulting from host immune responses to dengue infection is a feature of severe dengue disease and is related to vascular leakage, which is associated with disease severity.

The DENV antigen sequences, immunogenic compositions and vaccines described herein induce a broadly reactive immune response (antibodies) that target and are directed against the proteins, e.g., the E protein, of multiple DENV subtypes, e.g., all of DENV1, DENV2, DENV3 and DENV4 E proteins.

The Cellular Immune Response Against Dengue Virus

Both humoral and cellular immune responses play important roles in dengue pathogenesis. DENV can infect both CD4+ T-cells and CD8+ T-cells, and cellular immune responses can be either protective or reactive. DENV-specific T-cells respond with a diverse set of effector functions, including proliferation, target cell lysis, and the production of a range of cytokines. CD4+ T-cells produce IFNγ, TNFα, TNFβ, interleukin-2 (IL-2), and CC-chemokine ligand 4 (CCL4; also known as MIP1β), which may contribute to pathogenesis. The production of T helper type-2 cytokines, such as IL-4, is less common. In uncomplicated DENV infections, relatively more CD8+ T-cells are present, resulting in lower levels of IFNγ and TNFα. CD8+ T-cell clones specific for DENV partially protect mice from lethal DENV challenge. T-regulatory cells may also function and expand in acute DENV infection.

Following primary infection by DENV, both serotype-specific and serotype cross-reactive memory T-cells are generated. Upon secondary exposure, both the protective and cross-reactive memory T-lymphocytes are activated, and the non-protective memory T cells can augment infection. Activated memory T cells recognize both conserved and altered peptide ligand epitopes. The antigen sequence differences depend on the specific DENV epitope and may affect the quality of the effector T-lymphocyte response. This in turn modifies the immunological repertoire and may be involved in the development of plasma leakage. A full agonist peptide will induce a full range of T cell responses, including the production of multiple cytokines (e.g. IFNγ, TNF, CCL4, etc.) and lysis of the infected cell. Because of sequence diversity between DENV serotypes, the memory T-cells (and B cells) that are re-activated during a secondary DENV infection may not have optimal avidity for epitopes of the new infecting virus. Thus, the ‘memory’ of the primary DENV infection alters the immune response to the secondary DENV infection, which can influence the clinical outcome. There is also a correlation between the level of T cell responses and disease severity. The phenomenon of low affinity for the current infecting serotype, but a high affinity for a past infection with a different serotype is referred to as ‘original antigenic sin,’ and is the net effect of an altered balance between a protective and pathological outcome. The pattern of antibody/T-cell responses in secondary DENV infections is also influenced by the sequence and interval between DENV. Regarding antibody dependent enhancement (ADE), memory T cell responses involving serotype cross-reactive, proliferative activity years after a primary infection could potentially alter the balance from a protective immune response toward a lesser or non-protective immune response. Of interest, most of the identified CD4+ and CD8+ T-cell epitopes reside in the NS3 protein, which represents only ˜20% of the DENV amino acid coding sequence.

An antigen capable of eliciting a natural antibody response to Dengue virus infection in humans may include the prM, E, or prM/E with proteins. Neutralizing antibodies are directed against the viral prM, E, or prM/E proteins and inhibit viral attachment, internalization, replication within cells, and egress from cells.

The DENV antigen (e.g., immunogen, SVP, VLP, etc.) sequences, immunogenic compositions, including, for example, vaccines, described herein induce a broadly reactive immune response (i.e., antibodies) that target and are directed against the proteins (e.g., prM, E, etc.) of multiple DENV subtypes, e.g., DENV1, DENV2, DENV3, DENV4, etc., or any fragments or any combinations thereof.

Broadly Reactive Dengue Virus Proteins, Subviral Particles (SVPs), and Virus-Like Particles (VLPs)

Provided, in some embodiments, are non-naturally occurring, broadly reactive, pan-epitopic Dengue virus immunogenic polypeptides (immunogens) and Dengue virus-like particles (VLPs), or subviral particles (SVPs), comprising a broadly reactive immunogen (e.g., DENV M polypeptide, DENV prM polypeptide, DENV envelope polypeptide DENV prM/E polypeptide, etc., or fragments or combinations thereof) containing diverse epitopes (antigenic determinants) that endow the immunogen with the ability to generate a broadly active immune response so as to treat dengue disease and its symptoms, either prophylactic or therapeutic, following administration and delivery to a susceptible subject. By way of example, representative Dengue virus envelope and prM/E immunogenic antigen sequences as described herein are presented in FIG. 1A, FIG. 1B, FIG. 9A, FIG. 9B, DENV1 (SEQ ID NOs:1, 2, 8), DENV2 (SEQ ID NOs:3, 9), DENV3 (SEQ ID NOs:4, 5, 10), DENV4 (SEQ ID NOs:6, 7, 11), any fragments thereof, or any combinations thereof as disclosed herein. In some embodiments, the broadly reactive, pan-epitopic immunogenic DENV envelope polypeptides are administered as SVPs or VLPs. In an embodiment, polynucleotide sequences encoding the DENV antigen sequences as described herein comprise an immunogen, immunogenic composition, subviral particle, virus-like particle, or vaccine.

It will be understood that Dengue virus antigen sequences and the immunogens described and provided herein are non-naturally occurring, broadly reactive and pan-epitopic, whether or not these characteristics and features are explicitly stated. It will also be appreciated that the Dengue virus antigen proteins, e.g., DENV membrane protein, DENV precursor membrane protein, DENV envelope protein, DENV prM/E protein, etc., or fragments or combinations thereof, etc., as described herein and used as immunogens are non-naturally occurring or synthetic antigens that elicit an immune response, e.g., neutralizing antibodies, in a subject.

The Dengue virus antigens described herein embrace seasonal or epidemic, pan-epitopic, broadly reactive antigens of Dengue virus and subtypes thereof, especially antigens containing sequences based on Dengue virus drift variants, where the antigens are designed to generate a broadly active immune response, particularly in the form of neutralizing antibodies, in a subject, particularly a human subject. Such antigens are beneficial as immunogens, which elicit an immune response (e.g., production of neutralizing antibodies) against the Dengue virus where multiple subtypes of Dengue co-circulate at one time.

The broadly reactive Dengue immunogenic antigens described here are derived from sequences of all subtypes of Dengue virus, and, as a consequence, stimulate a competent immune system to generate antibodies against multi-subtype antigenic epitopes (determinants) on the Dengue virus antigens, e.g., envelope polypeptides, following infection. Thus, the synthetic Dengue virus antigens comprise amino acid (or polynucleotide) sequences that will elicit greater numbers of neutralizing antibodies against all four Dengue virus subtypes (and against drift variants within and across multiple years of Dengue virus infection) compared with wild-type Dengue antigen sequences.

In one embodiment, non-naturally occurring (or synthetic), broadly reactive, immunogenic antigens (e.g., protein and glycoprotein antigens, immunogens, etc.) derived from Dengue virus (including different Dengue virus subtypes), that elicit a potent, broadly reactive immune response in a subject, particularly, a human subject. Such immunogenic antigens are also referred to as “immunogens” herein. Yet a further embodiment provides for a DENV antigen of the disclosure comprising an amino acid sequence that has 80% or greater (e.g., 85%, 90%, 95%, 98%, 99%, etc.) identity to an amino acid sequence of a Dengue virus antigen or antigenic fragment selected from SEQ ID Nos: 1-11 or any sequences set forth in FIG. 1A, FIG. 1B, FIG. 9A, FIG. 9B, or any fragments or combinations thereof.

Other embodiments of the disclosure are directed to immunogens that protect against disease caused by Dengue virus subtypes, spanning several years, including drifted strains not yet in existence. In an embodiment, fully synthetic Dengue protein antigens are featured. Such Dengue virus antigens are synthetic proteins not found in nature, yet they retain all of the functions of a natural Dengue viral protein and are immunogenic, i.e., they can elicit an immune response, in particular, a broadly reactive immune response in the form of neutralizing antibodies and/or reactive T-lymphocytes, following administration or delivery to, or introduction into, a subject. Also provided are immunogenic compositions (e.g., vaccines, medicinal products, etc.) comprising the synthetic Dengue virus protein antigens (e.g., immunogens, SVPs, VLPs, etc.) or nucleic acids encoding the antigens and the like.

In some embodiments a Dengue virus antigen amino acid sequence and a protein antigen having such a sequence, or any fragments or combinations thereof, may be used as an immunogen, or in an immunogenic composition, e.g., a vaccine, that elicits a broadly reactive immune response in a subject, particularly a human subject, to whom the composition, or vaccine, is administered. The Dengue virus immunogens comprise antigenic determinants that represent different “antigenic spaces” that are derived from the sequences of the four Dengue virus subtypes analyzed based on time periods (e.g., seasonal periods of time, either overlapping or non-overlapping time periods (e.g., calendar time periods, etc.), etc.) and/or geographical location (e.g., in wet, hot climate regions, rainforests, jungles, etc.). Such overlapping or non-overlapping time periods may encompass different intervals of time, for example, 5 months, 6 months, 7 months, eight months, nine months, 10 months, 11 months, 1 year, 2 years, 3 years, 4 years, 5 years, 6 years, 7 years, 8 years, 10 years, etc., or more, including time intervals therebetween.

In embodiments of the disclosure, the Dengue virus prM with the E protein (i.e., prM/E) form a Dengue virus antigen. In some embodiments, the Dengue virus prM/E of the disclosure include, for example, any of the sequences presented in FIG. 9A (SEQ ID NOs: 8-9) and FIG. 9B (SEQ ID NOs:10-11), or any fragments or combinations thereof, which elicit an immune response when administered in a subject. DENV1 (i.e., COBRA DV1) of SEQ ID NO:8 comprises a prM sequence from amino acid residues 1 to 167 at the N-terminal end and an adjacent E protein sequence from amino acid residues 168 to 662, where the prM portion is underlined in FIG. 9A. DENV2 (i.e., COBRA DV2) of SEQ ID NO:9 comprises a prM sequence from amino acid residues 1 to 167 at the N-terminal end and an adjacent E protein sequence from amino acid residues 168 to 662, where the prM portion is underlined in FIG. 9A. DENV3 (i.e., COBRA DV3) of SEQ ID NO:10 comprises a prM sequence from amino acid residues 1 to 167 at the N-terminal end and an adjacent E protein sequence from amino acid residues 168 to 660, where the prM portion is underlined in FIG. 9B. DENV4 (i.e., COBRA DV4) of SEQ ID NO:11 comprises a prM sequence from amino acid residues 1 to 166 at the N-terminal end and an adjacent E protein sequence from amino acid residues 167 to 661, where the prM portion is underlined in FIG. 9B.

Other embodiments may be directed to the Dengue virus E protein as a Dengue virus antigen. The Dengue virus antigen in some embodiments of the disclosure may include any of the sequences presented in FIG. 1A and FIG. 1B (SEQ ID NOs:1-7), or any fragments or combinations thereof, which elicit an immune response when administered in a subject. DENV1 of SEQ ID NO:1 and DENV1 American/1-495 of SEQ ID NO:2 each comprise a Dengue virus E protein of 495 amino acid residues. DENV2 of SEQ ID NO:3 comprises a Dengue virus E protein of 495 amino acid residues. DENV3 (1-493) of SEQ ID NO: 4 and DENV3 American/1-493 of SEQ ID NO:5 each comprise a Dengue virus E protein. DENV4 of SEQ ID NO:6 and DENV4 Asian/1-495 of SEQ ID NO:7 each comprise a Dengue virus E protein of 495 amino acid residues.

In a particular embodiment, the antigen sequences, compositions (e.g., vaccines, pharmaceutical compositions, immunogenic compositions, medicinal products, etc.), SVPs, and VLPs described herein for Dengue virus are advantageous as they generate an immune response (e.g., an antibody response, etc.) directed against more than one DENV serotype (e.g., 2 DENV serotypes, 3 DENV serotypes, 4 DENV serotypes, etc.) without enhancing disease. Such a property provides a highly useful medicinal product (e.g., immunogenic composition, vaccine, etc.) that protects against more than one DENV serotype, and in some instances, against all four serotypes of the Dengue virus, to avoid antibody dependent enhancement (ADE). ADE may occur when pre-existing antibodies to one DENV serotype do not neutralize, but instead enhance, a heterotypic infection by a Dengue virus subtype. As described herein, a DENV subviral particle (SVP) immunogenic composition (e.g., vaccine, medicinal product, etc.) which targets the envelope (E) glycoprotein, precursor Membrane (prM) glycoprotein, Membrane (M) glycoprotein, prM/E glycoprotein, and the like of Dengue virus, or fragments thereof, and which is broadly reactive against different DENV subtypes is provided. (See, Examples). As will be appreciated by one skilled in the art, a subviral particle (SVP) relates to or is a molecule (particle) comprising either genetic material or protein, and which is smaller than the intact Dengue virus particle, while maintaining features and properties of the virus. The broadly reactive and immunogenic Dengue virus antigen sequences that are capable of generating an immune response against Dengue virus subtypes, including all four Dengue virus serotypes (DENV1-4), as well as present and future Dengue virus strains, may be generated by a method such as described in co-pending provisional patent application No. 62/697,818, filed Jul. 13, 2018, the contents of which are incorporated herein by reference, which involves a consideration of parameters associated with Dengue virus, such as geography and time (e.g., a season, etc.) of infection, for example, amino acid sequences of Dengue virus subtypes and/or strains present in a geographical area or location, such as the Americas or Asia, during a selected period of time (e.g., a linear time range, etc.), in which the Dengue virus was isolated.

In an embodiment of the disclosure, the DENV SVPs or VLPs include a viral protein, (e.g., DENV M protein, DENV prM protein, DENV E protein, DENV prM/E protein, etc., fragments or combinations thereof). In some embodiments, the SVPs or VLPs may include other structural proteins of Dengue virus. The production of SVPs and VLPs has been described in the art and is within the skill and expertise of one of ordinary skill in the art. Briefly, and as described, DENV SVPs or VLPs can be produced by transfection of host cells with one or more plasmids containing polynucleotide sequences that encode a DENV protein (e.g., the membrane (M) protein, the precursor membrane (prM) protein, the envelope (E) protein, the prM/E protein, etc., or fragments or combinations thereof) of DENV. After incubation of the transfected cells for an appropriate time to allow for protein expression (such as, for example, approximately 72 hours, etc.), DENV SVPs or VLPs can be isolated from cell culture supernatants. DENV SVPs or VLPs can be purified from cell supernatants using procedures practiced in the art, for example, VLPs can isolated by low speed centrifugation (to remove cell debris), vacuum filtration and ultracentrifugation through 20% glycerol.

The DENV SVPs or VLPs can be used as immunogenic compositions or vaccines to elicit an immune response against Dengue viruses, subtypes and strains thereof. In particular, the component, broadly reactive, pan-epitopic DENV polypeptides of the immunogenic compositions or vaccines (or SVPs or VLPs or the like) contain antigenic (pan-epitopic) determinants that are broadly reactive and serve to elicit an immune response in a subject (e.g., the production of neutralizing antibodies and/or activated T-cells, etc.) that can treat a Dengue virus-infected subject (e.g., neutralize the infecting virus, etc.) and/or protect a subject against full-blown virus infection or the signs and symptoms thereof. In an embodiment, such immunogenic compositions or vaccines as described herein are effective in treating a secondary infection by a DENV subtype that is different from the DENV subtype which caused the primary infection. In an embodiment, such immunogenic compositions or vaccines as described herein provide highly useful medicinal products (e.g., immunogenic compositions, vaccines, pharmaceutical compositions, etc.) that protect against more than one serotype (e.g., all four serotypes, etc.) of the virus to avoid antibody dependent enhancement (ADE).

In an embodiment, the antigen sequence of a broadly reactive and immunogenic DENV antigen as described herein (e.g., DENV E protein antigen, DENV M protein antigen, DENV prM protein antigen, DENV prM/E protein antigen, etc., or fragments or combinations thereof) contains a diverse repertoire of epitopic determinants that can reflect antigenic drift and sequence variability in the DENV's antigenic proteins, for example, over time periods (e.g., seasons, outbreaks, epidemics, etc.), or in different geographic locations. In particular, a DENV E protein antigen, a DENV M protein antigen, a DENV prM protein antigen, a DENV prM/E protein antigen, etc., or fragments or combinations thereof, as described herein (see, e.g., FIG. 1A, FIG. 1B, FIG. 9A, FIG. 9B, SEQ ID NOs:1-11, etc.) can comprise an amino acid sequence that contains antigenic determinants (or epitopes) derived from sequence diverse DENV subtypes and strains, including drift variants, against which broadly reactive neutralizing antibodies can be raised, especially when the antigen is used as an immunogenic product, (e.g., an immunogen, etc.), e.g., an antiviral vaccine, immunogenic composition, medicinal product, etc.) that is introduced into a subject.

In an aspect, the DENV antigen amino acid sequences provide a composite, immunogenic antigen sequence, which includes epitopic determinants ultimately derivable from both past and more recent DENV causing infection or disease, and/or from viruses in different geographical locales, and/or from different subtypes of DENV (DENV1-4), i.e., a “pan-epitopic” antigen that elicits a broadly reactive immune response when used as an immunogen, a vaccine, SVP, or VLP. In an embodiment, the immunogenic DENV E protein antigen, a DENV M, protein antigen, a DENV prM protein antigen, a DENV prM/E protein antigen, etc., or fragments or combinations thereof, as described herein (see, e.g., FIG. 1A, FIG. 1B, FIG. 9A, FIG. 9B, SEQ ID NOs:1-11, etc.) sequences encompass epitopes that generate antibodies that are directed against the protein of more than one, or all, of the serotypes/subtypes of DENV. In an embodiment, the immunogenic DENV E protein antigen, a DENV M, protein antigen, a DENV prM protein antigen, a DENV prM/E protein antigen, etc., or fragments or combinations thereof, as described herein (see, e.g., FIG. 1A, FIG. 1B, FIG. 9A, FIG. 9B, SEQ ID NOs:1-11, etc.) sequences encompass epitopes that result from antigenic changes in the sequences of DENV E protein surface antigens, a DENV M, protein antigen, a DENV prM protein antigens, a DENV prM/E protein antigens, etc., or fragments or combinations thereof, as described herein (see, e.g., FIG. 1A, FIG. 1B, FIG. 9A, FIG. 9B, SEQ ID NOs:1-11, etc.) that arise from point mutations during viral replication, giving rise to new DENV variants. As a result, the administration to a subject of an DENV immunogen as described herein can elicit a broadly reactive immune response in the subject that is directed against epitopes reflecting such antigenic changes.

Because the broadly reactive DENV antigens and the sequences thereof as described herein and used as an immunogen or immunogenic composition, such as a vaccine, elicit a broadly reactive immune response in an immunocompetent subject, they provide a superior vaccine that captures the antigenic epitopes of many different DENV isolates (subtypes or strains), against which broadly active immune responses (e.g., broadly active neutralizing antibodies) are generated. It is noted that the terms “broadly active” and “broadly reactive” are used synonymously herein.

In an embodiment, the Dengue virus antigen as described herein is a polypeptide or peptide antigen of Dengue virus which currently causes disease or infection and its symptoms, such as Dengue disease in its various forms, and/or which is native to certain geographical locales. In another embodiment, the Dengue virus antigen is a polypeptide or peptide antigen which will, in future, cause disease and symptoms of Dengue virus infection. In an embodiment, the Dengue antigen is a polynucleotide sequence. In an embodiment, the Dengue virus antigen is a polynucleotide sequence that encodes a polypeptide or peptide antigen as described herein. By way of example, representative broadly reactive DENV immunogens are shown in FIG. 1A, FIG. 1B, FIG. 9A, FIG. 9B, SEQ ID NOs:1-11, etc., or fragments or combinations thereof.

In another embodiment, the DENV immunogen sequence described herein is expressed in a cell as a polypeptide, protein, or peptide. In an embodiment, the DENV immunogen is isolated and/or purified. In an embodiment, the immunogen is formulated for administration to a subject in need. In an embodiment, the immunogen is administered to a subject in need thereof in an effective amount to elicit an immune response in the subject. In an embodiment, the immune response elicits neutralizing antibodies. In an embodiment, the immune response is prophylactic or therapeutic.

In an embodiment, a non-naturally occurring DENV immunogen (or immunogen sequence) (e.g., a vaccine, medicinal product, or immunogenic composition, etc.) is provided that elicits a broadly reactive immune response in a subject following introduction, administration, or delivery of the immunogen to the subject. The route of introduction, administration, or delivery is not limited and may include, for example, intravenous, subcutaneous, intramuscular, oral, etc. routes. The vaccine may be therapeutic (e.g., administered to a subject following a symptom of disease caused by DENV or prophylactic (protective), (e.g., administered to a subject prior to the subject having or expressing a symptom of disease, or full-blown disease, caused by DENV, etc.).

In an embodiment, the final amino acid sequence of the antigen (e.g., a DENV E protein antigen, a DENV M protein antigen, a DENV prM protein antigen, a DENV prM/E protein antigen, etc., or fragments or combinations thereof, as described herein (see, e.g., FIG. 1A, FIG. 1B, FIG. 9A, FIG. 9B, SEQ ID NOs:1-11, etc.)) is reverse translated and optimized for expression in mammalian cells. As will be appreciated by the skilled practitioner in the art, optimization of the nucleic acid sequence includes optimization of the codons for expression of a sequence in mammalian cells and RNA optimization (such as, e.g., RNA stability).

In an embodiment, an isolated nucleic acid molecule (polynucleotide) comprising a nucleotide sequence encoding a polypeptide or peptide antigen, such as a DENV polypeptide (e.g., a DENV E polypeptide, DENV M protein, DENV prM protein, a DENV prM/E protein, etc., or fragments or combinations thereof, as described herein, (see, e.g., FIG. 1A, FIG. 1B, FIG. 9A, FIG. 9B, SEQ ID NOs:1-11, etc.)), is provided. In certain embodiments, the nucleotide sequence encoding the DENV polypeptide is at least 94%, at least 95%, at least 96%, at least 97%, at least 98% or at least 99% identical to a polynucleotide encoding a DENV polypeptide (e.g., DENV E polypeptide, a DENV M polypeptide, a DENV prM polypeptide, a DENV prM/E polypeptide, etc., or fragments or combinations thereof, as described herein) sequence shown in FIG. 1A, FIG. 1B, FIG. 9A, FIG. 9B, SEQ ID NOs:1-11, etc.

In other embodiments, the nucleotide sequence encoding the DENV polypeptide (e.g., DENV E polypeptide, a DENV M polypeptide, a DENV prM polypeptide, a DENV prM/E polypeptide, etc., or fragments or combinations thereof, as described herein), that is at least 90%, at least 91%, at least 92%, at least 93%, at least 94%, at least 95%, at least 96%, at least 97%, at least 98%, or at least 99% identical to a polynucleotide encoding DENV polypeptide (e.g., DENV E polypeptide, a DENV M polypeptide, a DENV prM polypeptide, a DENV prM/E polypeptide, etc., or fragments or combinations thereof, as described herein) sequence shown in FIG. 1A, FIG. 1B, FIG. 9A, FIG. 9B, SEQ ID NOs:1-11, etc., where the sequence may lack the start codon encoding an N-terminal methionine.

Vectors containing a nucleotide sequence encoding a non-naturally occurring, broadly reactive polypeptide or peptide antigen, such as DENV polypeptide (e.g., DENV E polypeptide, a DENV M polypeptide, a DENV prM polypeptide, a DENV prM/E polypeptide, etc., or fragments or combinations thereof, as described herein) are provided. In some embodiments, the vectors comprise a nucleotide sequence encoding the polypeptide or peptide antigen, such as a DENV polypeptide antigen (e.g., a DENV E polypeptide antigen, a DENV M polypeptide antigen, a DENV prM polypeptide antigen, a DENV prM/E polypeptide antigen, etc., or fragments or combinations thereof, as described herein), that is at least 94%, at least 95%, at least 96%, at least 97%, at least 98% or at least 99% identical to a polynucleotide encoding a DENV polypeptide (e.g., a DENV E polypeptide, a DENV M polypeptide, a DENV prM polypeptide, a DENV prM/E polypeptide, etc., or fragments or combinations thereof, as described herein) sequence shown in FIG. 1A, FIG. 1B, FIG. 9A, FIG. 9B, SEQ ID NOs:1-11, etc. In some embodiments, the vector further includes a promoter operably linked to the nucleotide sequence encoding the DENV polypeptide (e.g., a DENV E polypeptide, a DENV M polypeptide, a DENV prM polypeptide, a DENV prM/E polypeptide, etc., or fragments or combinations thereof, as described herein). In a particular embodiment, the promoter is a cytomegalovirus (CMV) promoter. In some embodiments, the nucleotide sequence of the vector is at least 85%, at least 90%, at least 95%, at least 98% or at least 99% identical to a polynucleotide encoding a DENV polypeptide (e.g., DENV E polypeptide, a DENV M polypeptide, a DENV prM polypeptide, a DENV prM/E polypeptide, etc., or fragments or combinations thereof, as described herein) sequence shown in FIG. 1A, FIG. 1B, FIG. 9A, FIG. 9B, SEQ ID NOs:1-11, etc. In particular embodiments, the nucleotide sequence of the vector comprises the polynucleotide encoding a DENV polypeptide (e.g., a DENV E polypeptide, a DENV M polypeptide, a DENV prM polypeptide, a DENV prM/E polypeptide, etc., or fragments or combinations thereof, as described herein) sequence shown in FIG. 1A, FIG. 1B, FIG. 9A, FIG. 9B, SEQ ID NOs:1-11, etc. In embodiments, the vector is a prokaryotic or eukaryotic vector. In an embodiment, the vector is an expression vector, such as a eukaryotic (e.g., mammalian, etc.) expression vector. In another embodiment, the vector is a plasmid (prokaryotic or bacterial) vector. In another embodiment, the vector is a viral vector. The vectors used to express a Dengue virus antigen (e.g., a DENV protein, DENV M protein, DENV prM protein, DENY E protein, DENV prM/E protein, etc., fragments or combinations thereof), as described herein may be any suitable expression vectors known and used in the art. The vectors can be, for example, mammalian expression vectors or viral vectors. In some embodiments, the vector is the pTR600 expression vector (U.S. Patent Application Publication No. 2002/0106798, herein incorporated by reference; Ross et al., 2000, Nat Immunol. 1(2):102-103; and Green et al., 2001, Vaccine 20:242-248).

Provided are Dengue virus-derived, non-naturally occurring polypeptide antigens (e.g., DENV polypeptide antigens, DENV E polypeptide antigens, DENV M polypeptide antigens, DENV prM polypeptide antigens, DENV prM/E polypeptide antigens, etc., fragments or combinations thereof) produced by transfecting a host cell with an expression vector as known and used in the art under conditions sufficient to allow for expression of the polypeptide (e.g., a DENV polypeptide, a DENV E polypeptide, DENV M polypeptide, DENV prM polypeptide, DENV prM/E polypeptide, etc., fragments or combinations thereof) in the cell. Isolated cells containing the vectors are also provided.

Also provided are non-naturally occurring, broadly reactive, pan-epitopic DENV antigen polypeptides as described herein, such as but not limited to, pan-epitopic, broadly reactive DENV E polypeptides, DENV M polypeptides, DENV prM polypeptides, DENV prM/E polypeptides, etc., fragments or combinations thereof. In certain embodiments, the amino acid sequence of the polypeptide is at least 95% to 99% (inclusive) identical to the amino acid sequence of an envelope polypeptide of DENV, as shown in FIG. 1A, FIG. 1B, SEQ ID NOs:1-7, fragments or combinations thereof, etc. In particular embodiments, the amino acid sequence of the DENV E polypeptide that is at least 95% to 99% (inclusive) identical to the amino acid sequence of an E polypeptide shown in FIGS. 1A and 1B, lacks the N-terminal methionine residue. In another embodiment, the amino acid sequence of the Dengue polypeptide is at least 95% to 99% (inclusive) identical to amino acids (e.g., 1-493, 1-495, 1-496, etc., or immunogenic fragments or combinations thereof) of the DENV polypeptides shown in FIGS. 1A and 1B (SEQ ID NOs:1-7).

In other embodiments, the amino acid sequence of the DENV polypeptide is at least 95% to 99% (inclusive) identical to the amino acid sequence of the precursor membrane (prM)/Envelope (E) fusion proteins of DENV, as shown in FIGS. 9A and 9B (SEQ ID NOs:8-11). In further embodiments, the amino acid sequence of the DENV E polypeptide portion of the prM/E fusion protein that is at least 95% to 99% (inclusive) identical to the amino acid sequence of an E polypeptide shown in FIGS. 9A and 9B, lacks the N-terminal methionine residue. In yet another embodiment, the amino acid sequence of the Dengue virus prM/E polypeptide is at least 95% to 99% (inclusive) identical to amino acids of the prM portion or to amino acids of the E portion of the DENV polypeptides shown in FIGS. 9A and 9B, where the prM portion at the N-terminal end is underlined and the E portion at the C-terminal end is without any underlined text.

In some embodiments, fusion proteins comprising the broadly reactive, pan-epitopic DENV antigen polypeptides described herein, e.g., without limitation, the DENV envelope polypeptides, DENV prM polypeptides, DENV prM/E polypeptides, etc., or fragments or combinations thereof, are also provided. In some embodiments, the DENV envelope polypeptide can be fused to any heterologous amino acid sequence to form a fusion protein. By way of example, peptide components of DENV polypeptides may be generated independently and then fused together to produce an intact DENV polypeptide antigen (e.g., comprising 463, 465, 493, 495, 496, 660, 661, 662, etc. amino acids), for use as an immunogen. For example, sequences for the DENV prM polypeptide may be fused to that of the DENV E polypeptide to form DENV prM/E polypeptides for use as an immunogen, where the sequences may include those presented in FIGS. 9A and 9B or SEQ ID NOs: 8-11, fragments or combinations thereof, etc.

Also provided are subviral particles (SVPs) or virus-like particles (VLPs), in particular, DENV SVPs or VLPs, containing a pan-epitopic, broadly reactive protein antigen (e.g., DENV polypeptide antigens, DENV M protein antigens, DENV prM protein antigens, DENV envelope (E) protein antigens, DENV prM/E protein antigens, etc., or fragments or combinations thereof, etc.) as described herein. In certain embodiments, the E protein of the VLP is at least or equal to 90% (e.g., 94%, 95%, 96%, 97%, 98%, 99%, 100%, etc.) identical to the DENV E proteins as shown in FIGS. 1A and 1B. In other embodiments, the prM/E fusion protein of the VLP is at least or equal to 90% (e.g., 94%, 95%, 96%, 97%, 98%, 99%, 100%, etc.) identical to the DENV prM/E fusion proteins as shown in FIGS. 9A and 9B. The DENV SVPs or VLPs can further include any additional viral proteins necessary to form the virus particle. In certain embodiments, the virus or Dengue VLPs or SVPs further include another Dengue virus protein.

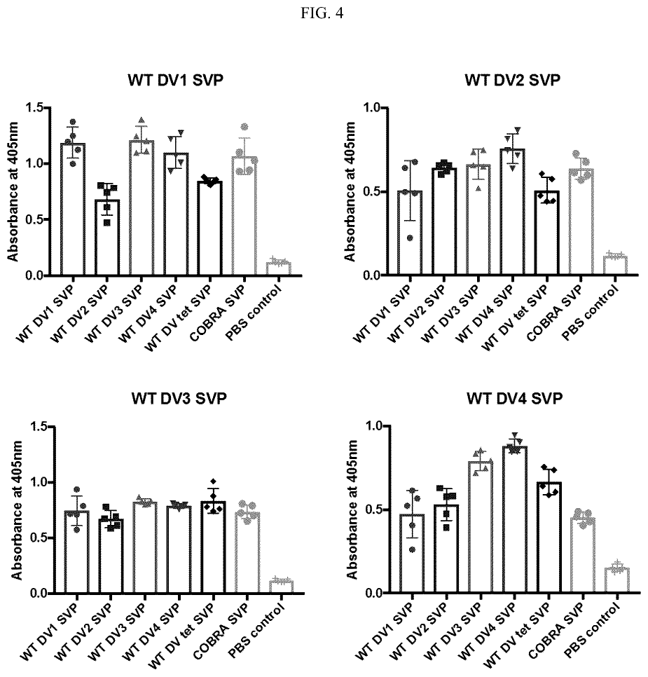

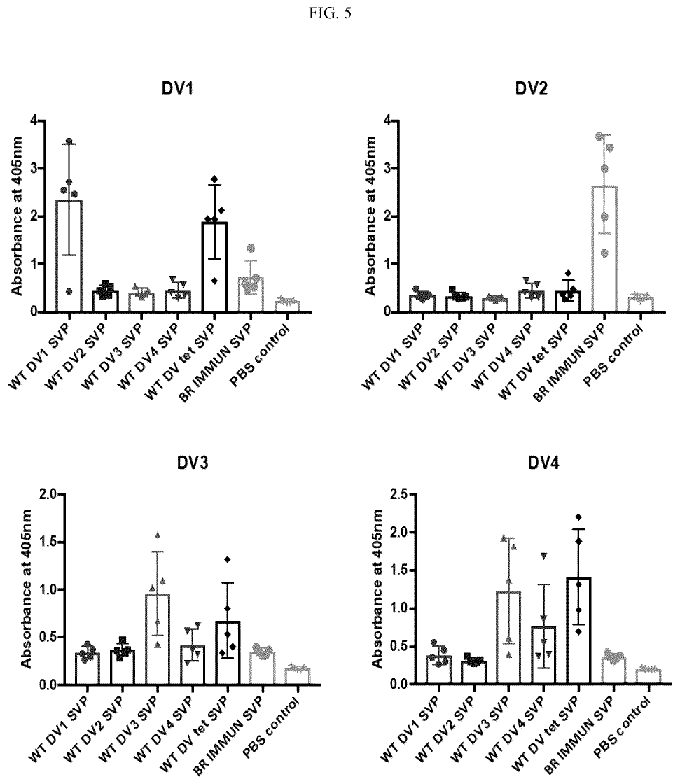

Yet other embodiments provide a DENV SVP or VLP containing a DENV polypeptide (e.g., a DENV envelope polypeptide, DENV M polypeptide, DENV prM polypeptide, DENV prM/E polypeptide, etc., fragments or combinations thereof), as described herein, produced by transfecting a host cell with a vector containing a polynucleotide encoding the DENV polypeptide. Also provided in an embodiment is a DENV SVP or VLP containing a DENV polypeptide (e.g., DENV E polypeptide, DENV M polypeptide, DENV prM polypeptide, DENV prM/E polypeptide, etc., fragments or combinations thereof), as described herein, produced by transfecting a host cell with a vector encoding the DENV polypeptide (e.g., DENV E polypeptide, DENV M polypeptide, DENV prM polypeptide, DENV prM/E polypeptide, etc., fragments or combinations thereof), under conditions sufficient to allow for expression of the DENV proteins. Such SVPs or VLPs comprising the sequences as presented in, for example, FIG. 1A, FIG. 1B, FIG. 9A, FIG. 9B, SEQ ID NOs:1-11, etc. and used as immunogens generate antibodies having high neutralization titers against the four subtypes of DENV and strains thereof, as observed in, for example, FIGS. 4, 5, 8A-8C, 11A-11D, 12A-12F, 13, 14, Examples 5-7, etc.

Collections of plasmids (vectors) are also contemplated. In certain embodiments, the collection of plasmids includes plasmid encoding a broadly reactive DENV protein (e.g., DENV envelope protein, DENV M protein, DENV prM protein, DENV prM/E protein, etc., fragments or combinations thereof) as described herein, as well as plasmids encoding other DENV proteins, such as, for example, a structural protein other than an envelope protein, or another envelope protein, etc. In some embodiments, the nucleotide sequence encoding an envelope protein of the DENV envelope-encoding plasmid is at least 90% (e.g., 94%, 95%, 96%, 97%, 98%, 99%, 100%, etc.) identical to a polynucleotide encoding a DENV envelope polypeptide amino acid sequence or a DENV prM/E polypeptide amino acid sequence as shown in FIG. 1A, FIG. 1B, FIG. 9A, FIG. 9B, SEQ ID NOs:1-11, etc. In some embodiments, the nucleotide sequence encoding a codon-optimized DENV protein of the DENV envelope-encoding plasmid is at least 90% (e.g., 94%, 95%, 96%, 97%, 98%, 99%, 100%, etc.) identical to a polynucleotide encoding a DENV envelope polypeptide amino acid sequence or a DENV prM/E polypeptide amino acid sequence as shown in FIG. 1A, FIG. 1B, FIG. 9A, FIG. 9B, SEQ ID NOs:1-11, etc.

In the context of the present disclosure, “broadly reactive” or “broadly active” means that the DENV protein (e.g., a DENV envelope protein sequence) is immunogenic and contains a diversity of epitopes (antigenic determinants; pan-epitopic) that elicit in a subject an immune response (e.g., neutralizing antibodies directed against the diversity of DENV protein (e.g., envelope protein, membrane protein, prM protein, prM/E protein, etc.), epitopes, frequently accompanied by a T-cell response, etc.) sufficient to treat Dengue virus disease or infection, and/or to inhibit, neutralize, or prevent infection, caused by most or all Dengue viruses within a specific subtype, or by other DENV subtypes. In embodiments, the broadly reactive, DENV-derived antigen protein (e.g., DENV envelope protein, DENV M protein, DENV prM protein, DENV prM/E protein, etc., fragments or combinations thereof), elicits a protective immune response against most or all known Dengue virus subtypes, such as 80% (e.g., 85%, 90%, 95%, 96%-99%, etc.) of the known Dengue virus subtypes or isolates thereof

Compositions and Pharmaceutical Compositions for Administration

A Dengue immunogenic protein, or immunogen, as described herein can be employed in a composition (e.g., vaccine, immunogenic composition, pharmaceutical composition, medicinal product, etc.) that may afford protection against many Dengue virus strains over several years. The broadly reactive Dengue virus immunogens and vaccines described herein are advantageous in that they are designed to provide broader and longer-lasting protection against several Dengue strains (or subtypes) prevalent in different geographical locations. Provided by the immunogens and their sequences as described herein is a universal and broad-spectrum Dengue virus vaccine that may alleviate the need for the administration of different vaccines (immunogenic compositions) against subtypes of Dengue virus that require repeated administration.

The immunogenic Dengue virus antigens described herein may be used in immunogenic compositions (e.g., Dengue vaccines, pharmaceutical compositions, medicinal products, etc.) that afford protective immunity against infection and disease caused by one or more Dengue virus serotypes (subtypes) in a subject. The protective immunity is provided in the subject through the elicitation of potent, broadly reactive, anti-Dengue virus protein specific antibody responses that protect the subject against the four Dengue virus serotypes, drifted, Dengue virus strains and pandemic Dengue virus strains. The immunogenic compositions and vaccines provide an advantage over prior and traditional immunogenic compositions and vaccines directed against Dengue virus, which typically depend on the selection of candidate vaccine viruses by public health authorities following analysis of data collected through active surveillance of Dengue viruses that periodically or episodically circulate in certain time periods.

Compositions comprising a broadly reactive, pan-epitopic DENV protein, (e.g., E protein, M protein, prM protein, prM/E protein, etc., or fragments or combinations thereof) or a fusion protein or VLP comprising such a broadly reactive DENV protein as described herein are provided. In some embodiments, the compositions further comprise a pharmaceutically acceptable carrier, excipient, or vehicle. In other embodiments, an adjuvant (e.g., a pharmacological agent, immunological agent, etc., where the agent modifies or boosts an immune response, e.g., to produce more antibodies that are longer-lasting, etc.) is also employed. For example, without limitation, the adjuvant can be an inorganic compound, such as alum, aluminum hydroxide, or aluminum phosphate; mineral or paraffin oil; squalene; detergents such as Quil A; plant saponins; Freund's complete or incomplete adjuvant, a biological adjuvant (e.g., cytokines (such as IL-1, IL-2, IL-12, etc.), etc.); bacterial products such as killed Bordetella pertussis, toxoids; or immunostimulatory oligonucleotides (such as CpG oligonucleotides), etc.).

Compositions and preparations (e.g., physiologically or pharmaceutically acceptable compositions) containing the non-naturally occurring, broadly reactive, pan-epitopic DENV polypeptides (e.g., DENV membrane polypeptide, DENV precursor membrane polypeptide, DENV envelope polypeptide, DENV prM/E protein, etc., or fragments or combinations thereof, etc.) and Dengue subviral particles or virus-like particles (VLPs) for parenteral administration include, without limitation, sterile aqueous or non-aqueous solutions, suspensions, emulsions, and the like. Nonlimiting examples of non-aqueous solvents include propylene glycol, polyethylene glycol, vegetable oils, such as olive oil and canola oil, injectable organic esters (such as ethyl oleate), etc. Aqueous carriers include water, alcoholic/aqueous solutions, emulsions or suspensions, including saline and buffered media. Parenteral vehicles include, for example, sodium chloride solution, Ringer's dextrose, dextrose and sodium chloride, lactated Ringer's, fixed oils, etc. Intravenous vehicles include, for example, fluid and nutrient replenishers, electrolyte replenishers (such as those based on Ringer's dextrose), and the like. Preservatives and other additives may also be present in such compositions and preparations, such as, for example, antimicrobials, antioxidants, chelating agents, colorants, stabilizers, inert gases, and the like.

Some of the compositions may be administered as a pharmaceutically acceptable acid- or base-addition salt, formed by reaction with inorganic acids, such as but not limited to, hydrochloric acid, hydrobromic acid, perchloric acid, nitric acid, thiocyanic acid, sulfuric acid, phosphoric acid, organic acids, such as but not limited to, formic acid, acetic acid, propionic acid, glycolic acid, lactic acid, pyruvic acid, oxalic acid, malonic acid, succinic acid, maleic acid, fumaric acid, or by reaction with an inorganic base such as but not limited to, sodium hydroxide, ammonium hydroxide, potassium hydroxide, and organic bases such as but not limited to, mono-, di-, tri-alkyl and aryl amines, substituted ethanolamines, etc., or any combinations thereof.

Provided herein are pharmaceutical compositions which include a therapeutically effective amount of a non-naturally occurring, broadly reactive, pan-epitopic, Dengue virus protein antigen, or Dengue virus SVPs or VLPs, alone, or in combination with a pharmaceutically acceptable carrier. Pharmaceutically acceptable carriers include, but are not limited to, saline, buffered saline, dextrose, water, glycerol, ethanol, etc., and combinations thereof. The carrier and composition can be sterile, and the formulation suits the mode of administration. Non-limiting formulations comprising a non-naturally occurring, broadly reactive, pan-epitopic, Dengue virus protein antigen, DENV SVPs, DENV VLPs, etc. may be in the form of: a nasal, oral or injectable liquid suspension Or solution, or in solid or semi-solid form, powders, pellets, capsules, granules, sugar-coated pills, gelules, sprays, cachets, pills, tablets, pastes, implants, gels, etc. The composition can also contain minor amounts of wetting or emulsifying agents, or pH buffering agents. The composition can be a liquid or aqueous solution, suspension, emulsion, dispersion, tablet, pill, capsule, powder, sustained release formulation, etc. A liquid or aqueous composition can be lyophilized and reconstituted with a solution or buffer prior to use. The composition can be formulated as a suppository, with traditional binders and carriers such as triglycerides. Oral formulations can include standard carriers, such as but not limited to, pharmaceutical grades of mannitol, lactose, starch, magnesium stearate, sodium saccharine, cellulose, magnesium carbonate, etc. Any of the commonly known pharmaceutical carriers, such as but not limited to, sterile saline solution or sesame oil, can be used. The medium can also contain conventional pharmaceutical adjunct materials such as, for example, pharmaceutically acceptable salts to adjust the osmotic pressure, buffers, preservatives, and the like. Other media that can be used in the compositions and administration methods as described are normal saline and sesame oil.

Another embodiment may be directed to a pharmaceutical composition that may be formulated as a veterinary composition comprising a non-naturally occurring, broadly reactive, pan-epitopic, Dengue virus protein antigen, DENV SVPs, DENV VLPs, etc., intended for use with subjects other than humans. The veterinary compositions according to the present disclosure may be in any appropriate form to suit the requested administration modes, for instance, nasal, oral, intradermic, cutaneous, parenteral, etc. In one embodiment, the veterinary composition is in a form intended for oral administration, for instance, when the domestic animal eats, the veterinary composition may either be mixed in the food ration or directly administered into the mouth. The veterinary compositions of the disclosure may be in the form of a nasal, oral or injectable liquid suspension or solution, or in solid or semi-solid form, powders, pellets, capsules, granules, sugar-coated pills, gelules, sprays, cachets, pills, tablets, pastes, implants, gels, etc. In a particular embodiment, the compositions are in the form of an oral solid form, such as but not limited to, tablets. In some embodiments, the veterinary compositions may have an effective amount of the Dengue virus antigen, immunogen, vaccine composition, etc. for a specific species of animal (e.g., lamb, goat, bovine, equine, canine, feline, avian, such as chicken or turkey, etc.). In certain embodiments, the veterinary composition is formulated for the treatment or prophylaxis of disease transmitted by mosquitoes or ticks carrying Dengue virus. In certain implementations the veterinary formulations may be useful for the treatment or prophylaxis of disease in, for example, lambs, goats, bovines, equines, canines, felines, avians, koalas, rats, sheep, fish, amphibians (e.g., frogs), reptiles, etc.

Methods of Treatment or Prevention, Administration, and Delivery

Methods of treating a disease or infection, or symptoms thereof, caused by Dengue virus are provided. The methods comprise administering a therapeutically effective amount of a broadly reactive, pan-epitopic immunogen (e.g., antigen, SVP, VLP, immunogenic composition, vaccine, medicinal product, etc.) as described herein, or a composition comprising the immunogen (e.g., pharmaceutical composition, or a vaccine, medicinal product, SVP, VLP, SVP or VLP vaccine, etc.) as described herein, to a subject (e.g., a mammal, human, etc.) in order to treat or protect against Dengue virus. One embodiment involves a method of treating a subject suffering from, or at risk of or susceptible to disease or infection, or a symptom thereof, caused by Dengue virus. The method includes administering to the subject (e.g., a mammalian subject, a human subject, etc.), an amount (e.g., an effective amount, a therapeutic amount, etc.) of a Dengue virus immunogenic composition, such as but not limited to, a vaccine, a medicinal product, etc., comprising a non-naturally occurring, broadly reactive, pan-epitopic, Dengue virus antigen polypeptide (e.g., DENV envelope (E) polypeptide (e.g., SEQ ID NOs:1-7, etc.), DENV M polypeptide, DENV prM polypeptide, DENV prM/E polypeptide (e.g., SEQ ID NOs:8-11, etc.), etc.), DENV polypeptide SVPs, DENV polypeptide VLPs, etc., fragments or combinations thereof), sufficient to treat the disease, infection, or symptoms thereof, caused by Dengue virus under conditions in which the disease, infection, and/or the symptoms thereof are treated. A further embodiment provides for the use of an effective amount of a Dengue virus immunogenic composition (e.g., vaccine, medicinal product, etc.) comprising a non-naturally occurring, broadly reactive, pan-epitopic, Dengue virus antigen polypeptide (e.g., DENV envelope (E) polypeptide (e.g., SEQ ID NOs:1-7, etc.), DENV M polypeptide, DENV prM polypeptide, DENV prM/E polypeptide (e.g., SEQ ID NOs:8-11, etc.), etc.), DENV polypeptide SVPs, DENV polypeptide VLPs, etc., fragments or combinations thereof) for the treatment, prevention, and/or attenuation of symptoms of Dengue virus, wherein the use produces an immune response.

In another embodiment, the methods described herein include administering to the subject (including a human subject identified as in need of such treatment) an effective amount of a non-naturally occurring, broadly reactive, pan-epitopic, Dengue virus antigen polypeptide, such as but not limited to, DENV E polypeptide (e.g., SEQ ID NOs:1-7, etc.), DENV M polypeptide, DENV prM polypeptide, DENV prM/E polypeptide (E.g., SEQ ID NOs:8-11, etc.), etc., or administering an effective amount of a composition (e.g., vaccine, medicinal product, therapeutic composition, etc.) comprising a Dengue virus antigen or immunogen as described herein to produce an immune response. The methods of treatment, prevention, and/or attenuation of Dengue virus symptoms are suitably administered to subjects, particularly humans, suffering from, having, susceptible to, or at risk of having a dengue virus disease, disorder, infection, or symptom thereof, namely, dengue fever (DF), characterized by high fever, headache, stomachache, rash, myalgia, and arthralgia, or severe dengue (also known as Dengue Hemorrhagic Fever (DHF)), and Dengue Shock Syndrome (DSS). Identifying a subject in need of such treatment can be based on the judgment of the subject or of a health care professional and can be subjective (e.g. opinion, etc.) or objective (e.g. measurable by a test or diagnostic method, etc.). Briefly, the determination of those subjects who are in need of treatment or who are “at risk” or “susceptible” can be made by any objective or subjective determination by a diagnostic test (e.g., genetic test, enzyme, protein marker assay, etc.), marker analysis, family history, and the like, including an opinion of the subject or a health care provider. The non-naturally occurring, broadly reactive, pan-epitopic DENV immunogens, such as DENV envelope polypeptide immunogens and vaccines as described herein, may also be used in the treatment of any other disorders in which infection or disease caused by Dengue virus may be implicated. A subject undergoing treatment can be a non-human mammal, such as a veterinary subject, or a human subject (also referred to in some embodiments as a “patient”).

In addition, prophylactic methods of preventing or protecting against a disease or infection, or symptoms thereof, caused by Dengue virus are provided. Such methods comprise administering a therapeutically effective amount of a pharmaceutical composition comprising a Dengue immunogenic composition or vaccine (e.g., a Dengue SVP or VLP vaccine, etc.) as described herein to a subject (e.g., a mammal such as a human, etc.), in particular, prior to infection of the subject or prior to onset of the disease, such as DENV-associated disease. Another embodiment provides for an effective amount of a Dengue virus immunogenic composition or pharmaceutical composition (e.g., vaccine, DENV SVP vaccine, DENV VLP vaccine, medicinal product, etc.) comprising a non-naturally occurring, broadly reactive, pan-epitopic, Dengue virus antigen or immunogen (e.g., DENV envelope (E) polypeptide (e.g., SEQ ID NOs:1-7, etc.), DENV M polypeptide, DENV prM polypeptide, DENV prM/E polypeptide (e.g., SEQ ID NOs:8-11, etc.), etc.), DENV polypeptide SVPs, DENV polypeptide VLPs, etc., fragments or combinations thereof) for use in the treatment, prevention, protection, or attenuation of Dengue virus and/or its symptoms or for the treatment, prevention, protection, or attenuation of Dengue virus and/or its symptoms.

A further embodiment is directed to a use of a Dengue virus substance or composition (e.g., Dengue virus immunogenic composition, Dengue virus vaccine, Dengue virus SVP, Dengue virus VLP, medicinal product, etc.) comprising a non-naturally occurring, broadly reactive, pan-epitopic, Dengue virus antigen or immunogen (e.g., DENV envelope (E) polypeptide (e.g., SEQ ID NOs:1-7, etc.), DENV M polypeptide, DENV prM polypeptide, DENV prM/E polypeptide (e.g., SEQ ID NOs:8-11, etc.), etc.), DENV polypeptide SVPs, DENV polypeptide VLPs, etc., fragments or combinations thereof) for the manufacture of a medicament for a therapeutic, prophylactic, or preventative application of Dengue virus and/or its symptoms. Yet another embodiment provides for a method of preparing a Dengue virus substance or composition (e.g., Dengue virus immunogenic composition, Dengue virus vaccine, Dengue virus SVP, Dengue virus VLP, medicinal product, etc.) comprising a non-naturally occurring, broadly reactive, pan-epitopic, Dengue virus antigen or immunogen (e.g., DENV envelope (E) polypeptide (e.g., SEQ ID NOs:1-7, etc.), DENV M polypeptide, DENV prM polypeptide, DENV prM/E polypeptide (e.g., SEQ ID NOs:8-11, etc.), etc.), DENV polypeptide SVPs, DENV polypeptide VLPs, etc., fragments or combinations thereof) for the manufacture of a medicament for a therapeutic, prophylactic, or preventative application of Dengue virus and/or its symptoms or for the treatment, prevention, protection, or attenuation of Dengue virus and/or its symptoms.

In other embodiments, a method of monitoring the progress of an Dengue virus infection or disease caused by Dengue virus, or monitoring treatment of the Dengue infection or disease is provided. The method includes determining a level of a diagnostic marker or biomarker (e.g., a Dengue virus protein, DENV envelope protein, DENV M protein, DENV prM protein, DENV prM/E protein, etc.), or a diagnostic measurement (e.g., screening assay, detection assay, monitoring assay, etc.) in a subject suffering from or susceptible to infection, disease or symptoms thereof associated with Dengue virus, in which the subject has been administered an amount (e.g., an effective amount, a therapeutic amount, etc.) of a non-naturally occurring, broadly reactive, pan-epitopic Dengue virus protein (e.g., DENV M protein, DENV prM protein, DENV envelope (E) protein, DENV prM/E protein, etc., fragments or combinations thereof, etc.) as described herein, or an immunogenic composition or vaccine as described herein, sufficient to treat the infection, disease, or symptoms thereof. The level or amount of the marker or biomarker (e.g., protein, etc.) determined in the method can be compared to known levels of the marker or biomarker in samples from healthy, normal controls; in a pre-infection or pre-disease sample of the subject; or in other afflicted/infected/diseased patients to establish the treated subject's disease status. For monitoring, a second level or amount of the marker or biomarker in in a sample obtained from the subject is determined at a time point later than the determination of the first level or amount, and the two marker or biomarker levels or amounts can be compared to monitor the course of disease or infection, or the efficacy of the therapy/treatment. In certain embodiments, a pre-treatment level of the marker or biomarker in the subject (e.g., in a sample obtained from the subject, etc.) is determined prior to beginning treatment as described; this pre-treatment level of marker or biomarker can then be compared to the level of the marker or biomarker in the subject after the treatment commences and/or during the course of treatment to determine the efficacy of (monitor the efficacy of) the disease treatment.

The non-naturally occurring, broadly reactive, pan-epitopic, Dengue virus antigen polypeptide, such as e.g., DENV envelope polypeptide, DENV M polypeptide, DENV prM polypeptide, DENV prM/E polypeptide, etc. as described herein, SVPs, VLPs, etc. comprising such Dengue virus antigen or immunogen polypeptides, or compositions thereof, etc. can be administered to a subject by any of the routes normally used for introducing a recombinant protein, composition containing the recombinant protein, or recombinant virus into a subject. Routes and methods of administration include, without limitation, intradermal, intramuscular, intraperitoneal, intrathecal, parenteral, such as intravenous (IV) or subcutaneous (SC), vaginal, rectal, intranasal, inhalation, intraocular, intracranial, oral, etc. Parenteral administration, such as but not limited to, subcutaneous, intravenous or intramuscular administration, is generally achieved by injection (immunization). Injectables can be prepared in conventional forms and formulations, either as liquid solutions or suspensions, solid forms (e.g., lyophilized forms, etc.) suitable for solution or suspension in liquid prior to injection, or as emulsions. Injection solutions and suspensions can be prepared from, for example, sterile powders, granules, tablets, etc. Administration can be systemic or local.

The non-naturally occurring, broadly reactive, pan-epitopic, Dengue virus polypeptides (e.g., Dengue virus envelope polypeptides, DENV M polypeptides, DENV prM polypeptides, DENV prM/E polypeptides, etc., fragments or combinations thereof, etc.) as described herein, and SVPs, VLPs, etc. comprising such DENV antigen or immunogen polypeptides, or compositions thereof, etc. can be administered to a subject in any suitable manner, such as with pharmaceutically acceptable carriers as described supra. Pharmaceutically acceptable carriers are determined in part by the particular immunogen or composition being administered, as well as by the particular method used to administer the composition. Accordingly, a pharmaceutical composition comprising the non-naturally occurring, broadly reactive, pan-epitopic, Dengue virus antigen polypeptides, such as DENV envelope polypeptides, and VLPs comprising DENV polypeptides, or compositions thereof, can be prepared using a wide variety of suitable and physiologically and pharmaceutically acceptable formulations.

Administration of the broadly reactive, pan-epitopic, Dengue virus antigen polypeptides, such as DENV envelope polypeptides, and VLPs comprising such DENV polypeptides, or compositions thereof, can be accomplished by single or multiple doses. The dose administered to a subject should be sufficient to induce a beneficial therapeutic response in a subject over time, such as to inhibit, block, reduce, ameliorate, protect against, or prevent disease or infection by Dengue virus. The dose required will vary from subject to subject depending on the species, age, weight and general condition of the subject, the severity of the infection being treated, the particular composition being used, the formulation type, the mode of administration, etc. Generally, an appropriate dose can be determined by a person skilled in the art, such as a clinician or medical practitioner, using routine experimentation.

Further provided is a method of eliciting an immune response to Dengue virus in a subject by administering to the subject a non-naturally occurring, broadly reactive, pan-epitopic, DENV protein antigen (e.g., DENV envelope protein antigen, M protein antigen, prM protein antigen, prM/E protein antigen, etc., fragments or combinations thereof as disclosed herein, fusion proteins containing the DENV protein, SVPs or VLPs containing the DENV protein, or compositions thereof as described herein. In some embodiments, the Dengue protein (e.g., DENV M protein, DENV prM protein, DENV E protein, DENV prM/E protein, etc., fragments or combinations thereof, etc.), DENV fusion protein, SVP or VLP can be administered using any suitable route of administration, such as, for example, by intramuscular injection. In some embodiments, the Dengue protein (e.g., DENV M protein, DENV prM protein, DENV E protein, DENV prM/E protein, etc., fragments or combinations thereof, etc.), fusion protein, SVP, or VLP is administered as a composition comprising a pharmaceutically acceptable carrier. In some embodiments the composition comprises an adjuvant selected from, for example, alum, Freund's complete or incomplete adjuvant, a biological adjuvant or immunostimulatory oligonucleotides (such as, e.g., CpG oligonucleotides, etc.). In other embodiments, the composition may be administered in combination with another therapeutic agent or molecule.

Also provided is a method of immunizing a subject against infection or disease or the symptoms thereof caused by Dengue virus of subtypes 1-4 or other subtypes yet to be identified or made known, in which the method involves administering to the subject SVPs or VLPs containing a non-naturally occurring, pan-epitopic, broadly reactive DENV protein (e.g., DENV M protein, DENV prM protein, DENV E protein, DENV prM/E protein, etc., fragments or combinations thereof, etc.) as described herein, or administering an immunogenic composition thereof. In some embodiments of the method, the composition further comprises a pharmaceutically acceptable carrier and/or an adjuvant. For example, the adjuvant can be alum, Freund's complete or incomplete adjuvant, a biological adjuvant or immunostimulatory oligonucleotides (e.g., CpG oligonucleotides, etc.). In an embodiment, the Dengue virus immunogens, VLPs, SVPs, compositions thereof, etc. are administered intramuscularly.

In some embodiments of the methods of eliciting an immune response or immunizing a subject against virus infection or disease caused by or associated with Dengue virus, the subject is administered at least 1 μg of the SVPs or VLPs containing a non-naturally occurring, broadly reactive, pan-epitopic DENV protein, (e.g., DENV M protein, DENV prM protein, DENV E protein, DENV prM/E protein, etc., fragments or combinations thereof, etc.), such as at least 5 μg, at least 10 μg, at least 15 μg, at least 20 μg, at least 25 μg, at least 30 μg, at least 40 μg g or at least 50 μg of the SVPs or VLPs containing the non-naturally occurring, broadly reactive, pan-epitopic Dengue virus protein, (e.g., DENV M protein, DENV prM protein, DENV E protein, DENV prM/E protein, etc., fragments or combinations thereof, etc.), for example 1 to 50 μg or 1 to 25 μg of the SVPs or VLPs containing the Dengue virus protein. In some embodiments, the subject is administered 5 to 20 μg of the SVPs or VLPs, or 10 to 15 μg of the SVPs or VLPs. In a specific, yet nonlimiting example, the subject is administered 15 μg of the SVPs or VLPs. However, one of skill in the art is capable of determining therapeutically effective amounts of SVPs or VLPs (for example, an amount that provides a therapeutic effect, protection against Dengue virus infection, etc.) suitable for administering to a subject in need of treatment or protection from virus infection.

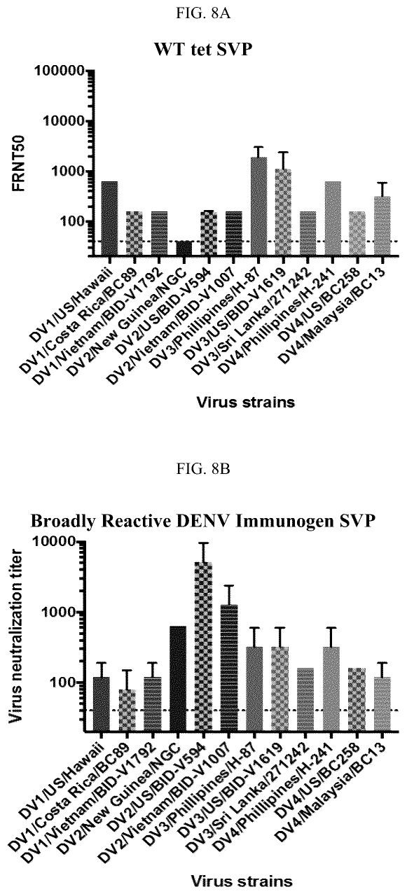

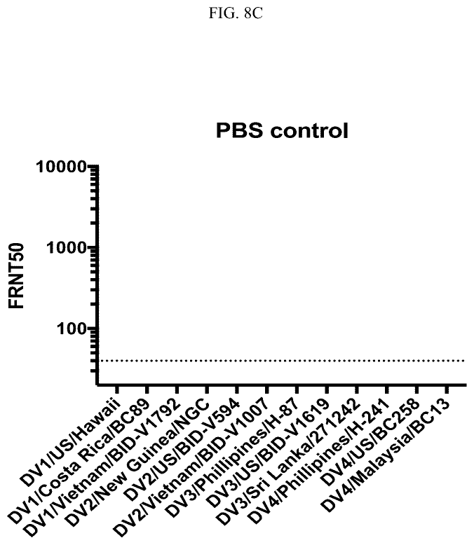

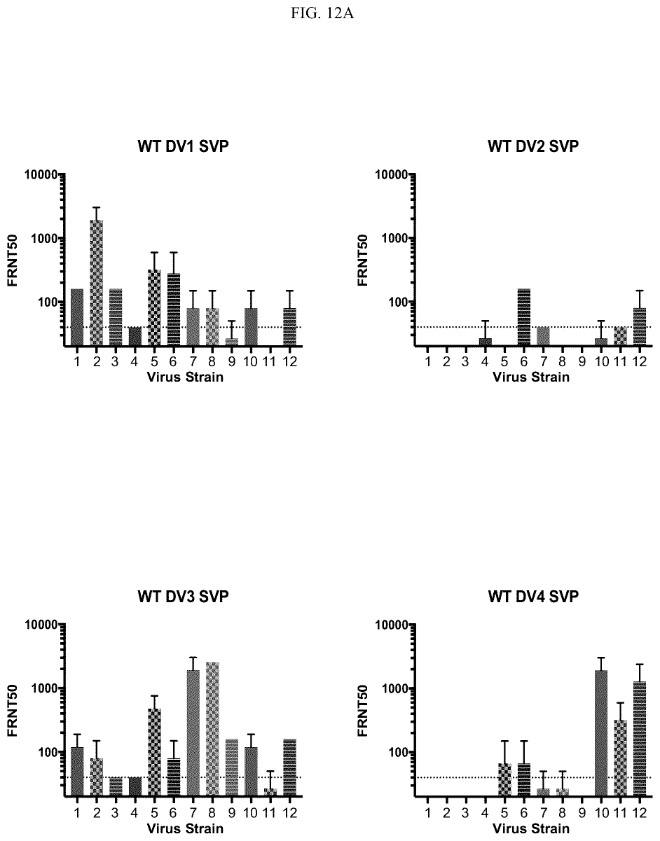

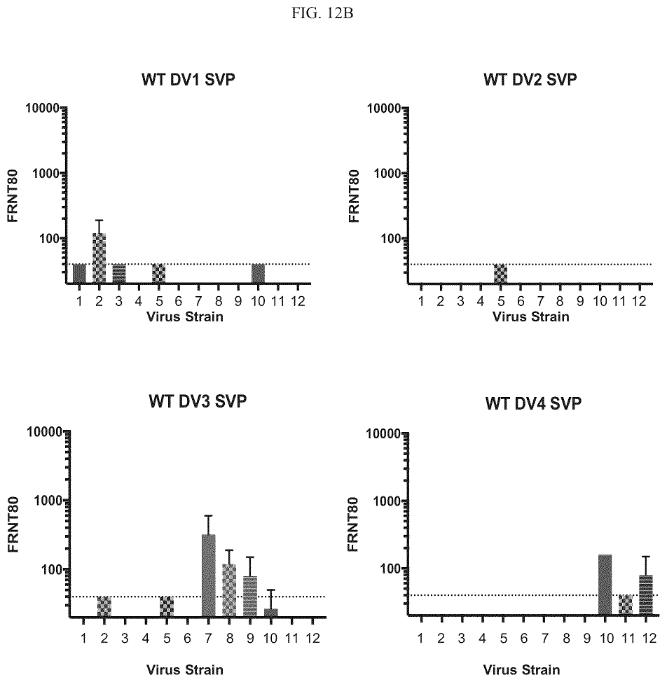

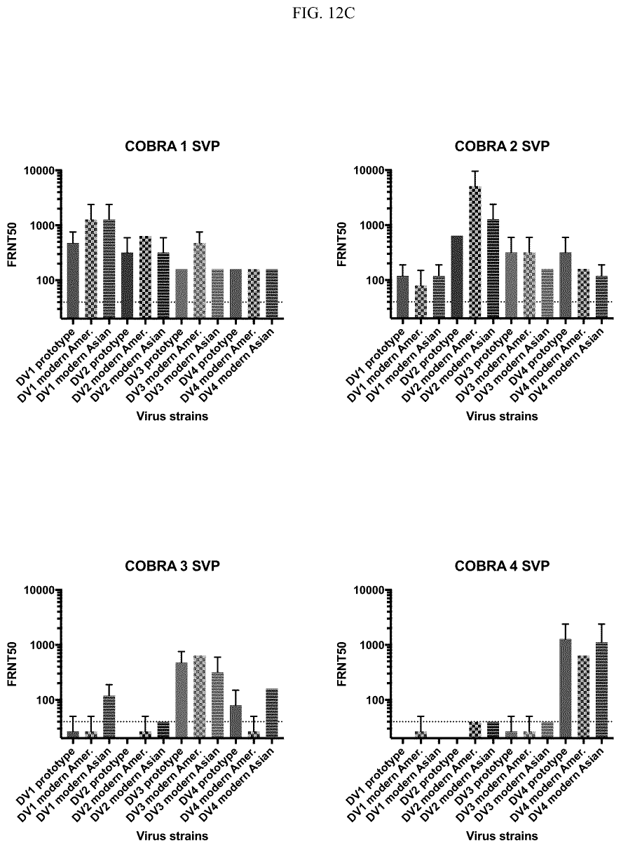

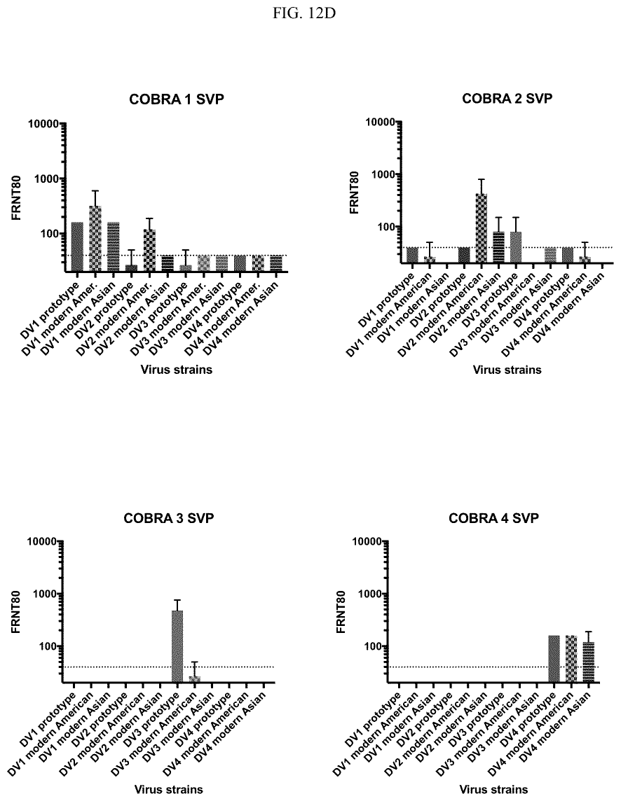

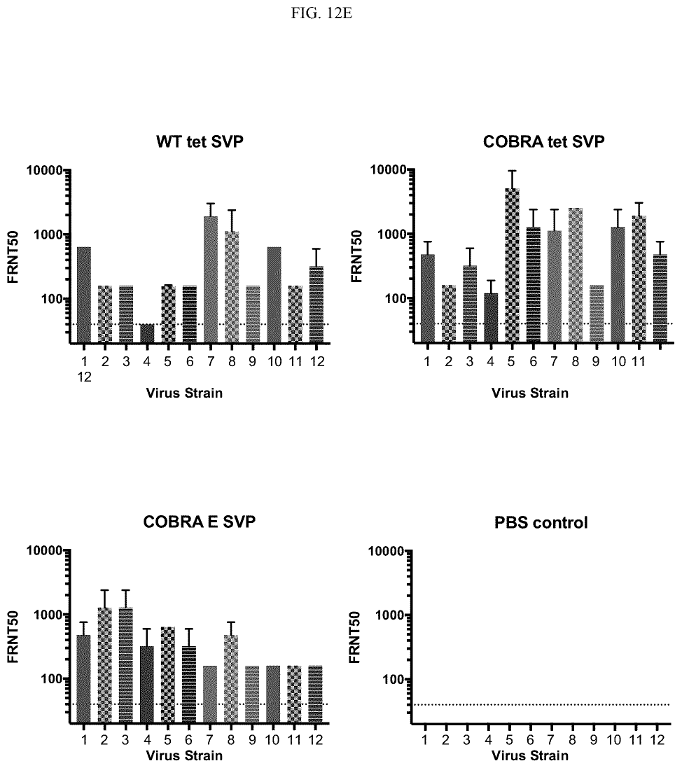

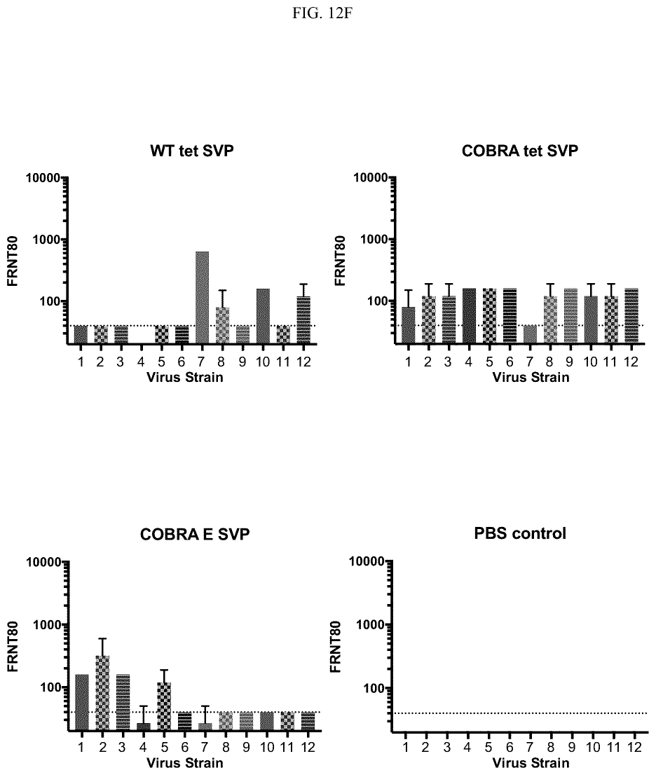

In other embodiments, the administration of SVPs or VLPs comprising a non-naturally occurring, broadly reactive, pan-epitopic Dengue virus protein, (e.g., DENV M protein, DENV prM protein, DENV E protein, DENV prM/E protein, etc., fragments or combinations thereof, etc.), or an immunogen or immunogenic composition, as described herein, may elicit high titers of neutralizing antibodies directed against the diverse repertoire of epitopic determinants on the Dengue virus protein immunogen, as well as protective levels of Dengue virus protein-inhibiting antibodies that are directed against a number of representative DENV subtypes and strains thereof, and will provide complete protection against lethal challenge with Dengue virus and/or related Dengue virus subtypes and strains thereof. The SVPs or VLPs containing a non-naturally occurring, broadly reactive, pan-epitopic Dengue virus protein, (e.g., DENV M protein, DENV prM protein, DENV E protein, DENV prM/E protein, etc., fragments or combinations thereof, etc.), as described herein elicit a broad immune response (e.g., elicit neutralizing antibodies directed against a broad range of DENV subtypes, strains, isolates, etc.) compared with the immune response elicited by a wild-type, poly-, multivalent (e.g., tetravalent, etc.) Dengue virus vaccine. (e.g., FIGS. 8A, 8B, 12A-12F).

An advantage of the immunogens and immunogenic compositions comprising non-naturally occurring, broadly reactive, pan-epitopic Dengue virus antigens (e.g., DENV M antigen, DENV prM antigen, DENV envelope antigen, DENV prM/E antigen, etc., or fragemnts or combinations thereof, etc.) described herein is that a broadly reactive immune response is elicited against not only against the Dengue virus serotype from which the antigen was derived, but also against one or more, or all, other Dengue virus serotypes or strains (e.g, DENV1, DENV2, DENV3, DENV4, ETC.). Thus, the Dengue virus immunogens are more cost effective to produce, and beneficially elicit a broadly reactive immune response, thus, obviating a need to make and administer a poly- or multi-valent immunogenic composition or vaccine.

Adjuvants and Combination Therapies

The Dengue virus immunogens or immunogenic compositions containing an Dengue virus protein antigen, (e.g., DENV M protein antigen, DENV prM protein antigen, DENV E protein antigen, DENV prM/E protein antigen, etc., fragments or combinations thereof, etc.), or containing Dengue virus SVPs or VLPs as described herein, can be administered alone or in combination with other therapeutic agents to enhance antigenicity or immunogenicity, i.e., to increase an immune response, such as the elicitation of specific antibodies, in a subject. For example, the Dengue virus SVPs or VLPs can be administered with an adjuvant, such as, but not limited to, alum, Freund's incomplete adjuvant, Freund's complete adjuvant, biological adjuvant, immunostimulatory oligonucleotides (e.g., CpG oligonucleotides, etc.), etc.

One or more cytokines, such as interleukin-1 (IL-2), interleukin-6 (IL-6), interleukin-12 (IL-12), the protein memory T-cell attractant “Regulated on Activation, Normal T Expressed and Secreted” (RANTES), granulocyte-macrophage-colony stimulating factor (GM-CSF), tumor necrosis factor-alpha (TNF-α), or interferon-gamma (IFN-γ); one or more growth factors, such as GM-CSF or granulocyte-colony stimulation factor (G-CSF); one or more molecules such as the TNF ligand superfamily member 4 ligand (OX40L) or the type 2 transmembrane glycoprotein receptor ligand belonging to the TNF superfamily (4-1BBL), etc., or combinations of these molecules, etc., can be used as biological adjuvants, if desired or warranted (see, e.g., Salgaller et al., J. Surg. Oncol. 68(2):122-38, 1998; Lotze et al., Cancer J. Sci. Am. 6(Suppl 1):561-6, 2000; Cao et al., Stem Cells 16(Suppl 1):251-60, 1998; Kuiper et al., Adv. Exp. Med. Biol. 465:381-90, 2000). These molecules can be administered systemically (or locally) to a subject.

Several ways of inducing cellular responses, both in vitro and in vivo, are known and practiced in the art. Lipids have been identified as agents capable of assisting in priming cytotoxic lymphocytes (CTL) in vivo against various antigens. For example, palmitic acid residues can be attached to the alpha and epsilon amino groups of a lysine residue and then linked (for example, via one or more linking residues, such as glycine, glycine-glycine, serine, serine-serine, or the like, etc.) to an immunogenic peptide (see, e.g., U.S. Pat. No. 5,662,907, etc.). The lipidated peptide can then be injected directly in a micellar form, incorporated in a liposome, or emulsified in an adjuvant. As another example, E. coli lipoproteins, such as tripalmitoyl-S-glycerylcysteinlyseryl-serine can be used to prime tumor-specific CTL when covalently attached to an appropriate peptide (see, e.g., Deres et al., Nature 342:561, 1989, etc.). Moreover, the induction of neutralizing antibodies can also be primed with the same molecule conjugated to a peptide which displays an appropriate epitope, and two compositions can be combined to elicit both humoral and cell-mediated responses where such a combination is deemed desirable.

While treatment methods may involve the administration of SVPs or VLPs containing a non-naturally occurring, broadly reactive, pan-epitopic Dengue virus immunogenic protein, (e.g., DENV E protein, DENV M protein, DENY prM protein, DENV prM/E protein, etc., or fragments or combinations thereof, etc.), as described herein, one skilled in the art will appreciate that the non-naturally occurring, broadly reactive, pan-epitopic Dengue virus protein itself (in the absence of a viral particle), as a component of a pharmaceutically acceptable composition, or as a fusion protein, can be administered to a subject in need thereof to elicit an immune response against Dengue virus in the subject.

Kits

Also provided are embodiments of the disclosure directed to kits containing a non-naturally occurring, broadly reactive, pan-epitopic Dengue virus immunogen or composition (e.g., vaccine, pharmaceutically acceptable composition, immogenic composition, medicinal product, etc.) containing the immunogen or antigen as described herein, and a pharmaceutically acceptable carrier (e.g., diluent, excipient, etc.) for administering to a subject, for example. The immunogen may be in the form of a Dengue virus protein (polypeptide) or a polynucleotide (a polynucleotide encoding a Dengue virus polypeptide (e.g., DENV M protein, DENV prM protein, DENV E protein, DENV prM/E protein, etc., or fragments or combinations thereof, etc.), as described herein. Kits containing one or more of the plasmids, or a collection of plasmids as described herein, are also provided. As will be appreciated by the skilled practitioner in the art, such a kit may contain one or more containers that house the immunogen, vaccine, composition, carriers, diluents, excipients, etc., as necessary, and instructions for use.

Recombinant Polypeptide Expression

The practice of the present disclosure employs, unless otherwise indicated, conventional techniques of molecular biology (including, for example, recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Useful techniques for particular embodiments will be discussed in the sections that follow.

The following examples are put forth to provide those of ordinary skill in the art with a complete disclosure and description of how to make and use the assay, screening, and therapeutic methods of embodiments of the invention, and are not intended to limit the scope of what the inventors regard as their invention.

### EXAMPLES

The following examples are provided to illustrate certain particular features and/or embodiments. The examples should not be construed to limit the disclosure to the particular features or embodiments described.

### Example 1

Antibody Responses To Broadly Reactive Dengue Virus (Deny) Envelope (E) Protein Immunogens

Broadly reactive DENV E protein antigen sequences and wild-type DENV E protein sequences were used to produce subviral particles (SVPs), which were expressed from 293T cells using a mammalian expression vector containing polynucleotides encoding prM-E protein. The DENV serotypes and virus strains used are presented in Table 1.

TABLE 1

| Serotype | Type | Virus strain |
|---|---|---|
| DV1 | Prototype | DV1/US/Hawaii/1944 |
| DV1 | Modern American | DV1/Costa Rica/BC89/1994 |
| DV1 | Modern Asian | DV1/Vietnam/BID-V1792/2007 |
| DV2 | Prototype | DV2/New Guinea/NGC/1944 |
| DV2 | Modern American | DV2/US/BID-V594/2006 |
| DV2 | Modern Asian | DV2/Vietnam/BID-V1007/2006 |
| DV3 | Prototype | DV3/Philippines/H-87/1956 |
| DV3 | Modern American | DV3/US/BID-V1619/2005 |
| DV3 | Modern Asian | DV3/Sri Lanka/271242/1991 |
| DV4 | Prototype | DV4/Philippines/H-241/1956 |
| DV4 | Modern American | DV4/US/BC258/1994 |
| DV4 | Modern Asian | DV4/Malaysia/BC13/1997 |

SVPs were prepared by methods used in the art, for example, as described by Skup, D. et al., J. Virol., 34(2):490-496, 1980; and Graves, P. N. et al., Virology, 126(1):106-116, 1983.

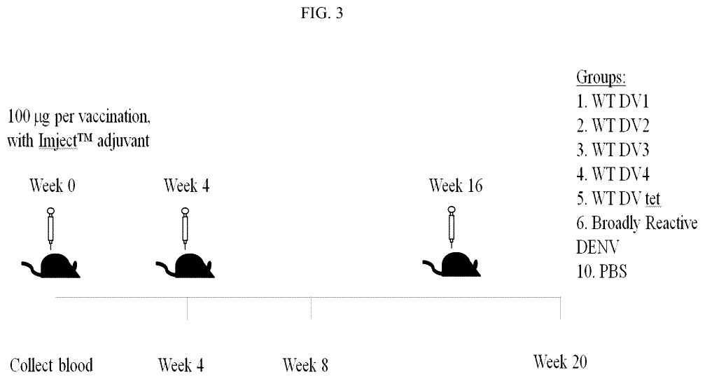

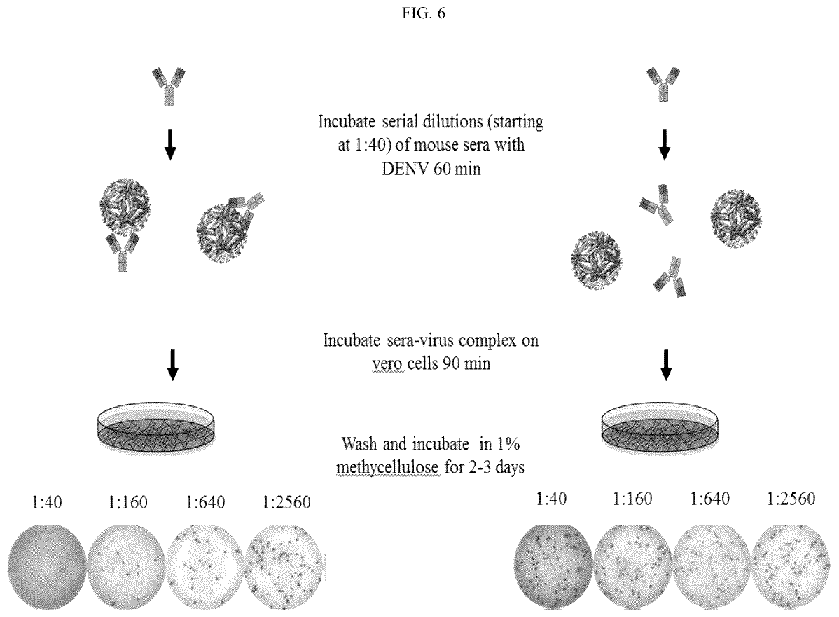

Female C57BL/6 mice (age 6-8 weeks) were immunized (vaccinated) intramuscularly with the SVPs (wild type DENV SVPs or broadly reactive DENV immunogen SVPs) three times at 4 week intervals with 100 μg total SVP plus IMJECT™ Alum. (FIG. 3). Vaccines were prepared as individual or tetravalent SVP formulations. Immune sera were collected from the immunized animals and total IgG antibody titers to DENV E were analyzed by ELISA. The ability to inhibit or block virus infection in vitro was assessed in a focus reduction neutralization test (FRNT), (FIG. 6), against a panel of 12 prototype and modern strains from all four serotypes. The results (FRNT₅₀) or the assay are shown in Table 2 below.

TABLE 2

| Immunogen | Number of strains | Number of strains |
|---|---|---|
| Administered | at 1:40 (out of 12) | at 1:100 |
| WT DV1 | 9 | 5 |
| WT DV2 | 1 | 1 |
| WT DV3 | 9 | 7 |
| WT DV4 | 5 | 3 |
| WT tet | 11 | 11 |
| Broadly reactive | 12 | 11 |
| DENV immunogen | | |

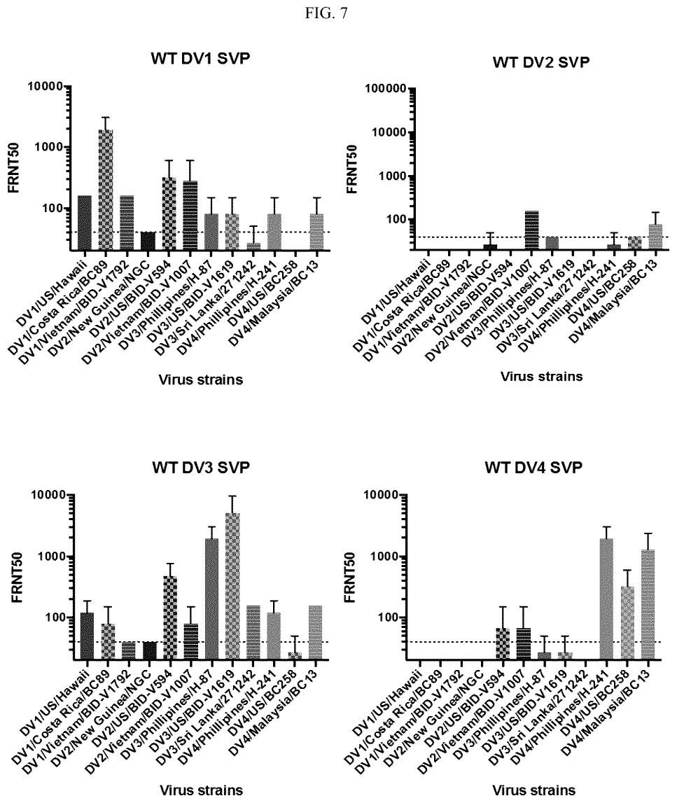

Mice immunized with wild type DENV SVPs produced anti-E IgG antibodies that were specific to strains in each homologous serotype. The elicited antibodies neutralized serotype specific viruses. The broadly reactive DENV immunogen SVPs as described herein elicited a broader breadth of antibodies that neutralized various DENV strains across all four serotypes of DENV (DENV1-4). The DENV E immunogen as described herein neutralized all 12 strains of DENV in vitro, comparable to tetravalent SVP immunization and a resulting broadly reactive immune response. (FIGS. 4, 5, 7 and 8A-C).

### Example 2

Focus (Plaque) Reduction Neutralization Test (Frnt)

A focus (plaque) reduction neutralization test was used to quantify the titer of neutralizing antibody directed against a virus or immunogenic virus antigen. The serum sample or solution of antibody to be tested was diluted and mixed with a viral suspension, and the mixture was incubated to allow an antibody-virus interaction. Thereafter, the mixture was added to a confluent monolayer of host cells. The surface of the cell layer was covered in a layer of agar or carboxymethyl cellulose to prevent nonspecific virus spreading. The concentration of plaque forming units was estimated by the number of plaques (regions of infected cells) formed after a few days. Depending on the virus, the plaque forming units are measured by microscopic observation, fluorescent antibodies or specific dyes that react with infected cells.https://en.wikipedia.org/wiki/Plaque_reduction_neutralization_test—cite_note-schimidt1976-1 See, e.g., Thomas, Stephen J. et al., 2009, “Dengue Plaque Reduction Neutralization Test (PRNT) in Primary and Secondary Dengue Virus Infections: How Alterations in Assay Conditions Impact Performance,” The American Journal of Tropical Medicine and Hygiene, 81 (5): 825-833, 2009. The concentration of serum needed to reduce the number of plaques by 50% compared to the serum-free virus provides a measure of the quantity of antibody that is present or how effective the antibody is at binding the virus/antigen. This measurement is called the PRNT₅₀ value.

Currently, FRNT is considered the “gold standard” for detecting and measuring antibodies that can neutralize the viruses that cause many diseases. The assay has a higher sensitivity than other tests, such as hemagglutination of other commercial enzyme-based assays, without compromising specificity. Moreover, FRNT is more specific than other serological methods for the detection of some arboviruses. However, the test has a duration of a few days relative to EIA kits that provide results more quickly (e.g., within minutes to a few hours).

In the FRNT assay, the neutralization ability of the antibodies can depend on the virion maturation state and the cell type used. See, e.g., Mukherjee, S. et al., “Mechanism and Significance of Cell Type-Dependent Neutralization of Flaviviruses,” J. Virol., 88 (13):7210-7220, 2014. Thus, the correct cell line should be used for the assay to avoid a risk of equivocal results.

### Example 3

Antibody Binding To Dengue Virus (Deny) Envelope (E) Protein By Enzyme Linked Immunosorbent Assay (ELISA)

An Enzyme Linked Immunosorbent Assay (ELISA) utilizes a specific antibody linked to an enzyme to detect the presence of an unknown amount of antigen (e.g., virus) in a sample. The antibody-antigen binding event is detected and/or quantified through the enzyme's ability to convert a reagent to a detectable signal that can be used to calculate the concentration of the antigen in the sample. (See, e.g., Kemeny, D. M. and Challacombe, S. J., 1988, ELISA and Other Solid Phase Immunoassays: Theoretical and Practical Aspects. John Wiley and Sons. ISBN0-471-90982-3). Horseradish peroxidase (HRP) is a commonly employed enzyme in ELISA protocols due to its ability to amplify signal and increase assay sensitivity. ELISA assays can generally be classified as either indirect, competitive, sandwich or reverse. ELISA kits are commercially available for use according to the methods. Quantification is typically performed using chromogenic reporters or fluorescence (e.g. Invitrogen, Santa Cruz Biotechnology Inc.). The performance of an ELISA may be completed in about 4 to 24 hours depending on antibody incubation time.

### Example 4

Mouse Studies

C57BL/6 mice (Mus musculus, 6-8 weeks of age) were purchased from Jackson Laboratory (Bar Harbor, ME, USA), housed in microisolator units and allowed free access to food and water. The animals were cared for under University of Georgia Research Animal Resources guidelines for laboratory animals. All procedures were reviewed and approved by the Institutional Animal Care and Use Committee (IACUC). Mice (typically 5 mice per group) were vaccinated with purified DENY immunogen SVPs or VLPs, (3.0 μg/mouse), based upon protein content from ELISA quantification, and SVP or VLP vaccines were delivered to the animals via intramuscular injection at week 0. Animals were boosted with the same immunogen (vaccine) at the same dose, e.g., at weeks 4 and 8. The immunogenic compositions (vaccines) at each dose were formulated with an emulsified squalene-in-water adjuvant (Sanofi Pasteur, Lyon, France). The final concentration after mixing 1:1 with SVPs or VLPs was 2.5% squalene. Twenty-eight days after each vaccination, blood samples were collected via the submandibular cheek, and the samples were transferred to a microcentrifuge tube. The tubes were centrifuged at 10,000 rpm for 10 minutes. Serum samples were removed and frozen at −20° C.±5° C.

### Example 5

Generation of Cobra Constructs and Svps

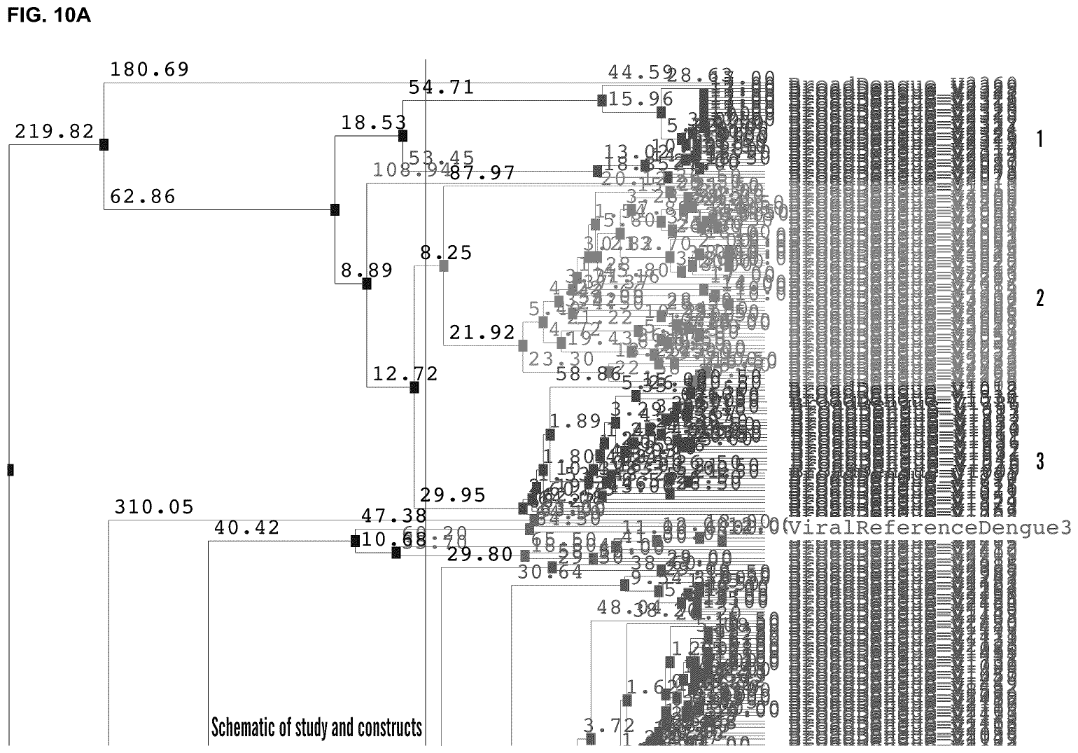

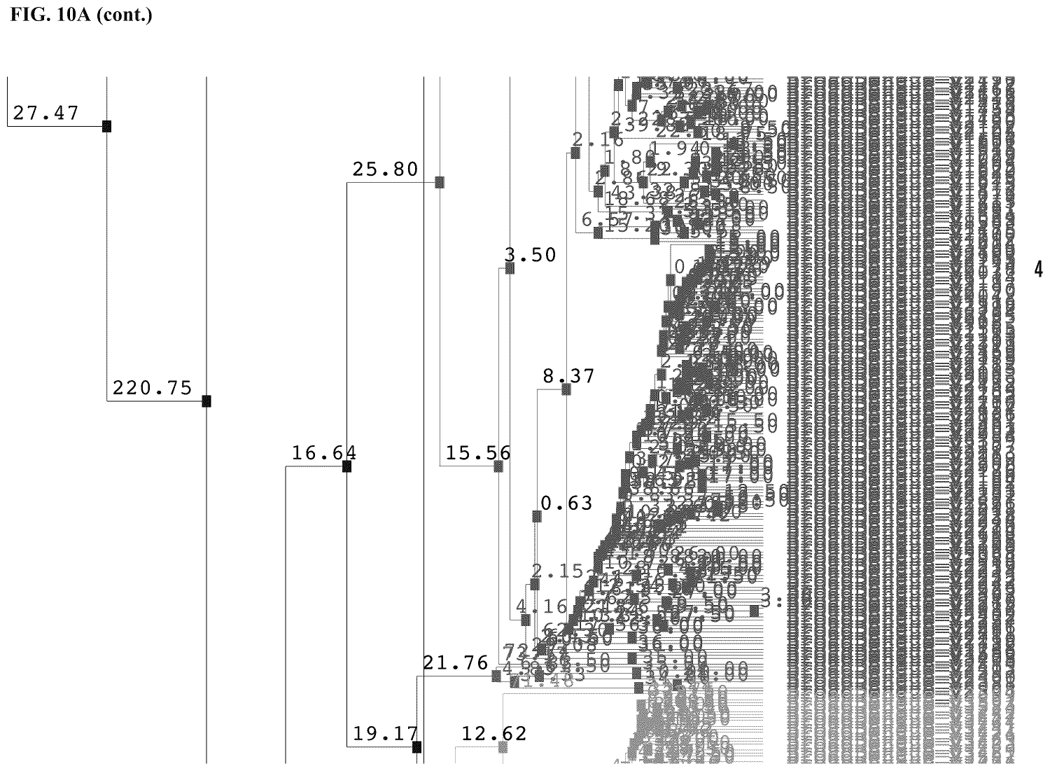

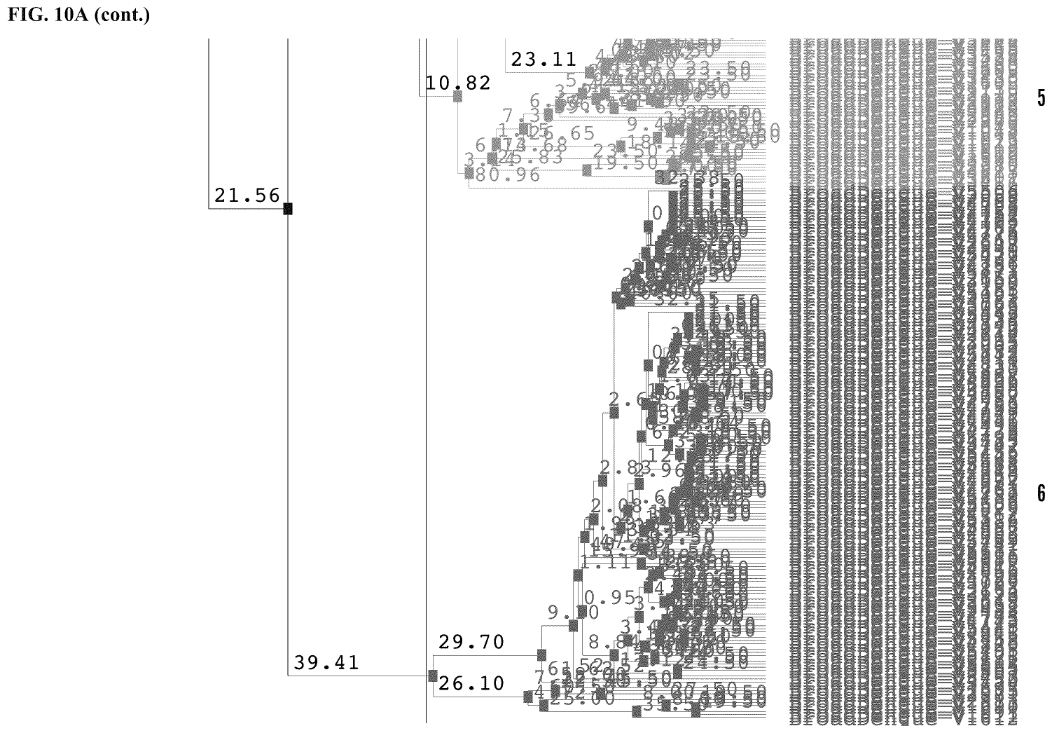

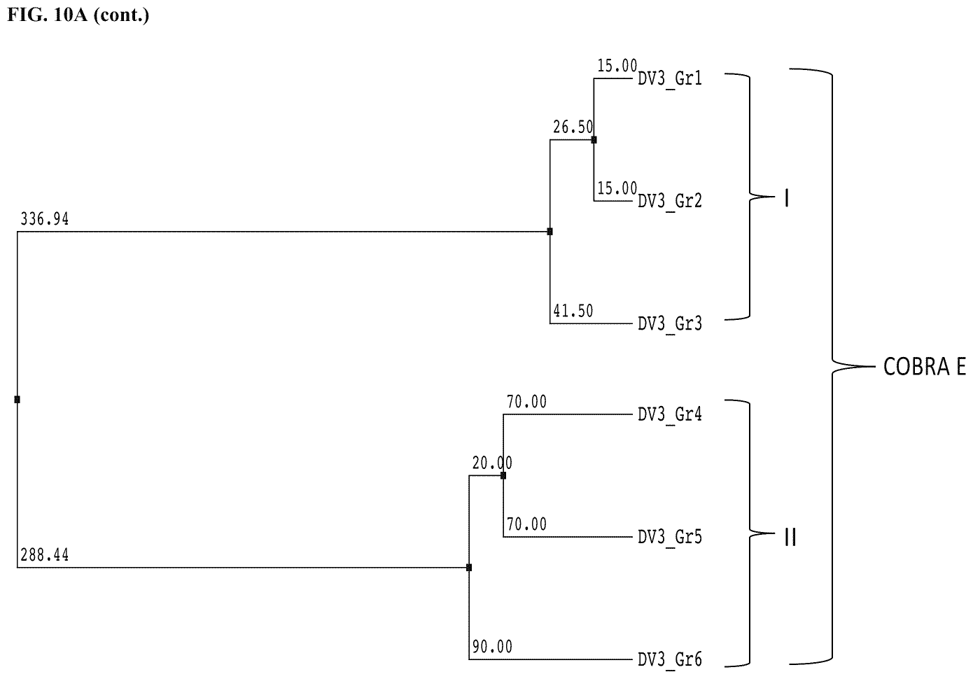

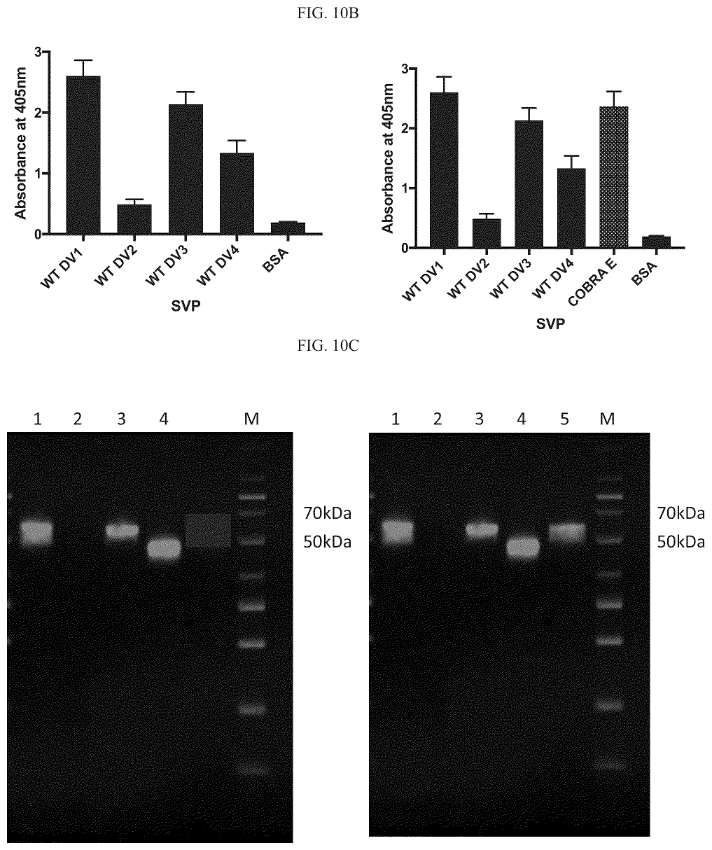

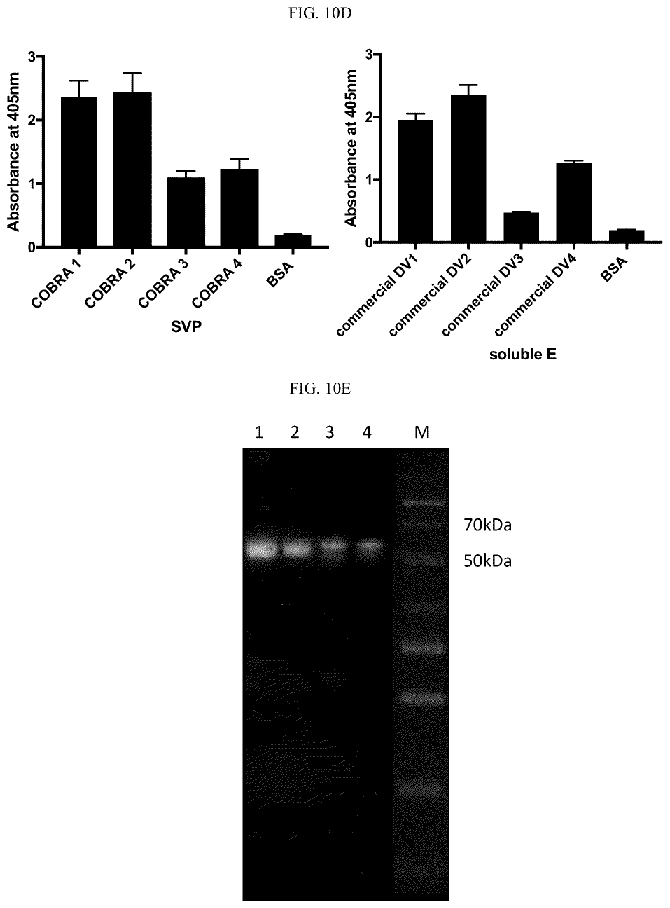

Dengue virus envelope (E) nucleotide sequences from infected human were downloaded from the GenBank Database (www.ncbi.nlm.nih.gov/genbank), and a multilayered process was used to generate DENV E COBRA sequences (FIG. 10A) based on sequence similarity and/or identity. The sequences were clustered based on similarity, and six primary sequences were created. Two secondary sequences were made from the primary sequences, and four final COBRA DENV sequence were derived. Plasmids or mammalian expression vectors encoding the preMembrane-Envelope (prM-E) genes representing COBRA or wild type strains from all 4 serotypes were constructed and transiently transfected into human embryonic kidney (HEK) 293T cells (which have a temperature-sensitive mutant of the SV40 large T antigen). COBRA and wild-type SVPs were expressed from these cells.

Verification of purified SVPs were done by ELISA and Western Blot (FIGS. 10B-10E). Pan-Dengue E monoclonal antibody (mAb) bound to purified SVPs coated on ELISA plates, though at different levels. The Western blot showed bands at ˜50-60 kDa for the monomeric E protein. WT DV2 SVP had minimal binding, though protein concentration was similar to the other SVPs when measured by bicinchoninic acid protein assay (data not shown). This may be due to a detection issue, since commercial Dengue E proteins also had different levels of monoclonal (mAb) binding on ELISA plates (FIG. 10D) and could not be detected at all on Western Blot (data not shown). Additional COBRA sequences created for the COBRA tetravalent formulation were detected by ELISA and Western Blot (FIG. 10D; FIG. 10E).

In more detail, for the ELISA, Nunc Maxisorp plates (Thermo Fisher Scientific) were coated overnight at 4° C. with SVPs diluted in PBS (1 μg/mL). Plates were washed with PBS containing 0.05% Tween (PBS-T) and blocked with 1% BSA in PBS for 1 hour at room temperature. Subsequently, pan-dengue monoclonal antibody (ACRIS antibody AM01108PU-N, OriGene Technologies, Inc. Rockville, MD, USA) were added to the plates for 2 hours. After washing 3 times, peroxidase-conjugated anti-mouse IgG antibody was added for 1 hour before developing the plates using a p-nitrophenyl phosphate (pNpp) substrate (SeraCare, Milford, MA, USA). The optical density (O.D.) was determined at 405 nm. For the Western Blot, of purified SVPs was added to a Bolt™ 4-12% Bis-Tris gel (Thermo Fisher Scientific) for SDS-PAGE under non-reducing conditions and electroblotted on a polyvinylidene difluoride (PVDF) membrane. The samples were probed with pan-flavivirus monoclonal antibody (mAb) (5E284, Santa Cruz Biotechnology, Dallas TX, USA) using the iBind™ Western Device (Thermo Fisher Scientific). Goat anti-mouse IgG conjugated to horseradish peroxidase (HRP) (Southern Biotech, Birmingham, AL, USA) was used as the secondary antibody. Clarity™ Western enhanced chemiluminescence (ECL) substrate (Bio-Rad, Hercules, CA, USA) was used for chemiluminescent imaging.

The full-length pre-membrane/envelope (prM/E) gene segments (˜2,100 bp) were cloned in the pTR600 expression plasmid, as previously described (Ross, T. et al. J Vaccine Immunotechnology 1(1):7, 2014; Giles B M & Ross T M. Vaccine 29:3043-3054, 2011). Briefly, dengue virus (DENV) E nucleotide sequences isolated from human infections from 1941 to 2006 were downloaded from the GenBank Database (www.ncbi.nlm.nih.gov/genbank) and the sequences were translated into protein sequences (100 from each serotype). DENV E amino acid sequences were group by phylogenetic clustering and geographic location per serotype. Multiple rounds of sequence alignments were performed to retain common epitopes within the sequence. This methodology is referred to computationally optimized broadly reactive antigen (COBRA) design and has been described previously (See, e.g., Crevar C J, et al. Hum Vaccin Immunother 11:572-583, 2015; Giles B M, et al. Clin Vaccine Immunol. 19:128-139, 2012; Giles B M, et al. J Infect Dis. 205:1562-1570, 2012; Giles B M and Ross T M. 2011. Vaccine 29:3043-3054, 2016; Carter D M, et al. J. Virol. 90(9):4720-4734, 2016). The final gene sequences were aligned using Align X (Vector NTI). The final amino acid COBRA E sequences, as well as the wild-type E sequences from prototype, wild-type (WT) DENV were reverse translated and optimized for expression in mammalian cells, including codon usage and RNA optimization (Gene Art; Regensburg, Germany). Prototype virus E sequences from the Hawaii (DENV-1), NCG (DENV-2), H87 (DENV-3), and H241 (DENV-4); (accession numbers X76219, M29095, M93130, S66064) were optimized. WT and COBRA DENV E sequences representing all four subtypes of dengue were constructed. Plasmids were amplified using QIAGEN® plasmid maxi kit, according to the manufacture's protocol (Qiagen, Germantown, MD, USA). Plasmids were verified using restriction enzyme digestions and examination of specific DNA fragment patterns following resolution by electrophoresis using on a 1% agarose gel.

Human embryonic kidney (HEK) 293T cells (10⁷ in T150 flasks) were transfected with 20 μg of each DNA plasmid using Lipofectamine® 3000 according to the manufacturer's instructions (Thermo Fisher Scientific Waltham, MA, USA). After incubating for 4 days at 37° C., supernatants were collected and cell debris removed by centrifugation and vacuum filtration through a 0.22 μM sterile filter. Subviral particles (SVPs) were collected via ultracentrifugation (100,000×g through 20% glycerol wt/vol) for 4 hours at 4° C. The concentrated SVP pellets were subsequently resuspended in phosphate buffered saline (PBS) and stored at −80° C. Protein concentration was determined by MicroBCA™ Protein Assay Reagent Kit (Thermo Fisher Scientific).

### Example 6

Seroconversion of Deny Svp Vaccine Groups

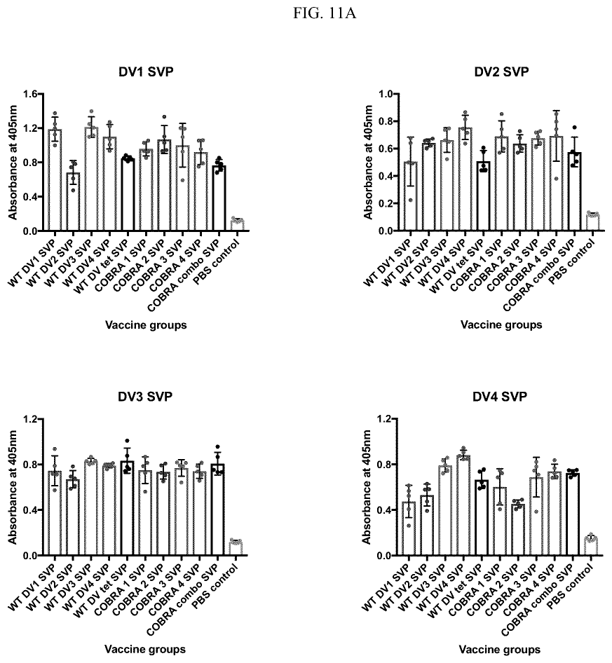

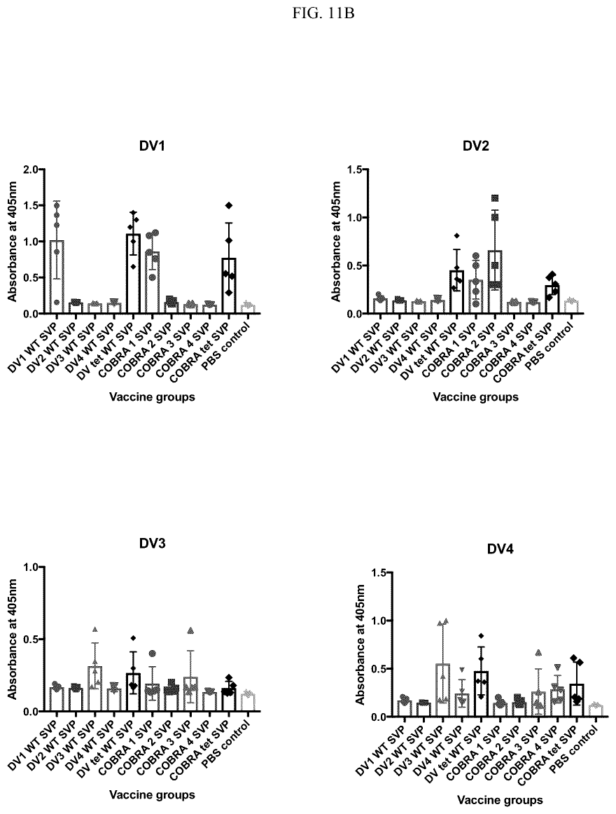

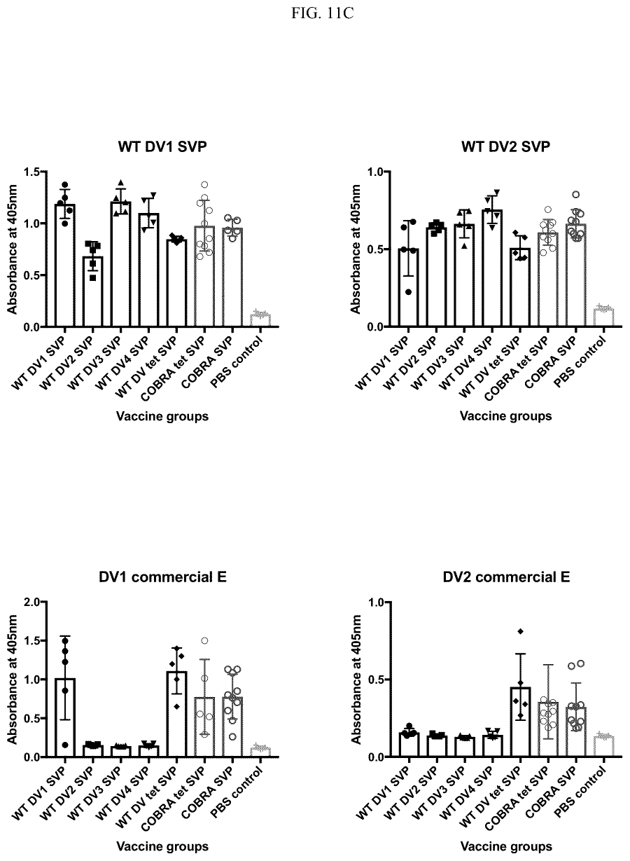

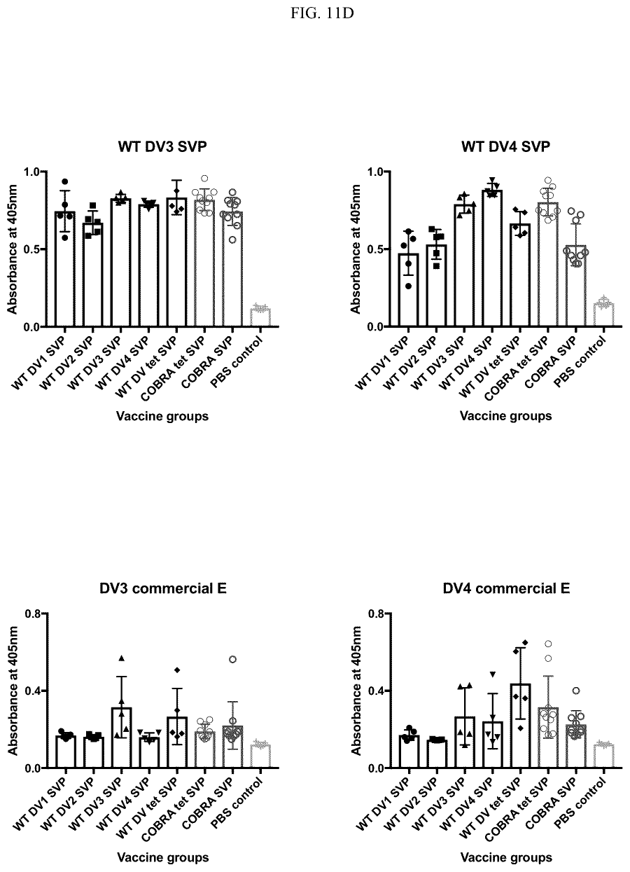

C57BL/6 mice were given WT and COBRA SVP vaccinations in monovalent or tetravalent formulations. Total IgG binding of sera to in-lab SVP or commercial E representing all four serotypes were calculated (FIG. 11). WT DV1-4 made in-lab produced broad cross reactivity in all groups (FIGS. 11A-11B). Commercial soluble E DV1-4 had narrow, homologous reactivity (FIGS. 11A-11B).

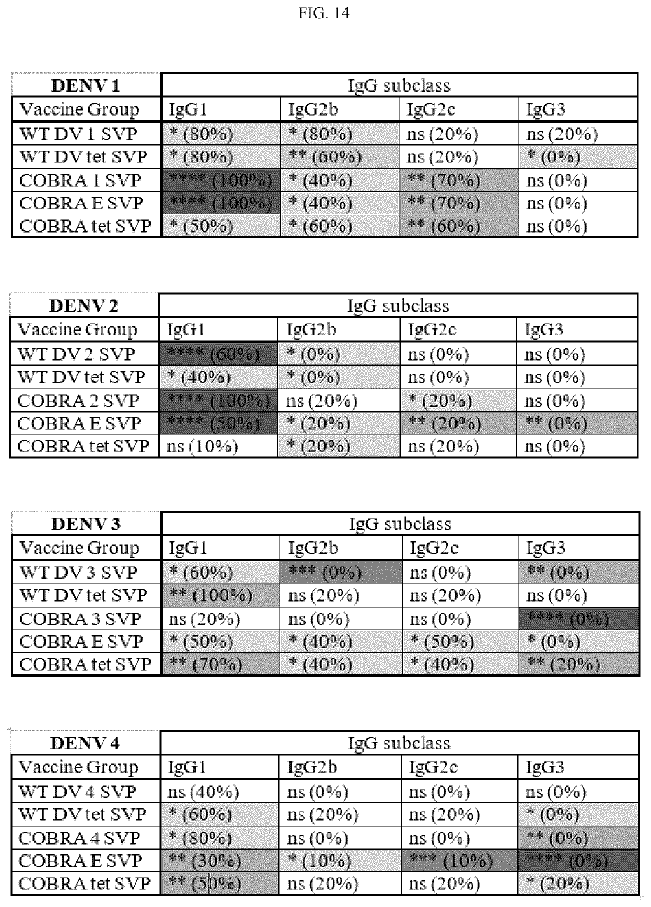

IgG subclasses elicited against Dengue E were also analyzed for homotypic WT SVP, COBRA E SVP, and tetravalent SVP groups (FIG. 14). Statistical differences were measured against PBS control groups, with the color intensity increasing with the significance. All vaccinations elicited some level of IgG1, though some were not statistically significant. COBRA E SVP groups elicited significant levels of IgG2b, IgG2c towards all dengue serotypes.

Mouse vaccination occurred as described in more detail. Female C57BL/6 mice (age 6-8 weeks, n=5 for wild type groups, n=10 for COBRA groups) were administered wild type (WT) or COBRA SVPs by intramuscular (i.m.) injection with at weeks 0, 4 and 16 (100 μg of SVP protein per vaccination, PBS was used as mock vaccination) with Imject™ alum adjuvant (Thermo Fisher Scientific, Waltham, MA, USA). Blood was collected on weeks 4, 8, and 20 post-vaccination, then centrifuged at 6000 rpm for 10 min to separate the serum. Sera were frozen at −80° C.

Statistical Analysis demonstrated differences in IgG subclass response that were analyzed by unpaired t-test with Welch's correction. Differences in neutralization titer were analyzed by paired student's t-test. Statistical analyses were performed using Prism (GraphPad Software, La Jolla, CA, USA). P-values less than 0.05 were considered statistically significant. ns for P>0.05, * for P≤0.05, ** for P≤0.01, *** for P≤0.001, **** for P≤0.0001.

### Example 7

Neutralizing Antibody Titers

Vaccine effectiveness was analyzed against a panel of Dengue viruses representing all four serotypes from prototype and modern strains. Monovalent WT vaccine groups had higher FRNT₅₀ to homologous strains compared to heterologous strains (FIG. 12A). This was more evident in the FRNT₈₀ calculations (FIG. 12B). Tetravalent formulations of WT and COBRA had neutralizing antibody titers against all strains, with tetravalent COBRA being more robust than WT (FIG. 12E; FIG. 12F). The single COBRA E SVP vaccination also elicited neutralizing antibodies against all strains (FIG. 12E; FIG. 12F).

The neutralization assay was conducted as described here. Neutralization activity in collected antisera was determined using a focus reduction neutralization test (FRNT). Sera were heat inactivated and serially diluted (4 fold). Sera were incubated with dengue viruses in viral titers calculated as fluorescent focus units (FFU) per ml (100 FFU) representing each of the 4 serotypes for 60 min at 37° C. Three dengue viruses were used for each serotype which was represented by the prototype, modern Asian, and modern American strains. Each dengue virus was mixed with the serially diluted antisera and used to infect a single cell monolayer of vero cell in 96-well plates for 90 min at 37° C. that were plated. Cells were overlayed methylcellulose (1% [wt/vol]). Cells were incubated for 3 days at 37° C. (2 days for DENV 4 viruses). Cells were washed with PBS and fixed with a mixture of acetone and methanol (1:1). Foci were stained using Vectastain ABC kit, following the manufacturer's protocols (Vector Laboratories, Burlingame, CA, USA). Anti-DENV envelope monoclonal antibodies were used to detect virally infected cells. For DENV 1 and 3 viruses, the antibody AM01108PU-N (OriGene Technologies, city, state, USA) was used and for DENV 2 the monoclonal antibody 9.F.10 (Santa Cruz Biotechnology, Dallas, TX, USA) was used and for DENV 4, the pan-flavivirus monoclonal antibody 4G2 mAb (Millipore Sigma, Billerica, MA, USA) was used. Foci were imaged using CTL Immunospot (Cellular Technology Limited, Cleveland, OH, USA). FRNT-50 determined by calculating the point at which sera reduced dengue foci by 50% compared to serum-free, dengue virus control wells. Similarly, FRNT-80 determined by calculating the point at which sera reduced dengue foci by 80% compared to serum-free, dengue virus control wells.

Quantitative ELISA was performed to detect anti-SVP or anti-E antibodies elicited by vaccination. Nunc Maxisorp plates were coated (2 ug/mL) overnight at 4° C. with either subviral particles expressing one of the wild-type DENV E proteins or soluble DENV E (either DENV 1-4). Plates were washed with PBS containing 0.05% Tween (PBS-T) and blocked with 1% BSA in PBS for 1 hour at room temperature. Subsequently, 1:100 dilutions of sera samples were added to the plates for 2 h (for the positive control, sera from a separate tetravalent DENV mRNA vaccine study was used). Following three washes with PBS-T, plates were coated with peroxidase-conjugated anti-mouse IgG-Fc, IgG1, IgG2b, IgG2c, or IgG3 antibody (Bethyl Laboratories, Montgomery, TX, USA) was added for 1 hour before developing the plates using an pNpp substrate. OD values were read at 405 nm.

Dengue viruses used in this study were for DENV 1: DV1/US/Hawaii/1944, DV1/Vietnam/BID-V1792/2007, DV1/Costa Rica/BC89/1994; DENV 2: DV2/New Guinea/NGC/1944, DV2/Vietnam/BID-V1007/2006, DV2/US/BID-V594/2006; DENV 3: DV3/Phillipines/H-87/1956, DV3/Sri Lanka/271242/1991, DV3/US/BID-V1619/2005, and DENV 4: DV4/Phillipines/H-241/1956, DV4/Malaysia/BC13/1997 DV4/US/BC258/1994.

### Example 8

Longevity of Breadth of Neutralizing Antibodies

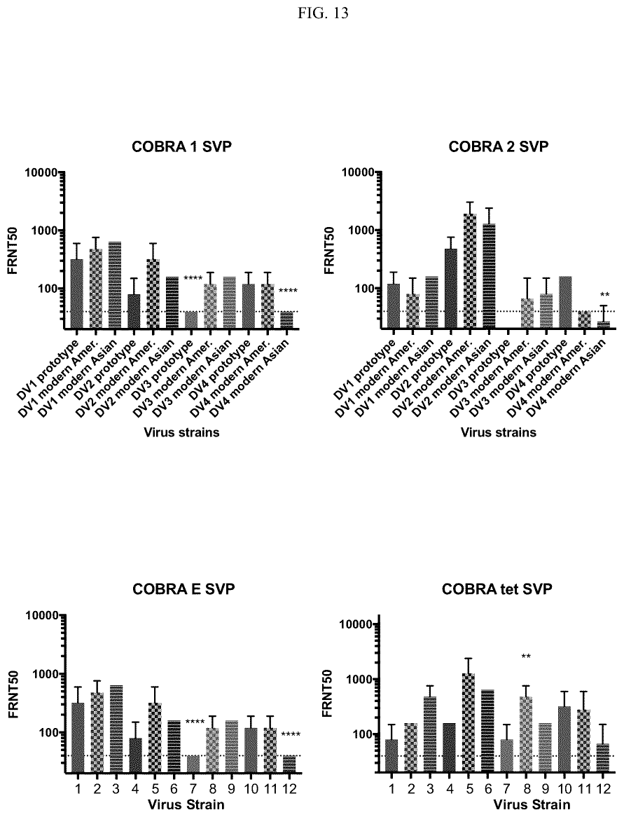

Five months after the final vaccination, the COBRA groups still maintained the breadth of neutralizing antibodies, though the titers significantly decreased for 2 strains in COBRA 1 group (i.e., Virus Strains DV3 prototype and DV4 modern Asian), 1 strain for COBRA 2 (i.e., Virus Strain DV4 modern Asian), 2 strains in COBRA E groups (i.e., DV3 prototype (Lane 7) and DV4 Modern Asian (Lane 12)), and 1 strain for COBRA tetravalent (i.e., DV3 Modern Amer. (Lane 8)). The Virus Strains are as follows: Lanes 1) DV1 prototype; 2) DV1 Modern Amer.; 3) DV1 Modern Asian; 4) DV2 prototype; 5) DV2 Modern Amer.; 6) DV2 Modern Asian; 7) DV3 prototype; 8) DV3 Modern Amer.; 9) DV3 Modern Asian; 10) DV4 prototype; 11) DV4 Modern Amer.; 12) DV4 Modern Asian. See, e.g., FIG. 13.

### Other Embodiments

Specific Embodiment 1. A non-naturally occurring, broadly reactive, pan-epitopic antigen of Dengue virus that generates an immune response against one or more Dengue virus subtypes.

Specific Embodiment 2. The Dengue virus antigen of Specific Embodiment 1, wherein the antigen comprises a Dengue virus envelope (E) protein, a Dengue virus precursor membrane (prM/E) protein, or an antibody-binding portion thereof.

Specific Embodiment 3. The Dengue virus antigen of Specific Embodiment 1 or Embodiment 2, wherein the one or more Dengue virus subtypes is selected from the group consisting of: DENV1, DENV2, DENV3, DENV4, and any combination(s) thereof.

Specific Embodiment 4. The Dengue virus antigen of any one of Specific Embodiments 1-3, which comprises an amino acid sequence that is at least 90% identical to an amino acid sequence of a DENV antigen as set forth in FIGS. 1A, 1B, 9A, 9B, or SEQ ID NOs:1-11.

Specific Embodiment 5. The Dengue virus antigen of any one of Specific Embodiments 1-3, which comprises an amino acid sequence that is at least 98% identical to an amino acid sequence of a DENV antigen as set forth in FIGS. 1A, 1B, 9A, 9B, or SEQ ID NOs:1-11.

Specific Embodiment 6. The Dengue virus antigen of any one of Specific Embodiments 1-3, which comprises an amino acid sequence of a DENV antigen as set forth in FIGS. 1A, 1B, 9A, 9B, or SEQ ID NOs:1-11.

Specific Embodiment 7. A virus-like particle (VLP) comprising the Dengue virus antigen of any one of Specific Embodiments 1-6.

Specific Embodiment 8. The VLP of Specific Embodiment 7, which comprises a polynucleotide encoding the Dengue virus antigen.

Specific Embodiment 9. A subviral particle (SVP) comprising the Dengue virus antigen of any one of Specific Embodiments 1-6.

Specific Embodiment 10. The SVP of Specific Embodiment 9, which comprises a polynucleotide encoding the Dengue virus antigen.

Specific Embodiment 11. A non-naturally occurring, pan-epitopic immunogen that generates an immune response against one or more Dengue virus subtypes.

Specific Embodiment 12. The Dengue virus immunogen of Specific Embodiment 11, wherein the immunogen is Dengue virus E protein, Dengue virus prM/E protein, or an antibody-binding portion thereof.

Specific Embodiment 13. The Dengue virus immunogen of Specific Embodiment 11 or Embodiment 12, wherein the one or more Dengue virus subtypes is selected from the group consisting of: DENV1, DENV2, DENV3, DENV4, and any combination(s) thereof.

Specific Embodiment 14. The Dengue virus antigen, immunogen, VLP, or SVP of any one of Specific Embodiments 1-13, wherein the immune response comprises the production of neutralizing antibodies and/or T-lymphocytes.

Specific Embodiment 15. A pharmaceutically acceptable composition comprising the Dengue virus antigen, immunogen, VLP, or SVP of any one of Specific Embodiments 1-14 and a pharmaceutically acceptable carrier.

Specific Embodiment 16. The pharmaceutically acceptable composition of Specific Embodiment 15, further comprising an adjuvant.

Specific Embodiment 17. An immunogenic composition or vaccine comprising the Dengue virus antigen, immunogen, VLP, or SVP of any one of Specific Embodiments 1-14.

Specific Embodiment 18. A pharmaceutically acceptable composition comprising the immunogenic composition or vaccine of Specific Embodiment 17, and a pharmaceutically acceptable carrier, diluent, or excipient.

Specific Embodiment 19. The composition of Specific Embodiment 18, further comprising an adjuvant.

Specific Embodiment 20. A method of generating an immune response in a subject, the method comprising administering to the subject an effective amount of the Dengue virus antigen, immunogen, VLP, or SVP of any one of Specific Embodiments 1-14.

Specific Embodiment 21. A method of generating an immune response in a subject, the method comprising administering to the subject an effective amount of the pharmaceutical composition of any one of Specific Embodiments 15, 16, 18, and 19.

Specific Embodiment 22. A method of generating an immune response in a subject, the method comprising administering to the subject an effective amount of the immunogenic composition or vaccine of Specific Embodiment 17.

Specific Embodiment 23. The method of any one of Specific Embodiments 20-22, wherein the immune response comprises the production of neutralizing antibodies.

Specific Embodiment 24. The method of Specific Embodiment 23, wherein the immune response further comprises the production of T-lymphocytes.

Specific Embodiment 25. The method of any one of Specific Embodiments 20-24, wherein an adjuvant is concomitantly administered to the subject.

Specific Embodiment 26. A polynucleotide encoding the Dengue virus antigen of any one of Specific Embodiments 1-6.

Specific Embodiment 27. A composition comprising the polynucleotide of Specific Embodiment 26, and a pharmaceutically acceptable carrier, diluent, or excipient.

Specific Embodiment 28. Use of an effective amount of a pharmaceutically acceptable composition of Specific Embodiment 15 or Specific Embodiment 16, an immunogenic composition of Specific Embodiment 17, or a pharmaceutically acceptable composition of Specific Embodiment 18 or Specific Embodiment 19 comprising a non-naturally occurring, broadly reactive, pan-epitopic, Dengue virus antigen, antigen polypeptide, immunogen, VLP, or SVP containing a Dengue virus protein (e.g., DENV envelope (E) protein (e.g., SEQ ID NOs:1-7, etc.), DENV M protein, DENV prM protein, DENV prM/E protein (e.g., SEQ ID NOs:8-11, etc.), etc.), DENV protein SVPs, DENV protein VLPs, etc., fragments or combinations thereof, etc.) for the treatment, prevention, and/or attenuation of symptoms of Dengue virus, wherein the use produces an immune response.

Specific Embodiment 29. An effective amount of a Dengue virus a pharmaceutically acceptable composition of Specific Embodiment 15 or Specific Embodiment 16, an immunogenic composition of Specific Embodiment 17, or a pharmaceutically acceptable composition of Specific Embodiment 18 or Specific Embodiment 19 comprising a non-naturally occurring, broadly reactive, pan-epitopic, Dengue virus antigen or immunogen (e.g., DENV envelope (E) polypeptide (e.g., SEQ ID NOs:1-7, etc.), DENV M polypeptide, DENV prM polypeptide, DENV prM/E polypeptide (e.g., SEQ ID NOs:8-11, etc.), etc.), DENV polypeptide SVPs, DENV polypeptide VLPs, etc., fragments or combinations thereof) for use in the treatment, prevention, protection, or attenuation of Dengue virus and/or its symptoms or for the treatment, prevention, protection, or attenuation of Dengue virus and/or its symptoms.

Specific Embodiment 30. Use of a Dengue virus substance of Specific Embodiments 1-14, or a pharmaceutically acceptable composition of Specific Embodiment 15 or Specific Embodiment 16, an immunogenic composition of Specific Embodiment 17, or a pharmaceutically acceptable composition of Specific Embodiment 18 or Specific Embodiment 19 comprising a non-naturally occurring, broadly reactive, pan-epitopic, Dengue virus antigen or immunogen (e.g., DENV envelope (E) polypeptide (e.g., SEQ ID NOs:1-7, etc.), DENV M polypeptide, DENV prM polypeptide, DENV prM/E polypeptide (e.g., SEQ ID NOs:8-11, etc.), etc.), DENV polypeptide SVPs, DENV polypeptide VLPs, etc., fragments or combinations thereof) for the manufacture of a medicament for a therapeutic, prophylactic, or preventative application of Dengue virus and/or its symptoms.

Specific Embodiment 31. A method of preparing a Dengue virus substance of Specific Embodiments 1-14, or a pharmaceutically acceptable composition of Specific Embodiment 15 or Specific Embodiment 16, an immunogenic composition of Specific Embodiment 17, or a pharmaceutically acceptable composition of Specific Embodiment 18 or Specific Embodiment 19 comprising a non-naturally occurring, broadly reactive, pan-epitopic, Dengue virus antigen or immunogen (e.g., DENV envelope (E) polypeptide (e.g., SEQ ID NOs:1-7, etc.), DENV M polypeptide, DENV prM polypeptide, DENV prM/E polypeptide (e.g., SEQ ID NOs:8-11, etc.), etc.), DENV polypeptide SVPs, DENV polypeptide VLPs, etc., fragments or combinations thereof) for the manufacture of a medicament for a therapeutic, prophylactic, or preventative application of Dengue virus and/or its symptoms or for the treatment, prevention, protection, or attenuation of Dengue virus and/or its symptoms.

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1-27. (canceled)

28. A method of generating an immune response in a subject, the method comprising administering to the subject an effective amount of a Dengue virus (DENV) antigen, immunogen, VLP, or SVP, wherein the Dengue virus antigen, immunogen, VLP, or SVP comprises an amino acid sequence that is at least 90% identical to an amino acid sequence selected from SEQ ID NOs:8-11.

29. A method of generating an immune response in a subject, the method comprising administering to the subject an effective amount of a pharmaceutical composition comprising a Dengue virus (DENV) antigen, immunogen, VLP, or SVP, and a pharmaceutically acceptable carrier, wherein the Dengue virus antigen, immunogen, VLP, or SVP comprises an amino acid sequence that is at least 90% identical to an amino acid sequence selected from SEQ ID NOs:8-11.

30. A method of generating an immune response in a subject, the method comprising administering to the subject an effective amount of an immunogenic composition or vaccine comprising a Dengue virus (DENV) antigen, immunogen, VLP, or SVP, wherein the Dengue virus antigen, immunogen, VLP, or SVP comprises an amino acid sequence that is at least 90% identical to an amino acid sequence as set forth in SEQ ID NOs:8-11.

31. The method of claim 30, wherein the immune response comprises the production of neutralizing antibodies.

32. The method of claim 30, wherein the immune response further comprises the production of T-lymphocytes.

33. The method of claim 30, wherein an adjuvant is concomitantly administered to the subject.

34. A non-naturally occurring, pan-epitopic immunogen that generates an immune response against one or more Dengue virus subtypes.

35. The Dengue virus immunogen of claim 34, wherein the immunogen is Dengue virus E protein, Dengue virus prM/E protein, or an antibody-binding portion thereof.

36. The Dengue virus immunogen of claim 34, wherein the immunogen is a non-naturally occurring, broadly reactive, pan-epitopic antigen of Dengue virus.

37. The Dengue virus immunogen of claim 34, wherein the one or more Dengue virus subtypes is selected from the group consisting of: DENV1, DENV2, DENV3, DENV4, and any combination thereof.

38. The Dengue virus immunogen of claim 34, which comprises an amino acid sequence that is at least 90% identical to an amino acid sequence of a DENV antigen as set forth in FIGS. 9A, 9B, or SEQ ID NOs:8-11.

39. The Dengue virus immunogen of claim 34, which comprises an amino acid sequence that is at least 98% identical to an amino acid sequence of a DENV antigen as set forth in FIGS. 9A, 9B, or SEQ ID NOs:8-11.

40. A virus-like particle (VLP) comprising the Dengue virus antigen of claim 36.

41. The VLP of claim 40, which comprises a polynucleotide encoding the Dengue virus antigen.

42. A subviral particle (SVP) comprising the Dengue virus antigen of claim 36.

43. The SVP of claim 42 which comprises a polynucleotide encoding the Dengue virus antigen.

44. A pharmaceutically acceptable composition comprising the Dengue virus antigen, immunogen, VLP, or SVP of claim 34 or polynucleotide encoding the Dengue virus antigen, immunogen, VLP, or SVP, and a pharmaceutically acceptable carrier.

45. An immunogenic composition or vaccine comprising the Dengue virus antigen, immunogen, VLP, or SVP of claim 34 or polynucleotide encoding the Dengue virus antigen, immunogen, VLP, or SVP.

46. A pharmaceutically acceptable composition comprising the immunogenic composition or vaccine of claim 45 and a pharmaceutically acceptable carrier, diluent, or excipient.

47. A composition comprising the polynucleotide of claim 44, and a pharmaceutically acceptable carrier, diluent, or excipient.
